SEQUENCE LISTING (122 sequences)

SEQ ID NO: 1 - protein, 591 aa, Mycoplasma pneumoniae
MPNPVRFVYRVDLRSPEEIFEHGFSTLGDVRNFFEHILSTNFGRSYFISTSETPTAAIRFFGSWLREYVPEHPRRAYLYEIRADQHFYNARATGENLLDLMRQRQVVFDSGDREMAQMGIRALRTSFAYQREWFTDGPIAAANVRSAWLVDAVPVEPGHAHHPAGRVVETTRINEPEMHNPHYQELQTQANDQPWLPTPGIATPVHLSIPQAASVADVSEGTSASLSFACPDWSPPSSNGENPLDKCIAEKIDNYNLQSLPQYASSVKELEDTPVYLRGIKTQKTFMLQADPQNNNVFLVEVNPKQKSSFPQTIFFWDVYQRICLKDLTGAQISLSLTAFTTQYAGQLKVHLSVSAVNAVNQKWKMTPQDIAITQFRVSSELLGQTENGLFWNTKSGGSQHDLYVCPLKNPPSDLEELQIIVDECTTHAQFVTMRAASTFFVDVQLGWYWRGYYYTPQLSGWSYQMKTPDGQIFYDLKTSKIFFVQDNQNVFFLHNKLNKQTGYSWDWVEWLKHDMNEDKDENFKWYFSRDDLTIPSVEGLNFRHIRCYADNQQLKVIISGSRWGGWYSTYDKVESNVEDKILVKDGFDRF

SEQ ID NO: 2 - protein, 591 aa, Mycoplasma pneumoniae
MPNPVRFVYRVDLRSPEEIFEHGFSTLGDVRNFFEHIPSTNFGRSYFISTSETPTAAIRFFGSWLREYVPEHPRRAYLYEIRADQHFYNARATGENLLDLMRQRQVVFDSGDREMAQMGIRALRTSFAYQREWFTDGPIAAANVRSAWLVDAVPVEPGHAHHPAGRVVETTRINEPEMHNPHYQELQTQANDQPWLPTPGIATPVHLSIPQAASVADVSEGTSASLSFACPDWSPPSSNGENPLDKCIAEKIDNYNLQSLPQYASSVKELEDTPVYLRGIKTQKTFMLQADPQNNNVFLVEVNPKQKSPFPQTIFFWDVYQRICLKDLTGAQISLSLTAFTTQYAGQLKVHLSVSAVNAVNQKWKMTPQDSAITQFRVSSELLGQTENGLSWNTKSGGSQHDLYVCPLKNPPSDLEELQIIVDECTTHAQFVTMRAASTFFVDVQLGWYWRGYYYTPQLSGWSYQMKTPDGQIFYDLKTSKIFFVQDNQNVFFLHNKLNKQTGYSWDWVEWLKHDMNEDKDENFKWYFSRDDLTIPSVEGLNFRHIRCYADNQQLKVIISGSRWGGWYSTYDKVESNVEDKILVKDGFDRF

SEQ ID NO: 3 - protein, 591 aa, Mycoplasma pneumoniae
MPNPVRFVYRVDLRSPEEIFEHGFSTLGDVRNFFEHILSTNFGRSYFISTSETPTAAIRFFGSWLREYVPEHPRRAYLYEIRADQHFYNARATGENLLDLMRQRQVVFDSGDREMAQMGIRALRTSFAYQREWFTDGPIAAANVRSAWLVDAVPVEPGHAHHPAGRVVETTRINEPEMHNPHYQELQTQANDQPWLPTPGIATPVHLSIPQAASVADVSEGTSASLSFACPDWSPPSSNGENPLDKCIAEKIDNYNLQSLPQYASSVKELEDTPVYLRGIKTQKTFMLQADPQNNNVFLVEVNPKQKSSFPQTIFFWDVYQRICLKDLTGAQISLSLTAFTTQYAGQLKVHLSVSAVNAVNQKWKMTPQDSAITQFRVSSELLGQTENGLFWNTKSGGSQHDLYVCPLKNPPSDLEELQIIVDECTTHAQFVTMRAASTFFVDVQLGWYWRGYYYTPQLSGWSYQMKTPDGQIFYDLKTSKIFFVQDNQNVFFLHNKLNKQTGYSWDWVEWLKHDMNEDKDENFKWYFSRDDLTIPSVEGLNFRHIRCYADNQQLKVIISGSRWGGWYSTYDKVESNVEDKILVKDGFDRF

SEQ ID NO: 4 - protein, 591 aa, Mycoplasma pneumoniae
MPNPVRFVYRVDLRSPEEIFEHGFSTLGDVRNFFEHILSTNFGRSYFISTSETPTAAIRFFGSWLREYVPEHPRRAYLYEIRADQHFYNARATGENLLDLMRQRQVVFDSGDREMAQMGIRALRTSFAYQREWFTDGPIAAANVRSAWLVDAVPVEPGHAHHPAGRVVETTRINEPEMHNPHYQELQTQANDQPWLPTPGIATPVHLSIPQAASVADVSEGTSASLSFACPDWSPPSSNGENPLDKCIAEKIDNYNLQSLPQYASSVKELEDTPVYLRGIKTQKTFMLQADPQNNNVFLVEVNPKQKSSFPQTIFFWDVYQRICLKDLTGAQISLSLTAFTTQYAGQLKVHLSVSAVNAVNQKWKMTPQDSAITQFRVSSELLGQTENGLFRNTKSGGSQHDLYVCPLKNPPSDLEELQIIVDECTTHAQFVTMRAASTFFVDVQLGWYWRGYYYTPQLSGWSYQMKTPDGQIFYDLKTSKIFFVQDNQNVFFLHNKLNKQTGYSWDWVEWLKHDMNEDKDENFKWYFSRDDLTIPSVEGLNFRHIRCYADNQQLKVIISGSRWGGWYSTYDKVESNVEDKILVKDGFDRF

SEQ ID NO: 5 - protein, 591 aa, Mycoplasma pneumoniae
MPNPVRFVYRVDLRSPEEIFEHGFSTLGDVRNFFEHILSTNFGRSYFISTSETPTAAIRFFGSWLREYVPEHPRRAYLYEIRADQHFYNARATGENLLDLMRQRQVVFDSGDREMAQMGIRALRTSFAYQREWFTDGPIAAANVRSAWLVDAVPVEPGHAHHPAGRVVETTRINEPEMHNPHYQELQTQANDQPWLPTPGIATPVHLSIPQAASVADVSEGTSASLSFACPDWSPPSSNGENPLGKCIAEKIDNYNLQSLPQYASSVKELEDTPVYLRGIKTQKTFMLQADPQNNNVFLVEVNPKQKSSFPQTIFFWDVYQRICLKDLTGAQISLSLTAFTTQYAGQLKVHLSVSAVNAVNQKWKMTPQDSAITQFRVSSELLGQTENGLFWNTKSGGSQHDLYVCPLKNPPSDLEELQIIVDECTTHAQFVTMRAASTFFVDVQLGWYWRGYYYTPQLSGWSYQMKTPDGQIFYDLKTSKIFFVQDNQNVFFLHNKLNKQTGYSWDWVEWLKHDMNEDKDENFKWYFSRDDLTIPSVEGLNFRHIRCYADNQQLKVIISGSRWGGWYSTYDKVESNVEDKILVKDGFDRF

SEQ ID NO: 6 - protein, 591 aa, Artificial
  Composite amino acid sequence
MPNPVRFVYRVDLRSPEEIFEHGFSTLGDVRNFFEHIPSTNFGRSYFISTSETPTAAIRFFGSWLREYVPEHPRRAYLYEIRADQHFYNARATGENLLDLMRQRQVVFDSGDREMAQMGIRALRTSFAYQREWFTDGPIAAANVRSAWLVDAVPVEPGHAHHPAGRVVETTRINEPEMHNPHYQELQTQANDQPWLPTPGIATPVHLSIPQAASVADVSEGTSASLSFACPDWSPPSSNGENPLGKCIAEKIDNYNLQSLPQYASSVKELEDTPVYLRGIKTQKTFMLQADPQNNNVFLVEVNPKQKPSFPQTIFFWDVYQRICLKDLTGAQISLSLTAFTTQYAGQLKVHLSVSAVNAVNQKWKMTPQDSAITQFRVSSELLGQTENGLSRNTKSGGSQHDLYVCPLKNPPSDLEELQIIVDECTTHAQFVTMRAASTFFVDVQLGWYWRGYYYTPQLSGWSYQMKTPDGQIFYDLKTSKIFFVQDNQNVFFLHNKLNKQTGYSWDWVEWLKHDMNEDKDENFKWYFSRDDLTIPSVEGLNFRHIRCYADNQQLKVIISGSRWGGWYSTYDKVESNVEDKILVKDGFDRF

SEQ ID NO: 7 - DNA, 1860 bp, Mycoplasma pneumoniae
tttttaatttgtaaaatttcattttttaaaaatgccaaatcctgttagatttgtttaccgtgttgatttgagaagccctgaagaaatttttgaacatggcttttcaactttaggtgatgtgagaaatttctttgaacacattctctccactaattttggtagaagctattttatttccacttcagaaacacccacagcagctattcgcttctttggtagctggttacgggaatatgtaccagagcaccccagaagggcttacttatatgaaattcgtgccgaccaacacttttacaatgcccgcgccactggggagaacttgttagatttaatgcgtcaaagacaagtagtatttgactctggtgatcgagaaatggcacaaatgggaattagagctttacgcacttcctttgcgtatcaacgtgaatggtttaccgatggtccaattgcagcagctaatgtccgtagtgcttgactagtagatgctgttcccgttgaacctggtcatgctcaccacccggctggtcgtgttgtagagactactagaattaatgaaccggaaatgcacaaccctcattatcaagagctgcaaacccaagccaatgatcaaccatgattgccaacaccaggaatagctactcctgtacatttatcaattccccaagcagcttccgttgctgatgtttcggaaggtacttccgcttcgctatcgtttgcgtgccctgattgaagtccaccttctagtaatggtgaaaatccgctagacaaatgcattgcggaaaagattgataactataacctacaatccttaccacagtacgctagcagtgtaaaggaactggaagatacaccagtatacctaaggggaattaaaacgcaaaaaacctttatgttacaagcagatccgcaaaataacaatgtctttttggtcgaagtaaaccccaaacaaaagtccagctttccccaaaccatcttcttttgggatgtttatcaacgaatttgtctcaaggatttaactggtgcacaaatcagtctttcgcttactgcctttactactcagtatgctggtcagctcaaagtgcaccttagtgttagcgcggttaatgccgtgaaccaaaagtgaaaaatgacaccgcaagacattgcaataactcagtttcgggtctcctctgaactgttaggtcaaactgaaaatggcttgttctgaaataccaagagtggtggttcacaacacgatttgtatgtatgtcctttgaaaaatccacctagtgatttggaagaattacaaataattgttgatgaatgtactacccatgcgcagtttgttactatgcgtgcagctagcaccttctttgttgatgttcagctaggctggtattgaaggggttattactataccccacaattaagtggttgatcttatcagatgaaaacaccagatggacagatattctatgatctaaaaacttcgaaaatcttctttgtccaggacaaccaaaacgtgttctttctccataataaactcaacaaacaaactggttacagctgggattgagtagaatggctaaaacatgacatgaatgaggacaaagacgaaaactttaaatggtacttttcgcgtgatgaccttaccattccttccgttgaagggcttaacttccgccacattcgctgttacgctgacaaccagcagttaaaggtgatcataagcggttcacgttggggcggttggtactccacttacgataaagttgaaagtaatgtcgaagataagattttggtcaaagatggttttgatcgcttttagcgattaagctttaacgtcactgttttgctctaatgttagaagcaaagatcttg

SEQ ID NO: 8 - DNA, 1773 bp, Artificial
  S1 nucleotide sequence with tga codons changed to tgg for expression in E. coli
atgccaaatcctgttagatttgtttaccgtgttgatttgagaagccctgaagaaatttttgaacatggcttttcaactttaggtgatgtgagaaatttctttgaacacattccctccactaattttggtagaagctattttatttccacttcagaaacacccacagcagctattcgcttctttggtagctggttacgggaatatgtaccagagcaccccagaagggcttacttatatgaaattcgtgccgaccaacacttttacaatgcccgcgccactggggagaacttgttagatttaatgcgtcaaagacaagtagtatttgactctggtgatcgagaaatggcacaaatgggaattagagctttacgcacttcctttgcgtatcaacgtgaatggtttaccgatggtccaattgcagcagctaatgtccgtagtgcttggctagtagatgctgttcccgttgaacctggtcatgctcaccacccggctggtcgtgttgtagagactactagaattaatgaaccggaaatgcacaaccctcattatcaagagctgcaaacccaagccaatgatcaaccatggttgccaacaccaggaatagctactcctgtacatttatcaattccccaagcagcttccgttgctgatgtttcggaaggtacttccgcttcgctatcgtttgcgtgccctgattggagtccaccttctagtaatggtgaaaatccgctagacaaatgcattgcggaaaagattgataactataacctacaatccttaccacagtacgctagcagtgtaaaggaactggaagatacaccagtatacctaaggggaattaaaacgcaaaaaacctttatgttacaagcagatccgcaaaataacaatgtctttttggtcgaagtaaaccccaaacaaaagcccagctttccccaaaccatcttcttttgggatgtttatcaacgaatttgtctcaaggatttaactggtgcacaaatcagtctttcgcttactgcctttactactcagtatgctggtcagctcaaagtgcaccttagtgttagcgcggttaatgccgtgaaccaaaagtggaaaatgacaccgcaagacagtgcaataactcagtttcgggtctcctctgaactgttaggtcaaactgaaaatggcttgtcctggaataccaagagtggtggttcacaacacgatttgtatgtatgtcctttgaaaaatccacctagtgatttggaagaattacaaataattgttgatgaatgtactacccatgcgcagtttgttactatgcgtgcagctagcaccttctttgttgatgttcagctaggctggtattggaggggttattactataccccacaattaagtggttggtcttatcagatgaaaacaccagatggacagatattctatgatctaaaaacttcgaaaatcttctttgtccaggacaaccaaaacgtgttctttctccataataaactcaacaaacaaactggttacagctgggattgggtagaatggctaaaacatgacatgaatgaggacaaagacgaaaactttaaatggtacttttcgcgtgatgaccttaccattccttccgttgaagggcttaacttccgccacattcgctgttacgctgacaaccagcagttaaaggtgatcataagcggttcacgttggggcggttggtactccacttacgataaagttgaaagtaatgtcgaagataagattttggtcaaagatggttttgatcgcttt

SEQ ID NO: 9 - DNA, 1773 bp, Artificial
  L2 nucleotide sequence with tga codons changed to tgg for expression in E. Coli
atgccaaatcctgttagatttgtttaccgtgttgatttgagaagccctgaagaaatttttgaacatggcttttcaactttaggtgatgtgagaaatttctttgaacacattctctccactaattttggtagaagctattttatttccacttcagaaacacccacagcagctattcgcttctttggtagctggttacgggaatatgtaccagagcaccccagaagggcttacttatatgaaattcgtgccgaccaacacttttacaatgcccgcgccactggggagaacttgttagatttaatgcgtcaaagacaagtagtatttgactctggtgatcgagaaatggcacaaatgggaattagagctttacgcacttcctttgcgtatcaacgtgaatggtttaccgatggtccaattgcagcagctaatgtccgtagtgcttggctagtagatgctgttcccgttgaacctggtcatgctcaccacccggctggtcgtgttgtagagactactagaattaatgaaccggaaatgcacaaccctcattatcaagagctgcaaacccaagccaatgatcaaccatggttgccaacaccaggaatagctactcctgtacatttatcaattccccaagcagcttccgttgctgatgtttcggaaggtacttccgcttcgctatcgtttgcgtgccctgattggagtccaccttctagtaatggtgaaaatccgctaggcaaatgcattgcggaaaagattgataactataacctacaatccttaccacagtacgctagcagtgtaaaggaactggaagatacaccagtatacctaaggggaattaaaacgcaaaaaacctttatgttacaagcagatccgcaaaataacaatgtctttttggtcgaagtaaaccccaaacaaaagtccagctttccccaaaccatcttcttttgggatgtttatcaacgaatttgtctcaaggatttaactggtgcacaaatcagtctttcgcttactgcctttactactcagtatgctggtcagctcaaagtgcaccttagtgttagcgcggttaatgccgtgaaccaaaagtggaaaatgacaccgcaagacagtgcaataactcagtttcgggtctcctctgaactgttaggtcaaactgaaaatggcttgttctggaataccaagagtggtggttcacaacacgatttgtatgtatgtcctttgaaaaatccacctagtgatttggaagaattacaaataattgttgatgaatgtactacccatgcgcagtttgttactatgcgtgcagctagcaccttctttgttgatgttcagctaggctggtattggaggggttattactataccccacaattaagtggttggtcttatcagatgaaaacaccagatggacagatattctatgatctaaaaacttcgaaaatcttctttgtccaggacaaccaaaacgtgttctttctccataataaactcaacaaacaaactggttacagctgggattgggtagaatggctaaaacatgacatgaatgaggacaaagacgaaaactttaaatggtacttttcgcgtgatgaccttaccattccttccgttgaagggcttaacttccgccacattcgctgttacgctgacaaccagcagttaaaggtgatcataagcggttcacgttggggcggttggtactccacttacgataaagttgaaagtaatgtcgaagataagattttggtcaaagatggttttgatcgcttt

SEQ ID NO: 10 - DNA, 1773 bp, Artificial
  JL nucleotide sequence with tga codons changed to tgg for expression in E. coli
atgccaaatcctgttagatttgtttaccgtgttgatttgagaagccctgaagaaatttttgaacatggcttttcaactttaggtgatgtgagaaatttctttgaacacattctctccactaattttggtagaagctattttatttccacttcagaaacacccacagcagctattcgcttctttggtagctggttacgggaatatgtaccagagcaccccagaagggcttacttatatgaaattcgtgccgaccaacacttttacaatgcccgcgccactggggagaacttgttagatttaatgcgtcaaagacaagtagtatttgactctggtgatcgagaaatggcacaaatgggaattagagctttacgcacttcctttgcgtatcaacgtgaatggtttaccgatggtccaattgcagcagctaatgtccgtagtgcttggctagtagatgctgttcccgttgaacctggtcatgctcaccacccggctggtcgtgttgtagagactactagaattaatgaaccggaaatgcacaaccctcattatcaagagctgcaaacccaagccaatgatcaaccatggttgccaacaccaggaatagctactcctgtacatttatcaattccccaagcagcttccgttgctgatgtttcggaaggtacttccgcttcgctatcgtttgcgtgccctgattggagtccaccttctagtaatggtgaaaatccgctagacaaatgcattgcggaaaagattgataactataacctacaatccttaccacagtacgctagcagtgtaaaggaactggaagatacaccagtatacctaaggggaattaaaacgcaaaaaacctttatgttacaagcagatccgcaaaataacaatgtctttttggtcgaagtaaaccccaaacaaaagtccagctttccccaaaccatcttcttttgggatgtttatcaacgaatttgtctcaaggatttaactggtgcacaaatcagtctttcgcttactgcctttactactcagtatgctggtcagctcaaagtgcaccttagtgttagcgcggttaatgccgtgaaccaaaagtggaaaatgacaccgcaagacagtgcaataactcagtttcgggtctcctctgaactgttaggtcaaactgaaaatggcttgttctggaataccaagagtggtggttcacaacacgatttgtatgtatgtcctttgaaaaatccacctagtgatttggaagaattacaaataattgttgatgaatgtactacccatgcgcagtttgttactatgcgtgcagctagcaccttctttgttgatgttcagctaggctggtattggaggggttattactataccccacaattaagtggttggtcttatcagatgaaaacaccagatggacagatattctatgatctaaaaacttcgaaaatcttctttgtccaggacaaccaaaacgtgttctttctccataataaactcaacaaacaaactggttacagctgggattgggtagaatggctaaaacatgacatgaatgaggacaaagacgaaaactttaaatggtacttttcgcgtgatgaccttaccattccttccgttgaagggcttaacttccgccacattcgctgttacgctgacaaccagcagttaaaggtgatcataagcggttcacgttggggcggttggtactccacttacgataaagttgaaagtaatgtcgaagataagattttggtcaaagatggttttgatcgcttt

SEQ ID NO: 11 - DNA, 1773 bp, Artificial
  RJL1 nucleotide sequence with tga codons changed to tgg for expression in E. coli
atgccaaatcctgttagatttgtttaccgtgttgatttgagaagccctgaagaaatttttgaacatggcttttcaactttaggtgatgtgagaaatttctttgaacacattctctccactaattttggtagaagctattttatttccacttcagaaacacccacagcagctattcgcttctttggtagctggttacgggaatatgtaccagagcaccccagaagggcttacttatatgaaattcgtgccgaccaacacttttacaatgcccgcgccactggggagaacttgttagatttaatgcgtcaaagacaagtagtatttgactctggtgatcgagaaatggcacaaatgggaattagagctttacgcacttcctttgcgtatcaacgtgaatggtttaccgatggtccaattgcagcagctaatgtccgtagtgcttggctagtagatgctgttcccgttgaacctggtcatgctcaccacccggctggtcgtgttgtagagactactagaattaatgaaccggaaatgcacaaccctcattatcaagagctgcaaacccaagccaatgatcaaccatggttgccaacaccaggaatagctactcctgtacatttatcaattccccaagcagcttccgttgctgatgtttcggaaggtacttccgcttcgctatcgtttgcgtgccctgattggagtccaccttctagtaatggtgaaaatccgctagacaaatgcattgcggaaaagattgataactataacctacaatccttaccacagtacgctagcagtgtaaaggaactggaagatacaccagtatacctaaggggaattaaaacgcaaaaaacctttatgttacaagcagatccgcaaaataacaatgtctttttggtcgaagtaaaccccaaacaaaagtccagctttccccaaaccatcttcttttgggatgtttatcaacgaatttgtctcaaggatttaactggtgcacaaatcagtctttcgcttactgcctttactactcagtatgctggtcagctcaaagtgcaccttagtgttagcgcggttaatgccgtgaaccaaaagtggaaaatgacaccgcaagacagtgcaataactcagtttcgggtctcctctgaactgttaggtcaaactgaaaatggcttgttccggaataccaagagtggtggttcacaacacgatttgtatgtatgtcctttgaaaaatccacctagtgatttggaagaattacaaataattgttgatgaatgtactacccatgcgcagtttgttactatgcgtgcagctagcaccttctttgttgatgttcagctaggctggtattggaggggttattactataccccacaattaagtggttggtcttatcagatgaaaacaccagatggacagatattctatgatctaaaaacttcgaaaatcttctttgtccaggacaaccaaaacgtgttctttctccataataaactcaacaaacaaactggttacagctgggattgggtagaatggctaaaacatgacatgaatgaggacaaagacgaaaactttaaatggtacttttcgcgtgatgaccttaccattccttccgttgaagggcttaacttccgccacattcgctgttacgctgacaaccagcagttaaaggtgatcataagcggttcacgttggggcggttggtactccacttacgataaagttgaaagtaatgtcgaagataagattttggtcaaagatggttttgatcgcttt

SEQ ID NO: 12 - DNA, 24 bp, Artificial
  Oligonucleotide primer
tttttacatatgccaaatcctgtt

SEQ ID NO: 13 - DNA, 28 bp, Artificial
  Oligonucleotide primer
cgttaaaggatcctcgctaaaagcgatc

SEQ ID NO: 14 - DNA, 25 bp, Artificial
  Oligonucleotide primer
ctagccaagcactacggacattagc

SEQ ID NO: 15 - DNA, 27 bp, Artificial
  Oligonucleotide primer
cgtagtgcttggctagtagatgctgtt

SEQ ID NO: 16 - DNA, 23 bp, Artificial
  Oligonucleotide primer
cctggtgttggcaaccatggttg

SEQ ID NO: 17 - DNA, 23 bp, Artificial
  Oligonucleotide primer
gatcaaccatggttgccaacacc

SEQ ID NO: 18 - DNA, 24 bp, Artificial
  Oligonucleotide primer
aaggtggactccaatcagggcacg

SEQ ID NO: 19 - DNA, 24 bp, Artificial
  Oligonucleotide primer
cgtgccctgattggagtccacctt

SEQ ID NO: 20 - DNA, 23 bp, Artificial
  Oligonucleotide primer
gcggtgtcattttccacttttgg

SEQ ID NO: 21 - DNA, 23 bp, Artificial
  Oligonucleotide primer
ccaaaagtggaaaatgacaccgc

SEQ ID NO: 22 - DNA, 22 bp, Artificial
  Oligonucleotide primer
ggtattccagaacaagccattt

SEQ ID NO: 23 - DNA, 24 bp, Artificial
  Oligonucleotide primer
gcttgttctggaataccaagagtg

SEQ ID NO: 24 - DNA, 21 bp, Artificial
  Oligonucleotide primer
ataacccctataccagcctag

SEQ ID NO: 25 - DNA, 59 bp, Artificial
  Oligonucleotide primer
gctggtattggaggggttattactataccccacaattaagtggttggtcttatcagatg

SEQ ID NO: 26 - DNA, 24 bp, Artificial
  Oligonucleotide primer
ccattctacccaatcccagctgta

SEQ ID NO: 27 - DNA, 24 bp, Artificial
  Oligonucleotide primer
tacagctgggattgggtagaatgg

SEQ ID NO: 28 - DNA, 24 bp, Mycoplasma pneumoniae
tttttaaaaatgccaaatcctgtt

SEQ ID NO: 29 - DNA, 20 bp, Mycoplasma pneumoniae
aatgtccgtagtgcttgact

SEQ ID NO: 30 - DNA, 20 bp, Artificial
  Modified M129/B9 sequence
aatgtccgtagtgcttggct

SEQ ID NO: 31 - DNA, 20 bp, Artificial
  Oligonucleotide primer
agccaagcactacggacatt

SEQ ID NO: 32 - DNA, 22 bp, Mycoplasma pneumoniae
tgcttgactagtagatgctgtt

SEQ ID NO: 33 - DNA, 22 bp, Artificial
  Oligonucleotide primer
tgcttggctagtagatgctgtt

SEQ ID NO: 34 - DNA, 18 bp, Mycoplasma pneumoniae
atgattgccaacaccagg

SEQ ID NO: 35 - DNA, 18 bp, Artificial
  Modified M129/B9 sequence
atggttgccaacaccagg

SEQ ID NO: 36 - DNA, 18 bp, Artificial
  Oligonucleotide primer
cctggtgttggcaaccat

SEQ ID NO: 37 - DNA, 18 bp, Mycoplasma pneumoniae
accatgattgccaacacc

SEQ ID NO: 38 - DNA, 18 bp, Artificial
  Oligonucleotide primer
accatggttgccaacacc

SEQ ID NO: 39 - DNA, 19 bp, Mycoplasma pneumoniae
cctgattgaagtccacctt

SEQ ID NO: 40 - DNA, 19 bp, Artificial
  Modified M129/B9 sequence
cctgattggagtccacctt

SEQ ID NO: 41 - DNA, 19 bp, Artificial
  Oligonucleotide primer
aaggtggactccaatcagg

SEQ ID NO: 42 - DNA, 18 bp, Mycoplasma pneumoniae
cgtgccctgattgaagtc

SEQ ID NO: 43 - DNA, 18 bp, Artificial
  Oligonucleotide primer
cgtgccctgattggagtc

SEQ ID NO: 44 - DNA, 20 bp, Mycoplasma pneumoniae
aaagtgaaaaatgacaccgc

SEQ ID NO: 45 - DNA, 20 bp, Artificial
  Modified M129/B9 sequence
aaagtggaaaatgacaccgc

SEQ ID NO: 46 - DNA, 20 bp, Artificial
  Oligonucleotide primer
gcggtgtcattttccacttt

SEQ ID NO: 47 - DNA, 20 bp, Mycoplasma pneumoniae
caaaagtgaaaaatgacacc

SEQ ID NO: 48 - DNA, 20 bp, Artificial
  Oligonucleotide primer
caaaagtggaaaatgacacc

SEQ ID NO: 49 - DNA, 22 bp, Mycoplasma pneumoniae
aaatggcttgttctgaaatacc

SEQ ID NO: 50 - DNA, 22 bp, Artificial
  Modified M129/B9 sequence
aaatggcttgttctggaatacc

SEQ ID NO: 51 - DNA, 23 bp, Mycoplasma pneumoniae
gcttgttctgaaataccaagagt

SEQ ID NO: 52 - DNA, 23 bp, Artificial
  Oligonucleotide primer
gcttgttctggaataccaagagt

SEQ ID NO: 53 - DNA, 20 bp, Mycoplasma pneumoniae
taggctggtattgaaggggt

SEQ ID NO: 54 - DNA, 20 bp, Artificial
  Modified M129/B9 sequence
taggctggtattggaggggt

SEQ ID NO: 55 - DNA, 17 bp, Artificial
  Oligonucleotide primer
acccctataccagccta

SEQ ID NO: 56 - DNA, 56 bp, Mycoplasma pneumoniae
ggtattgaaggggttattactataccccacaattaagtggttgatcttatcagatg

SEQ ID NO: 57 - DNA, 56 bp, Artificial
  Oligonucleotide primer
ggtattggaggggttattactataccccacaattaagtggttggtcttatcagatg

SEQ ID NO: 58 - DNA, 21 bp, Mycoplasma pneumoniae
tacagctgggattgagtagaa

SEQ ID NO: 59 - DNA, 21 bp, Artificial
  Modified M129/B9 sequence
tacagctgggattgggtagaa

SEQ ID NO: 60 - DNA, 21 bp, Artificial
  Oligonucleotide primer
ttctacccaatcccagctgta

SEQ ID NO: 61 - DNA, 21 bp, Mycoplasma pneumoniae
tacagctgggattgagtagaa

SEQ ID NO: 62 - DNA, 21 bp, Artificial
  Oligonucleotide primer
tacagctgggattgggtagaa

SEQ ID NO: 63 - DNA, 28 bp, Mycoplasma pneumoniae
gatcgcttttagcgattaagctttaacg

SEQ ID NO: 64 - DNA, 28 bp, Artificial
  Modified M129/B9 sequence
gatcgcttttagcgaggatcctttaacg

SEQ ID NO: 65 - DNA, 28 bp, Artificial
  Oligonucleotide primer
ggatcctctacgcaatgcatttgtctag

SEQ ID NO: 66 - DNA, 28 bp, Artificial
  Oligonucleotide primer
catatgccaacaccaggaatagctactc

SEQ ID NO: 67 - DNA, 25 bp, Artificial
  Oligonucleotide primer
ggatccactaccagcctagctgaac

SEQ ID NO: 68 - DNA, 29 bp, Artificial
  Oligonucleotide primer
catatgggtcagctcaaagtgcaccttag

SEQ ID NO: 69 - protein, 249 aa, Mycoplasma pneumoniae
MPNPVRFVYRVDLRSPEEIFEHGFSTLGDVRNFFEHILSTNFGRSYFISTSETPTAAIRFFGSWLREYVPEHPRRAYLYEIRADQHFYNARATGENLLDLMRQRQVVFDSGDREMAQMGIRALRTSFAYQREWFTDGPIAAANVRSAWLVDAVPVEPGHAHHPAGRVVETTRINEPEMHNPHYQELQTQANDQPWLPTPGIATPVHLSIPQAASVADVSEGTSASLSFACPDWSPPSSNGENPLDKCIA

SEQ ID NO: 70 - protein, 256 aa, Mycoplasma pneumoniae
PWLPTPGIATPVHLSIPQAASVADVSEGTSASLSFACPDWSPPSSNGENPLDKCIAEKIDNYNLQSLPQYASSVKELEDTPVYLRGIKTQKTFMLQADPQNNNVFLVEVNPKQKSSFPQTIFFWDVYQRICLKDLTGAQISLSLTAFTTQYAGQLKVHLSVSAVNAVNQKWKMTPQDIAITQFRVSSELLGQTENGLFWNTKSGGSQHDLYVCPLKNPPSDLEELQIIVDECTTHAQFVTMRAASTFFVDVQLGWY

SEQ ID NO: 71 - protein, 247 aa, Mycoplasma pneumoniae
AGQLKVHLSVSAVNAVNQKWKMTPQDIAITQFRVSSELLGQTENGLFWNTKSGGSQHDLYVCPLKNPPSDLEELQIIVDECTTHAQFVTMRAASTFFVDVQLGWYWRGYYYTPQLSGWSYQMKTPDGQIFYDLKTSKIFFVQDNQNVFFLHNKLNKQTGYSWDWVEWLKHDMNEDKDENFKWYFSRDDLTIPSVEGLNFRHIRCYADNQQLKVIISGSRWGGWYSTYDKVESNVEDKILVKDGFDRF

SEQ ID NO: 72 - DNA, 26 bp, Artificial
  Oligonucleotide primer
tttttacatatgccaaatcctgttag

SEQ ID NO: 73 - DNA, 24 bp, Artificial
  Oligonucleotide primer
ataacccctccaataccagcctag

SEQ ID NO: 74 - DNA, 747 bp, Artificial
  Amplified region of MNP372-D1
atgccaaatcctgttagatttgtttaccgtgttgatttgagaagccctgaagaaatttttgaacatggcttttcaactttaggtgatgtgagaaatttctttgaacacattctctccactaattttggtagaagctattttatttccacttcagaaacacccacagcagctattcgcttctttggtagctggttacgggaatatgtaccagagcaccccagaagggcttacttatatgaaattcgtgccgaccaacacttttacaatgcccgcgccactggggagaacttgttagatttaatgcgtcaaagacaagtagtatttgactctggtgatcgagaaatggcacaaatgggaattagagctttacgcacttcctttgcgtatcaacgtgaatggtttaccgatggtccaattgcagcagctaatgtccgtagtgcttggctagtagatgctgttcccgttgaacctggtcatgctcaccacccggctggtcgtgttgtagagactactagaattaatgaaccggaaatgcacaaccctcattatcaagagctgcaaacccaagccaatgatcaaccatggttgccaacaccaggaatagctactcctgtacatttatcaattccccaagcagcttccgttgctgatgtttcggaaggtacttccgcttcgctatcgtttgcgtgccctgattggagtccaccttctagtaatggtgaaaatccgctagacaaatgcattgcg

SEQ ID NO: 75 - protein, 272 aa, Artificial
  MPN372-D1 with His tag
MGHHHHHHHHHHSSGHIDDDDKHMPNPVRFVYRVDLRSPEEIFEHGFSTLGDVRNFFEHILSTNFGRSYFISTSETPTAAIRFFGSWLREYVPEHPRRAYLYEIRADQHFYNARATGENLLDLMRQRQVVFDSGDREMAQMGIRALRTSFAYQREWFTDGPIAAANVRSAWLVDAVPVEPGHAHHPAGRVVETTRINEPEMHNPHYQELQTQANDQPWLPTPGIATPVHLSIPQAASVADVSEGTSASLSFACPDWSPPSSNGENPLDKCIA

SEQ ID NO: 76 - DNA, 1773 bp, Artificial
  Degenerate DNA coding sequence to SEQ ID NO6
atgccnaayccngtnmgnttygtntaymgngtngayytnmgnwsnccngargarathttygarcayggnttywsnacnytnggngaygtnmgnaayttyttygarcayathccnwsnacnaayttyggnmgnwsntayttyathwsnacnwsngaracnccnacngcngcnathmgnttyttyggnwsntggytnmgngartaygtnccngarcayccnmgnmgngcntayytntaygarathmgngcngaycarcayttytayaaygcnmgngcnacnggngaraayytnytngayytnatgmgncarmgncargtngtnttygaywsnggngaymgngaratggcncaratgggnathmgngcnytnmgnacnwsnttygcntaycarmgngartggttyacngayggnccnathgcngcngcnaaygtnmgnwsngcntggytngtngaygcngtnccngtngarccnggncaygcncaycayccngcnggnmgngtngtngaracnacnmgnathaaygarccngaratgcayaayccncaytaycargarytncaracncargcnaaygaycarccntggytnccnacnccnggnathgcnacnccngtncayytnwsnathccncargcngcnwsngtngcngaygtnwsngarggnacnwsngcnwsnytnwsnttygcntgyccngaytggwsnccnccnwsnwsnaayggngaraayccnytnggnaartgyathgcngaraarathgayaaytayaayytncarwsnytnccncartaygcnwsnwsngtnaargarytngargayacnccngtntayytnmgnggnathaaracncaraaracnttyatgytncargcngayccncaraayaayaaygtnttyytngtngargtnaayccnaarcaraarccnwsnttyccncaracnathttyttytgggaygtntaycarmgnathtgyytnaargayytnacnggngcncarathwsnytnwsnytnacngcnttyacnacncartaygcnggncarytnaargtncayytnwsngtnwsngcngtnaaygcngtnaaycaraartggaaratgacnccncargaywsngcnathacncarttymgngtnwsnwsngarytnytnggncaracngaraayggnytnwsnmgnaayacnaarwsnggnggnwsncarcaygayytntaygtntgyccnytnaaraayccnccnwsngayytngargarytncarathathgtngaygartgyacnacncaygcncarttygtnacnatgmgngcngcnwsnacnttyttygtngaygtncarytnggntggtaytggmgnggntaytaytayacnccncarytnwsnggntggwsntaycaratgaaracnccngayggncarathttytaygayytnaaracnwsnaarathttyttygtncargayaaycaraaygtnttyttyytncayaayaarytnaayaarcaracnggntaywsntgggaytgggtngartggytnaarcaygayatgaaygargayaargaygaraayttyaartggtayttywsnmgngaygayytnacnathccnwsngtngarggnytnaayttymgncayathmgntgytaygcngayaaycarcarytnaargtnathathwsnggnwsnmgntggggnggntggtaywsnacntaygayaargtngarwsnaaygtngargayaarathytngtnaargayggnttygaymgntty

SEQ ID NO: 77 - DNA, 1944 bp, Mycoplasma penetrans
atgattaacaatgatattaaatcatcttgattacctgaatattattcattattagaaaataaaccaccaaaatttttatatagagttgattacaaaagccctgaagaagtttttcaaaaaggattttcaattaatagtcaaaatgagtttagtttttttgactatttttttaatatttattcttgtactagtactgaaaaagaagattgtttcattaatgcttttgaaacagtaaatgaagcaataattaattttagaaaaaatttatatgcaatcaatggaaatataaaagatctatatttgtatttgattagatgtgatgaaaattttttcagtaaacatataactagatgtacatatccttctgcacttattgaaaaccaagttatatcaaataacaaaaatgaaacaaataagttaatttttgcatttagtgattatggtcaaaaatttactaataaatttgaatggttcacaactaaaaaaattagtagtaaccaagtgttttctgcttcgcatattaaattaaattttaaaaagaattcaaatactaaaaataaaaatgactttacaataatcccggaaattgaagatactatctttagaaatcctaattatttagatttgaatactcaagcaaatttaagggcttttatatatcctgaatatttagcttcaaaaaaaattgaatttaaaaatgaagcttattattttgaacacaatgacaaaatatcttcatgaatgaatgttcaaaataaatcatttgatagtaacaatattattaaacacagaaaagataaaccaattgttaaacaaattactttgtttgataaaaacaataaaaaaaagattattaaagttaacttttacaaagaagaattaagttcacttttttatgatgtgtttatggaagaacaaagatgtttaaactatggttataaacaaccaaaaccctttgaactattatttacttatgaaaaattccaagataaaagtgtttacttaaatgcaagtactactaaaaaaaggggaagaatattttttgtttcaaaaacaaaaaataaagaagatattaataaaatttattttgataaatcaggtagatttatttttgacttcaataaagaatctgtgccttttgcaatcacactaacaaattatgataaatctaaagatattgctgaatttgatatgttgccagcttgtgagaataaccctaatcaaaattttcacttggagcacgctcattcaagtcacttctatttaaaaccaagtaataaaatattccagcatttggaattagcaattaagcataataataattcttttgttttcttaaaccctaaaaagaaatatagttttgcatatgatttaattaaccttaacttatctaagtattctaaaaatacacaaaactttatttatggaatgaatgtacatgttccagaattaattaacattaatttatcatgaatgtgaaaatatcaatattttaaacctaatatttttcttacatataatgacgaaaaaaatgatgtcaaaatagaacaagcaagatcaaatgaaaacaaaagtgatttatctaacttattatattgtttaaatactttaagtattttaatggttgattacaataaccactataaacttaatactgatatttttgcaatgaagaataatgcaaaagatgggaaaccatatagatggttagaatgacagaaagttaatttatcaaaatatccaaataaaaataatatgtgagttttgaaaaaatgtagatatgacaaagataatttttattggattatcagttttttcaataatgattatttatgggtgcaacaaaaaggtgaaaattgaggttttttctttttagcaaataaaaaaagtaagcctttaaaatcatcttcaatattttttcttaatgaaaatcatattaagtaa

SEQ ID NO: 78 - DNA, 1944 bp, Artificial
  M. penetrans ADP ribosylating and vacuolating toxin nucleotide sequence modified for expression in E. coli
atgattaacaatgatattaaatcatcttggttacctgaatattattcattattagaaaataaaccaccaaaatttttatatagagttgattacaaaagccctgaagaagtttttcaaaaaggattttcaattaatagtcaaaatgagtttagtttttttgactatttttttaatatttattcttgtactagtactgaaaaagaagattgtttcattaatgcttttgaaacagtaaatgaagcaataattaattttagaaaaaatttatatgcaatcaatggaaatataaaagatctatatttgtatttgattagatgtgatgaaaattttttcagtaaacatataactagatgtacatatccttctgcacttattgaaaaccaagttatatcaaataacaaaaatgaaacaaataagttaatttttgcatttagtgattatggtcaaaaatttactaataaatttgaatggttcacaactaaaaaaattagtagtaaccaagtgttttctgcttcgcatattaaattaaattttaaaaagaattcaaatactaaaaataaaaatgactttacaataatcccggaaattgaagatactatctttagaaatcctaattatttagatttgaatactcaagcaaatttaagggcttttatatatcctgaatatttagcttcaaaaaaaattgaatttaaaaatgaagcttattattttgaacacaatgacaaaatatcttcatggatgaatgttcaaaataaatcatttgatagtaacaatattattaaacacagaaaagataaaccaattgttaaacaaattactttgtttgataaaaacaataaaaaaaagattattaaagttaacttttacaaagaagaattaagttcacttttttatgatgtgtttatggaagaacaaagatgtttaaactatggttataaacaaccaaaaccctttgaactattatttacttatgaaaaattccaagataaaagtgtttacttaaatgcaagtactactaaaaaaaggggaagaatattttttgtttcaaaaacaaaaaataaagaagatattaataaaatttattttgataaatcaggtagatttatttttgacttcaataaagaatctgtgccttttgcaatcacactaacaaattatgataaatctaaagatattgctgaatttgatatgttgccagcttgtgagaataaccctaatcaaaattttcacttggagcacgctcattcaagtcacttctatttaaaaccaagtaataaaatattccagcatttggaattagcaattaagcataataataattcttttgttttcttaaaccctaaaaagaaatatagttttgcatatgatttaattaaccttaacttatctaagtattctaaaaatacacaaaactttatttatggaatgaatgtacatgttccagaattaattaacattaatttatcatggatgtggaaatatcaatattttaaacctaatatttttcttacatataatgacgaaaaaaatgatgtcaaaatagaacaagcaagatcaaatgaaaacaaaagtgatttatctaacttattatattgtttaaatactttaagtattttaatggttgattacaataaccactataaacttaatactgatatttttgcaatgaagaataatgcaaaagatgggaaaccatatagatggttagaatggcagaaagttaatttatcaaaatatccaaataaaaataatatgtgggttttgaaaaaatgtagatatgacaaagataatttttattggattatcagttttttcaataatgattatttatgggtgcaacaaaaaggtgaaaattggggttttttctttttagcaaataaaaaaagtaagcctttaaaatcatcttcaatattttttcttaatgaaaatcatattaagtaa

SEQ ID NO: 79 - protein, 647 aa, Mycoplasma penetrans
MINNDIKSSWLPEYYSLLENKPPKFLYRVDYKSPEEVFQKGFSINSQNEFSFFDYFFNIYSCTSTEKEDCFINAFETVNEAIINFRKNLYAINGNIKDLYLYLIRCDENFFSKHITRCTYPSALIENQVISNNKNETNKLIFAFSDYGQKFTNKFEWFTTKKISSNQVFSASHIKLNFKKNSNTKNKNDFTIIPEIEDTIFRNPNYLDLNTQANLRAFIYPEYLASKKIEFKNEAYYFEHNDKISSWMNVQNKSFDSNNIIKHRKDKPIVKQITLFDKNNKKKIIKVNFYKEELSSLFYDVFMEEQRCLNYGYKQPKPFELLFTYEKFQDKSVYLNASTTKKRGRIFFVSKTKNKEDINKIYFDKSGRFIFDFNKESVPFAITLTNYDKSKDIAEFDMLPACENNPNQNFHLEHAHSSHFYLKPSNKIFQHLELAIKHNNNSFVFLNPKKKYSFAYDLINLNLSKYSKNTQNFIYGMNVHVPELININLSWMWKYQYFKPNIFLTYNDEKNDVKIEQARSNENKSDLSNLLYCLNTLSILMVDYNNHYKLNTDIFAMKNNAKDGKPYRWLEWQKVNLSKYPNKNNMWVLKKCRYDKDNFYWIISFFNNDYLWVQQKGENWGFFFLANKKSKPLKSSSIFFLNENHIK

SEQ ID NO: 80 - protein, 9 aa, Artificial
  ADP-ribosylating toxin sequence
KFLYRVDYK

SEQ ID NO: 81 - protein, 12 aa, Artificial
  ADP-ribosylating toxin sequence
EDCFINAFETVN

SEQ ID NO: 82 - protein, 11 aa, Artificial
  ADP-ribosylating toxin sequence
KFTNKFEWFTT

SEQ ID NO: 83 - protein, 10 aa, Artificial
  ADP-ribosylating toxin sequence
VREVYRVDLR

SEQ ID NO: 84 - protein, 12 aa, Artificial
  ADP-ribosylating toxin sequence
RSYFISTSETPT

SEQ ID NO: 85 - protein, 11 aa, Artificial
  ADP-ribosylating toxin sequence
SFAYQREWFTD

SEQ ID NO: 86 - protein, 9 aa, Artificial
  ADP-ribosylating toxin sequence
ATVYRDDLR

SEQ ID NO: 87 - protein, 10 aa, Artificial
  ADP-ribosylating toxin sequence
AFVSTSSSRR

SEQ ID NO: 88 - protein, 10 aa, Artificial
  ADP-ribosylating toxin sequence
AAYQREWFAD

SEQ ID NO: 89 - protein, 9 aa, Artificial
  ADP-ribosylating toxin sequence
IVWYRYWYE

SEQ ID NO: 90 - protein, 12 aa, Artificial
  ADP-ribosylating toxin sequence
KHSFMSTTALKN

SEQ ID NO: 91 - protein, 11 aa, Artificial
  ADP-ribosylating toxin sequence
AVPSEVELLFP

SEQ ID NO: 92 - protein, 9 aa, Artificial
  ADP-ribosylating toxin sequence
VYWYRLLNL

SEQ ID NO: 93 - protein, 12 aa, Artificial
  ADP-ribosylating toxin sequence
REDGYSSTQLVS

SEQ ID NO: 94 - protein, 11 aa, Artificial
  ADP-ribosylating toxin sequence
AYYGQCEVLLP

SEQ ID NO: 95 - protein, 9 aa, Artificial
  ADP-ribosylating toxin sequence
DKLYRADSR

SEQ ID NO: 96 - protein, 12 aa, Artificial
  ADP-ribosylating toxin sequence
DDGYWSTSISLR

SEQ ID NO: 97 - protein, 11 aa, Artificial
  ADP-ribosylating toxin sequence
DHDDEQEVSAL

SEQ ID NO: 98 - protein, 9 aa, Artificial
  ADP-ribosylating toxin sequence
IMLFRCDDP

SEQ ID NO: 99 - protein, 12 aa, Artificial
  ADP-ribosylating toxin sequence
EYGYISTSLMNV

SEQ ID NO: 100 - protein, 11 aa, Artificial
  ADP-ribosylating toxin sequence
AFAGQLENLLP

SEQ ID NO: 101 - protein, 9 aa, Artificial
  ADP-ribosylating toxin sequence
VDFVYRVDS

SEQ ID NO: 102 - protein, 10 aa, Artificial
  ADP-ribosylating toxin sequence
YIATTSVNQT

SEQ ID NO: 103 - protein, 10 aa, Artificial
  ADP-ribosylating toxin sequence
RLQREYVSTL

SEQ ID NO: 104 - protein, 9 aa, Artificial
  ADP-ribosylating toxin sequence
VDFVYRVDS

SEQ ID NO: 105 - protein, 11 aa, Artificial
  ADP-ribosylating toxin sequence
YIATISDINEA

SEQ ID NO: 106 - protein, 10 aa, Artificial
  ADP-ribosylating toxin sequence
RLQSEYVSVN

SEQ ID NO: 107 - protein, 9 aa, Artificial
  ADP-ribosylating toxin sequence
EGALYRPSA

SEQ ID NO: 108 - protein, 10 aa, Artificial
  ADP-ribosylating toxin sequence
GYLGTYRSAF

SEQ ID NO: 109 - protein, 9 aa, Artificial
  ADP-ribosylating toxin sequence
PAQGEVAAM

SEQ ID NO: 110 - protein, 9 aa, Artificial
  ADP-ribosylating toxin sequence
PFALFRPSQ

SEQ ID NO: 111 - protein, 10 aa, Artificial
  ADP-ribosylating toxin sequence
GYVGTFTGRA

SEQ ID NO: 112 - protein, 9 aa, Artificial
  ADP-ribosylating toxin sequence
PRDGEVAAM

SEQ ID NO: 113 - DNA, 24 bp, Artificial
  PCR primer
accacagcagcgaaccgagaatgg

SEQ ID NO: 114 - DNA, 24 bp, Artificial
  PCR primer
gctgtgggtgtttctgaagtggaa

SEQ ID NO: 115 - DNA, 42 bp, Artificial
  PCR primer
ttttgaagtttttaatttgtaaaatttcattttttaaaaatg

SEQ ID NO: 116 - protein, 9 aa, Mycoplasma penetrans
MINNDIKSS

SEQ ID NO: 117 - protein, 236 aa, Mycoplasma penetrans
LPEYYSLLENKPPKFLYRVDYKSPEEVFQKGFSINSQNEFSFFDYFFNIYSCTSTEKEDCFINAFETVNEAIINFRKNLYAINGNIKDLYLYLIRCDENFFSKHITRCTYPSALIENQVISNNKNETNKLIFAFSDYGQKFTNKFEWFTTKKISSNQVFSASHIKLNFKKNSNTKNKNDFTIIPEIEDTIFRNPNYLDLNTQANLRAFIYPEYLASKKIEFKNEAYYFEHNDKISS

SEQ ID NO: 118 - protein, 243 aa, Mycoplasma penetrans
MNVQNKSFDSNNIIKHRKDKPIVKQITLFDKNNKKKIIKVNFYKEELSSLFYDVFMEEQRCLNYGYKQPKPFELLFTYEKFQDKSVYLNASTTKKRGRIFFVSKTKNKEDINKIYFDKSGRFIFDFNKESVPFAITLTNYDKSKDIAEFDMLPACENNPNQNFHLEHAHSSHFYLKPSNKIFQHLELAIKHNNNSFVFLNPKKKYSFAYDLINLNLSKYSKNTQNFIYGMNVHVPELININLS

SEQ ID NO: 119 - protein, 78 aa, Mycoplasma penetrans
KYQYFKPNIFLTYNDEKNDVKIEQARSNENKSDLSNLLYCLNTLSILMVDYNNHYKLNTDIFAMKNNAKDGKPYRWLE

SEQ ID NO: 120 - protein, 14 aa, Mycoplasma penetrans
QKVNLSKYPNKNNM

SEQ ID NO: 121 - protein, 32 aa, Mycoplasma penetrans
VLKKCRYDKDNFYWIISFFNNDYLWVQQKGEN

SEQ ID NO: 122 - protein, 27 aa, Mycoplasma penetrans
GFFFLANKKSKPLKSSSIFFLNENHIK

(19) United States
(12) Patent Application Publication
(10) Pub. No.: US 2009/0104185 A1
(43) Pub. Date: Apr. 23, 2009

(54) Methods and Compositions for Mycoplasma Toxins

(76) Inventors: Joel Barry Baseman, San Antonio, TX (US); Thirumalai Rengasamy Kannan, San Antonio, TX (US)

Correspondence Address: MYERS BIGEL SIBLEY & SAJOVEC, PO BOX 37428, RALEIGH, NC, 27627, US

(21) Appl. No.: 11/887,995

(22) PCT Filed: Mar. 31, 2006
(86) PCT No.: PCT/US06/12266

§ 371 Date: Nov. 25, 2008

(30) Foreign Application Priority Data
Apr. 7, 2005 (US) PCT/US2005/011897

(51) Int. Cl. A61K 39/395; C07K 16/18; C07K 14/00; C12N 15/11; G01N 33/569; C12Q 1/68; G01N 33/566; A61K 31/7088; C12Q 1/02

(52) U.S. Cl. 424/133.1; 530/387.7; 530/387.1; 530/387.3; 530/350; 536/23.7; 530/387.9; 435/7.32; 435/6; 436/501; 424/190.1; 424/184.1; 514/44; 435/29

(57) ABSTRACT

The present invention provides Mycoplasma toxins, biologically active fragments/domains of the toxins, antibodies to the toxins, therapeutic fusion proteins comprising the toxins and/or biologically active fragments/domains of the toxins and nucleic acids encoding the toxins and fusion proteins. Also provided are methods of treating and/or preventing diseases and disorders using the compositions provided herein.

### RELATED APPLICATIONS

This application claims priority to PCT Application Serial No. PCT/US2005/011897, filed Apr. 7, 2005, the entire contents of which are incorporated by reference herein.

### STATEMENT OF GOVERNMENT SUPPORT

Research related to this invention was supported, at least in part, by U.S. Government Grant No. AI45737 awarded by the NIAID. The Government has certain rights in this invention.

### BACKGROUND OF THE INVENTION

1. Field of the Invention

The present invention relates to Mycoplasma pneumoniae and Mycoplasma penetrans toxins, antibodies thereto, and their use in diagnostic and therapeutic methods.

2. Background Art

The importance of Mycoplasma pneumoniae as a causative agent of acute and chronic human respiratory diseases has been well documented by clinical and epidemiological studies in many settings. The importance of Mycoplasma pneumoniae as a cause of human respiratory disease has been well documented by epidemiological studies in various settings and in many countries. M. pneumoniae is the etiologic agent of primary atypical pneumonia and is also responsible for many respiratory tract infections, such as tracheobronchitis, bronchiolitis, pharyngitis and croup, especially in older children and young adults and in elderly populations. It accounts for 20-30% of all pneumonias and also is linked to asthmia and chronic obstructive pulmonary disease. M. pneunoniae is among the smallest self-replicating cells (816 kb genome) and is spread by direct contact or aerosol among individuals, usually young children and adolescents, although the age spectrum for susceptibility includes adults and the elderly. Large, sustained outbreaks of M. pneumoniae have occurred in closed and semi-closed populations, such as child care centers, college dormitories, hospitals, psychiatric institutions, military and religious communities, and prisons. Furthermore, M. pneumoniae can disseminate to other organ sites and cause gastrointestinal, hematologic, neurologic, dermatologic, musculoskeletal and cardiovascular pathologies. This secondary involvement by M. pneumoniae leads to a spectrum of complicated extrapulmonary sequelae, including arthritis, pericarditis and central nervous system disorders, which attests to the significance of M. pneumoniae in human disease. Definitive diagnosis and therapeutic decisions concerning M. pneumoniae are often delayed or lacking because of the relatively long incubation period (average 1-2 weeks) before clinical symptoms can be observed and before M. pneumoniae broth or colony growth can be visualized. Although antibiotic therapy appears to be relatively effective in controlling mycoplasma pneumonia, the bacteria continue to persist. At present, no known virulence determinants of M. pneumoniae have been functionally identified and linked to the wide range of pathologies associated with M. pneumoniae mediated diseases. Furthermore, there are no specific and standardized diagnostic tests available for reliable and rapid detection of M. pneumoniae infection, or effective vaccines to control M. pneumoniae infection. Mycoplasma penetrans, a member of the Mollicute (includes mycoplasmas) family, was first isolated in 1992 from the urine of HIV-infected homosexual males and is associated with the progression of AIDS. For example, 40% of HIV-infected individuals with AIDS exhibit high antibody titers against M. penetrans, while 20% of HIV-infected individuals who have not progressed to AIDS exhibit high-titered antibodies to M. penetrans. In contrast, antibodies against M. penetrans are rarely found in heterosexual and non-HIV infected groups (Giron et al. 1996. “Adherence, fibronectin binding, and induction of cytoskeleton reorganization in cultured human cells by Mycoplasma penetrans” Infect Immun 64(1):197-208). Therefore, M. penetrans is implicated in the deterioration of the immune system in AIDS patients and thought to be involved in the rapid decline of CD4+ lymphocytes. Although M. penetrans is mainly considered a urogenital tract pathogen, it has been infrequently isolated from non-HIV-infected patients with urethritis and respiratory disease manifestations.

The present invention overcomes previous shortcomings in the art by providing a Mycoplasma pneumoniae polypeptide and biologically active fragments thereof, known as community acquired respiratory distress syndrome (CARDS) toxin, as well as nucleic acids encoding this polypeptide and its fragments and antibodies specific thereto. Also provided is a Mycoplasma penetrans polypeptide and biologically active fragments thereof, as well as nucleic acids encoding this polypeptide and antibodies specific thereto. These compositions are used, for example, in methods of diagnosing, treating and preventing infection by M. pneumoniae and M. penetrans.

Some Sequences of this Invention:

Reference amino acid sequence M129/B9 (reference
strain): (SEQ ID NO: 1)
MPNPVRFVYR VDLRSPEEIF EHGFSTLGDV RNFFEHILST
NFGRSYFIST SETPTAAIRF FGSWLREYVP EHPRRAYLYE
IRADQHFYNA RATGENLLDL MRQRQVVFDS GDREMAQMGI
RALRTSFAYQ REWFTDGPIA AANVRSAWLV DAVPVEPGHA
HHPAGRVVET TRINEPEMHN PHYQELQTQA NDQPWLPTPG
IATPVHLSIP QAASVADVSE GTSASLSFAC PDWSPPSSNG
ENPLDKCIAE KIDNYNLQSL PQYASSVKEL EDTPVYLRGI
KTQKTFMLQA DPQNNNVFLV EVNPKQKSSF PQTIFFWDVY
QRICLKDLTG AQISLSLTAF TTQYAGQLKV HLSVSAVNAV
NQKWKMTPQD IAITQFRVSS ELLGQTENGL FWNTKSGGSQ
HDLYVCPLKN PPSDLEELQI IVDECTTHAQ FVTMRAASTF
FVDVQLGWYW RGYYYTPQLS GWSYQMKTPD GQIFYDLKTS
KIFFVQDNQN VFFLHNKLNK QTGYSWDWVE WLKHDMNEDK
DENFKWYFSR DDLTIPSVEG LNFRHIRCYA DNQQLKVIIS
GSRWGGWYST YDKVESNVED KILVKDGFDR F

Trypsin digestion of rCARDS TX (rMPN372) yielded two different distinct and stable domains. The amino acid (aa) sequence from aa 1-307 is ˜37 kDa and represents the N-terminal part of the toxin domain; this sequence encodes the ADP ribosylation region and likely represents the catalytic activity of the toxin. The remaining aa sequence from 308-591 is ˜34 kDa and represents the C-terminal part. Attached is the CARDS TX sequence with identified trypsin cleavage site labeled in red. One of the key points is that the C-terminal domain (308-591) probably represents the “B” domain of toxins like diphtheria toxin, which is likely involved in binding of CARDS TX to specific host targets. Because it I trypsin-resistant, it would be a prime candidate for diagnostic, vaccine and anti-drug targets.

Calculated MW:
N domain: HisTag+res1-307=37.3 kDa
C-domain: res308-591=33.2 kDa

Fragment 2 (aa 308-591) is trypsin resistant.

Putative transmembrane domains of toxin
Potential transmembrane segments

| Start | Stop | Length | ~ | Cutoff |
|---|---|---|---|---|
| 333 | 339 | 7 | ~ | 1.7 |
| 440 | 441 | 2 | ~ | 1.7 |

S1 (clinical strain) amino acid sequence:
(SEQ ID NO: 2)
MPNPVRFVYRVDLRSPEEIFEHGFSTLGDVRNFFEHIPSTNFGRSYFIST
SETPTAAIRF
FGSWLREYVPEHPRRAYLYEIRADQHFYNARATGENLLDLMRQRQVVFDS
GDREMAQMGI
RALRTSFAYQREWFTDGPIAAANVRSAWLVDAVPVEPGHAHHPAGRVVET
TRINEPEMHN
PHYQELQTQANDQPWLPTPGIATPVHLSIPQAASVADVSEGTSASLSFAC
PDWSPPSSNG
ENPLDKCIAEKIDNYNLQSLPQYASSVKELEDTPVYLRGIKTQKTFMLQA
DPQNNNVFLV
EVNPKQKSPFPQTIFFWDVYQRICLKDLTGAQISLSLTAFTTQYAGQLKY
HLSYSAVNAV
NQKWKMTPQDSAITQFRVSSELLGQTENGLSWNTKSGGSQHDLYVCPLKN
PPSDLEELQI
IVDECTTHAQFVTMRAASTFFVDVQLGWYWRGYYYTPQLSGWSYQMKTPD
GQIFYDLKTS
KIFFVQDNQNVFFLHNKLNKQTGYSWDWVEWLKHDMNEDKDENFKWYFSR
DDLTIPSVEG
LNFRHIRCYADNQQLKVIISGSRWGGWYSTYDKVESNVEDKILVKDGFDR
F
JL (clinical strain) amino acid sequence:
(SEQ ID NO: 3)
MPNPVRFVYRVDLRSPEEIFEHGFSTLGDVRNFFEHILSTNFGRSYFIST
SETPTAAIRF
FGSWLREYVPEHPRRAYLYEIRADQHFYNARATGENLLDLMRQRQVVFDS
GDREMAQMGI
RALRTSFAYQREWFTDGPIAAANVRSAWLVDAVPVEPGHAHHPAGRVVET
TRINEPEMHN
PHYQELQTQANDQPWLPTPGIATPVHLSIPQAASVADVSEGTSASLSFAC
PDWSPPSSNG
ENPLDKCIAEKIDNYNLQSLPQYASSVKELEDTPVYLRGIKTQKTFMLQA
DPQNNNVFLV
EVNPKQKSSFPQTIFFWDVYQRICLKDLTGAQISLSLTAFTTQYAGQLKV
HLSVSAVNAV
NQKWKMTPQDSAITQFRVSSELLGQTENGLFWNTKSGGSQHDLYVCPLKN
PPSDLEELQI
IVDECTTHAQFVTMRAASTFFVDVQLGWYWRGYYYTPQLSGWSYQMKTPD
GQIFYDLKTS
KIFFVQDNQNVFFLHNKLNKQTGYSWDWVEWLKHDMNEDKDENFKWYFSR
DDLTIPSVEG
LNFRHIRCYADNQQLKVIISGSRWGGWYSTYDKVESNVEDKILVKDGFDR
F
RJL1 (clinical strain) amino acid sequence:
(SEQ ID NO: 4)
MPNPVRFVYRVDLRSPEEIFEHGFSTLGDVRNFFEHILSTNFGRSYFIST
SETPTAAIRF
FGSWLREYVPEHPRRAYLYEIRADQHFYNARATGENLLDLMRQRQVVFDS
GDREMAQMGI
RALRTSFAYQREWFTDGPIAAANVRSAWLVDAVPVEPGHAHHPAGRVVET
TRINEPEMHN
PHYQELQTQANDQPWLPTPGIATPVHLSIPQAASVADVSEGTSASLSFAC
PDWSPPSSNG
ENPLDKCIAEKIDNYNLQSLPQYASSVKELEDTPVYLRGIKTQKTFMLQA
DPQNNNVFLV
EVNPKQKSSFPQTIFFWDVYQRICLKDLTGAQISLSLTAFTTQYAGQLKV
HLSVSAVNAV
NQKWKMTPQDSAITQFRVSSELLGQTENGLFRNTKSGGSQHDLYVCPLKN
PPSDLEELQI
IVDECTTHAQFVTMRAASTFFVDVQLGWYWRGYYYTPQLSGWSYQMKTPD
GQIFYDLKTS
KIFFVQDNQNVFFLHNKLNKQTGYSWDWVEWLKHDMNEDKDENFKWYFSR
DDLTIPSVEG
LNFRHIRCYADNQQLKVIISGSRWGGWYSTYDKVESNVEDKILVKDGFDR
F
L2 (clinical strain) amino acid sequence:
(SEQ ID NO: 5)
MPNPVRPVYRVDLRSPEEIFEHGFSTLGDVRNFFEHILSTNFGRSYFIST
SETPTAAIRF
FGSWLREYVPEHPRRAYLYEIRADQHFYNARATGENLLDLMRQRQVVFDS
GDREMAQMGI
RALRTSFAYQREWFTDGPIAAANYRSAWLVDAVPVEPGHAHHPAGRVVET
TRINEPEMHN
PHYQELQTQANDQPWLPTPGIATPVHLSIPQAASVADVSEGTSASLSFAC
PDWSPPSSNG
ENPLGKCIAEKIDNYNLQSLPQYASSVKELEDTPVYLRGIKTQKTFMLQA
DPQNNNVFLV
EVNPKQKSSFPQTIFFWDVYQRICLKDLTGAQISLSLTAFTTQYAGQLKV
HLSVSAVNAV
NQKWKMTPQDSAITQFRVSSELLGQTENGLFWNTKSGGSQHDLYVCPLKN
PPSDLEELQI
IVDECTTHAQFVTMRAASTFFVDVQLGWYWRGYYYTPQLSGWSYQMKTPD
GQIFYDLKTS
KIFFVQDNQNVFFLHNKLNKQTGYSWDWVEWLKHDMNEDKDENFKWYFSR
DDLTIPSVEG
LNFRHIRCYADNQQLKVIISGSRWGGWYSTYDKYESNVEDKILVKDGFDR
F
Composite amino acid sequence: (SEQ ID NO: 6)
MPNPVRFVYR VDLRSPEEIF EHGFSTLGDV RNFFEHIPST
NFGRSYFIST SETPTAAIRF
FGSWLREYVP EHPRRAYLYE IRADQHFYNA RATGENLLDL
MRQRQVVFDS GDREMAQMGI
RALRTSFAYQ REWFTDGPIA AANVRSAWLV DAVPVEPGHA
HHPAGRVVET TRINEPEMHN
PHYQELQTQA NDQPWLPTPG IATPVHLSIP QAASVADVSE
GTSASLSFAC PDWSPPSSNG
ENPLGKCIAE KIDNYNLQSL PQYASSVKEL EDTPVYLRGI
KTQKTFMLQA DPQNNNVFLV
EVNPKQKPSF PQTIFFWDVY QRICLKDLTG AQISLSLTAF
TTQYAGQLKV HLSVSAVNAV
NQKWKMTPQD SAITQFRVSS ELLGQTENGL SRNTKSGGSQ
HDLYVCPLKN PPSDLEELQI
IVDECTTHAQ FVTMRAASTF FVDYQLGWYW RGYYYTPQLS
GWSYQMKTPD GQIFYDLKTS
KIFFVQDNQN VFFLHNKLNK QTGYSWDWVE WLKHDMNEDK
DENFKWYFSR DDLTIPSVEG
LNFRHIRCYA DNQQLKVIIS GSRWGGWYST YDKVESNVED
KILVKDGFDR F
Amino acid sequence of M. penetrans toxin
(SEQ ID NO: 79)
MINDIKSSWLPEYYSLLENKPPKFLYRVDYKSPEEVFQKGFSINSQNEFS
FFDYFFNIYSCTSTEKEDCFINAFETVNEAIINFRKNLYAINGNIKDLYL
YLIRCDENFFSKHITRCTYPSALIENQVISNNKNETNKLIFAFSDYGQKF
TNKFEWFTTKKISSNQVFSASHIKLNFKKNSNTKNKNDFTIIPEIEDTIF
RNPNYLDLNTQANLRAFIYPEYLASKKIEFKNEAYYFEHNDKISSWMNVQ
NKSFDSNNIIKHRKDKPIVKQITLFDKNNKKKIIKVNFYKEELSSLFYDV
FMEEQRCLNYGYKQPRPFELLFTYEKFQDKSVYLNASTTKKRGRIFFVSK
TKNKEDINKIYFDKSGRFIFDFNKESVPFAITLTNYDKSKDIAEFDMLPA
CENNPNQNFHLEHAHSSHFYLKPSNKIFQHLELAIKHNNNSFVFLNPKKK
YSFAYDLINLNLSKYSKNTQNFIYGMNVHVPELININLSWMWKYQYFKPN
IFLTYNDEKNDVKIEQARSNENKSDLSNLLYCLNTLSILMVDYNNHYKLN
TDIFAMKNNAKDGKPYRWLEWQKVNLSKYPNKNNMWVLKKCRYDKDNFYW
IISFFNNDYLWVQQKGENWGFFFLANKKSKPLKSSSIFFLNENHIK

| Reference nucleotide sequence M129/B9 (contains tga codons that | |
|---|---|
| need to be changed to tgg before expression in E. coli) | |
| (SEQ ID NO: 7) | |
| tttttaattt gtaaaatttc attttttaaa aatgccaaat cctgttagat ttgtttaccg | |
| tgttgatttg agaagccctg aagaaatttt tgaacatggc ttttcaactt taggtgatgt | |
| gagaaatttc tttgaacaca ttctctccac taattttggt agaagctatt ttatttccac | |
| ttcagaaaca cccacagcag ctattcgctt ctttggtagc tggttacggg aatatgtacc | |
| agagcacccc agaagggctt acttatatga aattcgtgcc gaccaacact tttacaatgc | |
| ccgcgccact ggggagaact tgttagattt aatgcgtcaa agacaagtag tatttgactc | |
| tggtgatcga gaaatggcac aaatgggaat tagagcttta cgcacttcct ttgcgtatca | |
| acgtgaatgg tttaccgatg gtccaattgc agcagctaat gtccgtagtg cttgactagt | |
| agatgctgtt cccgttgaac ctggtcatgc tcaccacccg gctggtcgtg ttgtagagac | |
| tactagaatt aatgaaccgg aaatgcacaa ccctcattat caagagctgc aaacccaagc | |
| caatgatcaa ccatgattgc caacaccagg aatagctact cctgtacatt tatcaattcc | |
| ccaagcagct tccgttgctg atgtttcgga aggtacttcc gcttcgctat cgtttgcgtg | |
| ccctgattga agtccacctt ctagtaatgg tgaaaatccg ctagacaaat gcattgcgga | |
| aaagattgat aactataacc tacaatcctt accacagtac gctagcagtg taaaggaact | |
| ggaagataca ccagtatacc taaggggaat taaaacgcaa aaaaccttta tgttacaagc | |
| agatccgcaa aataacaatg tctttttggt cgaagtaaac cccaaacaaa agtccagctt | |
| tccccaaacc atcttctttt gggatgttta tcaacgaatt tgtctcaagg atttaactgg | |
| tgcacaaatc agtctttcgc ttactgcctt tactactcag tatgctggtc agctcaaagt | |
| gcaccttagt gttagcgcgg ttaatgccgt gaaccaaaag tgaaaaatga caccgcaaga | |
| cattgcaata actcagtttc gggtctcctc tgaactgtta ggtcaaactg aaaatggctt | |
| gttctgaaat accaagagtg gtggttcaca acacgatttg tatgtatgtc ctttgaaaaa | |
| tccacctagt gatttggaag aattacaaat aattgttgat gaatgtacta cccatgcgca | |
| gtttgttact atgcgtgcag ctagcacctt ctttgttgat gttcagctag gctggtattg | |
| aaggggttat tactataccc cacaattaag tggttgatct tatcagatga aaacaccaga | |
| tggacagata ttctatgatc taaaaacttc gaaaatcttc tttgtccagg acaaccaaaa | |
| cgtgttcttt ctccataata aactcaacaa acaaactggt tacagctggg attgagtaga | |
| atggctaaaa catgacatga atgaggacaa agacgaaaac tttaaatggt acttttcgcg | |
| tgatgacctt accattcctt ccgttgaagg gcttaacttc cgccacattc gctgttacgc | |
| tgacaaccag cagttaaagg tgatcataag cggttcacgt tggggcggtt ggtactccac | |
| ttacgataaa gttgaaagta atgtcgaaga taagattttg gtcaaagatg gttttgatcg | |
| cttttagcga ttaagcttta acgtcactgt tttgctctaa tgttagaagc aaagatcttg | |
| S1 Nucleotide sequence with each tga changed to tgg for | |
| expression in E. coli (SEQ ID NO: 8) | |
| atgccaaatc ctgttagatt tgtttaccgt gttgatttga gaagccctga agaaattttt | 60 |
| gaacatggct tttcaacttt aggtgatgtg agaaatttct ttgaacacat tccctccact | 120 |
| aattttggta gaagctattt tatttccact tcagaaacac ccacagcagc tattcgcttc | 180 |
| tttggtagct ggttacggga atatgtacca gagcacccca gaagggctta cttatatgaa | 240 |
| attcgtgccg accaacactt ttacaatgcc cgcgccactg gggagaactt gttagattta | 300 |
| atgcgtcaaa gacaagtagt atttgactct ggtgatcgag aaatggcaca aatgggaatt | 360 |
| agagctttac gcacttcctt tgcgtatcaa cgtgaatggt ttaccgatgg tccaattgca | 420 |
| gcagctaatg tccgtagtgc ttggctagta gatgctgttc ccgttgaacc tggtcatgct | 480 |
| caccacccgg ctggtcgtgt tgtagagact actagaatta atgaaccgga aatgcacaac | 540 |
| cctcattatc aagagctgca aacccaagcc aatgatcaac catggttgcc aacaccagga | 600 |
| atagctactc ctgtacattt atcaattccc caagcagctt ccgttgctga tgtttcggaa | 660 |
| ggtacttccg cttcgctatc gtttgcgtgc cctgattgga gtccaccttc tagtaatggt | 720 |
| gaaaatccgc tagacaaatg cattgcggaa aagattgata actataacct acaatcctta | 780 |
| ccacagtacg ctagcagtgt aaaggaactg gaagatacac cagtatacct aaggggaatt | 840 |
| aaaacgcaaa aaacctttat gttacaagca gatccgcaaa ataacaatgt ctttttggtc | 900 |
| gaagtaaacc ccaaacaaaa gcccagcttt ccccaaacca tcttcttttg ggatgtttat | 960 |
| caacgaattt gtctcaagga tttaactggt gcacaaatca gtctttcgct tactgccttt | 1020 |
| actactcagt atgctggtca gctcaaagtg caccttagtg ttagcgcggt taatgccgtg | 1080 |
| aaccaaaagt ggaaaatgac accgcaagac agtgcaataa ctcagtttcg ggtctcctct | 1140 |
| gaactgttag gtcaaactga aaatggcttg tcctggaata ccaagagtgg tggttcacaa | 1200 |
| cacgatttgt atgtatgtcc tttgaaaaat ccacctagtg atttggaaga attacaaata | 1260 |
| attgttgatg aatgtactac ccatgcgcag tttgttacta tgcgtgcagc tagcaccttc | 1320 |
| tttgttgatg ttcagctagg ctggtattgg aggggttatt actatacccc acaattaagt | 1380 |
| ggttggtctt atcagatgaa aacaccagat ggacagatat tctatgatct aaaaacttcg | 1440 |
| aaaatcttct ttgtccagga caaccaaaac gtgttctttc tccataataa actcaacaaa | 1500 |
| caaactggtt acagctggga ttgggtagaa tggctaaaac atgacatgaa tgaggacaaa | 1560 |
| gacgaaaact ttaaatggta cttttcgcgt gatgacctta ccattccttc cgttgaaggg | 1620 |
| cttaacttcc gccacattcg ctgttacgct gacaaccagc agttaaaggt gatcataagc | 1680 |
| ggttcacgtt ggggcggttg gtactccact tacgataaag ttgaaagtaa tgtcgaagat | 1740 |
| aagattttgg tcaaagatgg ttttgatcgc ttt | 1773 |
| L2 nucleotide sequence with each tga changed to tgg for | |
| expression in E. coli (SEQ ID NO: 9) | |
| atgccaaatc ctgttagatt tgtttaccgt gttgatttga gaagccctga agaaattttt | 60 |
| gaacatggct tttcaacttt aggtgatgtg agaaatttct ttgaacacat tctctccact | 120 |
| aattttggta gaagctattt tatttccact tcagaaacac ccacagcagc tattcgcttc | 180 |
| tttggtagct ggttacggga atatgtacca gagcacccca gaagggctta cttatatgaa | 240 |
| attcgtgccg accaacactt ttacaatgcc cgcgccactg gggagaactt gttagattta | 300 |
| atgcgtcaaa gacaagtagt atttgactct ggtgatcgag aaatggcaca aatgggaatt | 360 |
| agagctttac gcacttcctt tgcgtatcaa cgtgaatggt ttaccgatgg tccaattgca | 420 |
| gcagctaatg tccgtagtgc ttggctagta gatgctgttc ccgttgaacc tggtcatgct | 480 |
| caccacccgg ctggtcgtgt tgtagagact actagaatta atgaaccgga aatgcacaac | 540 |
| cctcattatc aagagctgca aacccaagcc aatgatcaac catggttgcc aacaccagga | 600 |
| atagctactc ctgtacattt atcaattccc caagcagctt ccgttgctga tgtttcggaa | 660 |
| ggtacttccg cttcgctatc gtttgcgtgc cctgattgga gtccaccttc tagtaatggt | 720 |
| gaaaatccgc taggcaaatg cattgcggaa aagattgata actataacct acaatcctta | 780 |
| ccacagtacg ctagcagtgt aaaggaactg gaagatacac cagtatacct aaggggaatt | 840 |
| aaaacgcaaa aaacctttat gttacaagca gatccgcaaa ataacaatgt ctttttggtc | 900 |
| gaagtaaacc ccaaacaaaa gtccagcttt ccccaaacca tcttcttttg ggatgtttat | 960 |
| caacgaattt gtctcaagga tttaactggt gcacaaatca gtctttcgct tactgccttt | 1020 |
| actactcagt atgctggtca gctcaaagtg caccttagtg ttagcgcggt taatgccgtg | 1080 |
| aaccaaaagt ggaaaatgac accgcaagac agtgcaataa ctcagtttcg ggtctcctct | 1140 |
| gaactgttag gtcaaactga aaatggcttg ttctggaata ccaagagtgg tggttcacaa | 1200 |
| cacgatttgt atgtatgtcc tttgaaaaat ccacctagtg atttggaaga attacaaata | 1260 |
| attgttgatg aatgtactac ccatgcgcag tttgttacta tgcgtgcagc tagcaccttc | 1320 |
| tttgttgatg ttcagctagg ctggtattgg aggggttatt actatacccc acaattaagt | 1380 |
| ggttggtctt atcagatgaa aacaccagat ggacagatat tctatgatct aaaaacttcg | 1440 |
| aaaatcttct ttgtccagga caaccaaaac gtgttctttc tccataataa actcaacaaa | 1500 |
| caaactggtt acagctggga ttgggtagaa tggctaaaac atgacatgaa tgaggacaaa | 1560 |
| gacgaaaact ttaaatggta cttttcgcgt gatgacctta ccattccttc cgttgaaggg | 1620 |
| cttaacttcc gccacattcg ctgttacgct gacaaccagc agttaaaggt gatcataagc | 1680 |
| ggttcacgtt ggggcggttg gtactccact tacgataaag ttgaaagtaa tgtcgaagat | 1740 |
| aagattttgg tcaaagatgg ttttgatcgc ttt | 1773 |
| JL nucleotide sequence with each tga changed to tgg for | |
| expression in E. coli (SEQ ID NO: 10) | |
| atgccaaatc ctgttagatt tgtttaccgt gttgatttga gaagccctga agaaattttt | 60 |
| gaacatggct tttcaacttt aggtgatgtg agaaatttct ttgaacacat tctctccact | 120 |
| aattttggta gaagctattt tatttccact tcagaaacac ccacagcagc tattcgcttc | 180 |
| tttggtagct ggttacggga atatgtacca gagcacccca gaagggctta cttatatgaa | 240 |
| attcgtgccg accaacactt ttacaatgcc cgcgccactg gggagaactt gttagattta | 300 |
| atgcgtcaaa gacaagtagt atttgactct ggtgatcgag aaatggcaca aatgggaatt | 360 |
| agagctttac gcacttcctt tgcgtatcaa cgtgaatggt ttaccgatgg tccaattgca | 420 |
| gcagctaatg tccgtagtgc ttggctagta gatgctgttc ccgttgaacc tggtcatgct | 480 |
| caccacccgg ctggtcgtgt tgtagagact actagaatta atgaaccgga aatgcacaac | 540 |
| cctcattatc aagagctgca aacccaagcc aatgatcaac catggttgcc aacaccagga | 600 |
| atagctactc ctgtacattt atcaattccc caagcagctt ccgttgctga tgtttcggaa | 660 |
| ggtacttccg cttcgctatc gtttgcgtgc cctgattgga gtccaccttc tagtaatggt | 720 |
| gaaaatccgc tagacaaatg cattgcggaa aagattgata actataacct acaatcctta | 780 |
| ccacagtacg ctagcagtgt aaaggaactg gaagatacac cagtatacct aaggggaatt | 840 |
| aaaacgcaaa aaacctttat gttacaagca gatccgcaaa ataacaatgt ctttttggtc | 900 |
| gaagtaaacc ccaaacaaaa gtccagcttt ccccaaacca tcttcttttg ggatgtttat | 960 |
| caacgaattt gtctcaagga tttaactggt gcacaaatca gtctttcgct tactgccttt | 1020 |
| actactcagt atgctggtca gctcaaagtg caccttagtg ttagcgcggt taatgccgtg | 1080 |
| aaccaaaagt ggaaaatgac accgcaagac agtgcaataa ctcagtttcg ggtctcctct | 1140 |
| gaactgttag gtcaaactga aaatggcttg ttctggaata ccaagagtgg tggttcacaa | 1200 |
| cacgatttgt atgtatgtcc tttgaaaaat ccacctagtg atttggaaga attacaaata | 1260 |
| attgttgatg aatgtactac ccatgcgcag tttgttacta tgcgtgcagc tagcaccttc | 1320 |
| tttgttgatg ttcagctagg ctggtattgg aggggttatt actatacccc acaattaagt | 1380 |
| ggttggtctt atcagatgaa aacaccagat ggacagatat tctatgatct aaaaacttcg | 1440 |
| aaaatcttct ttgtccagga caaccaaaac gtgttctttc tccataataa actcaacaaa | 1500 |
| caaactggtt acagctggga ttgggtagaa tggctaaaac atgacatgaa tgaggacaaa | 1560 |
| gacgaaaact ttaaatggta cttttcgcgt gatgacctta ccattccttc cgttgaaggg | 1620 |
| cttaacttcc gccacattcg ctgttacgct gacaaccagc agttaaaggt gatcataagc | 1680 |
| ggttcacgtt ggggcggttg gtactccact tacgataaag ttgaaagtaa tgtcgaagat | 1740 |
| aagattttgg tcaaagatgg ttttgatcgc ttt | 1773 |
| RJL1 nucleotide sequence with each tga changed to tgg for | |
| expression in E. coli (SEQ ID NO: 11) | |
| atgccaaatc ctgttagatt tgtttaccgt gttgatttga gaagccctga agaaattttt | 60 |
| gaacatggct tttcaacttt aggtgatgtg agaaatttct ttgaacacat tctctccact | 120 |
| aattttggta gaagctattt tatttccact tcagaaacac ccacagcagc tattcgcttc | 180 |
| tttggtagct ggttacggga atatgtacca gagcacccca gaagggctta cttatatgaa | 240 |
| attcgtgccg accaacactt ttacaatgcc cgcgccactg gggagaactt gttagattta | 300 |
| atgcgtcaaa gacaagtagt atttgactct ggtgatcgag aaatggcaca aatgggaatt | 360 |
| agagctttac gcacttcctt tgcgtatcaa cgtgaatggt ttaccgatgg tccaattgca | 420 |
| gcagctaatg tccgtagtgc ttggctagta gatgctgttc ccgttgaacc tggtcatgct | 480 |
| caccacccgg ctggtcgtgt tgtagagact actagaatta atgaaccgga aatgcacaac | 540 |
| cctcattatc aagagctgca aacccaagcc aatgatcaac catggttgcc aacaccagga | 600 |
| atagctactc ctgtacattt atcaattccc caagcagctt ccgttgctga tgtttcggaa | 660 |
| ggtacttccg cttcgctatc gtttgcgtgc cctgattgga gtccaccttc tagtaatggt | 720 |
| gaaaatccgc tagacaaatg cattgcggaa aagattgata actataacct acaatcctta | 780 |
| ccacagtacg ctagcagtgt aaaggaactg gaagatacac cagtatacct aaggggaatt | 840 |
| aaaacgcaaa aaacctttat gttacaagca gatccgcaaa ataacaatgt ctttttggtc | 900 |
| gaagtaaacc ccaaacaaaa gtccagcttt ccccaaacca tcttcttttg ggatgtttat | 960 |
| caacgaattt gtctcaagga tttaactggt gcacaaatca gtctttcgct tactgccttt | 1020 |
| actactcagt atgctggtca gctcaaagtg caccttagtg ttagcgcggt taatgccgtg | 1080 |
| aaccaaaagt ggaaaatgac accgcaagac agtgcaataa ctcagtttcg ggtctcctct | 1140 |
| gaactgttag gtcaaactga aaatggcttg ttccggaata ccaagagtgg tggttcacaa | 1200 |
| cacgatttgt atgtatgtcc tttgaaaaat ccacctagtg atttggaaga attacaaata | 1260 |
| attgttgatg aatgtactac ccatgcgcag tttgttacta tgcgtgcagc tagcaccttc | 1320 |
| tttgttgatg ttcagctagg ctggtattgg aggggttatt actatacccc acaattaagt | 1380 |
| ggttggtctt atcagatgaa aacaccagat ggacagatat tctatgatct aaaaacttcg | 1440 |
| aaaatcttct ttgtccagga caaccaaaac gtgttctttc tccataataa actcaacaaa | 1500 |
| caaactggtt acagctggga ttgggtagaa tggctaaaac atgacatgaa tgaggacaaa | 1560 |
| gacgaaaact ttaaatggta cttttcgcgt gatgacctta ccattccttc cgttgaaggg | 1620 |
| cttaacttcc gccacattcg ctgttacgct gacaaccagc agttaaaggt gatcataagc | 1680 |
| ggttcacgtt ggggcggttg gtactccact tacgataaag ttgaaagtaa tgtcgaagat | 1740 |
| aagattttgg tcaaagatgg ttttgatcgc ttt | 1773 |

Nucleotide sequence of M. penetrans toxin
(unmodified) (SEQ ID NO: 77)
ATGATTAACAATGATATTAAATCATCTTGATTACCTGAATATTATTCATT
ATTAGAAAATAAACCACCAAAATTTTTATATAGAGTTGATTACAAAAGCC
CTGAAGAAGTTTTTCAAAAAGGATTTTCAATTAATAGTCAAAATGAGTTT
AGTTTTTTTGACTATTTTTTTAATATTTATTCTTGTACTAGTACTGAAAA
AGAAGATTGTTTCATTAATGCTTTTGAAACAGTAAATGAAGCAATAATTA
ATTTTAGAAAAAATTTATATGCAATCAATGGAAATATAAAAGATCTATAT
TTGTATTTGATTAGATGTGATGAAAATTTTTTCAGTAAACATATAACTAG
ATGTACATATCCTTCTGCACTTATTGAAAACCAAGTTATATCAAATAACA
AAAATGAAACAAATAAGTTAATTTTTGCATTTAGTGATTATGGTCAAAAA
TTTACTAATAAATTTGAATGGTTCACAACTAAAAAAATTAGTAGTAACCA
AGTGTTTTCTGCTTCGCATATTAAATTAAATTTTAAAAAGAATTCAAATA
CTAAAAATAAAAATGACTTTACAATAATCCCGGAAATTGAAGATACTATC
TTTAGAAATCCTAATTATTTAGATTTGAATACTCAAGCAAATTTAAGGGC
TTTTATATATCCTGAATATTTAGCTTCAAAAAAAATTGAATTTAAAAATG
AAGCTTATTATTTTGAACACAATGACAAAATATCTTCATGAATGAATGTT
CAAAATAAATCATTTGATAGTAACAATATTATTAAACACAGAAAAGATAA
ACCAATTGTTAAACAAATTACTTTGTTTGATAAAAACAATAAAAAAAAGA
TTATTAAAGTTAACTTTTACAAAGAAGAATTAAGTTCACTTTTTTATGAT
GTGTTTATGGAAGAACAAAGATGTTTAAACTATGGTTATAAACAACCAAA
ACCCTTTGAACTATTATTTACTTATGAAAAATTCCAAGATAAAAGTGTTT
ACTTAAATGCAAGTACTACTAAAAAAAGGGGAAGAATATTTTTTGTTTCA
AAAACAAAAAATAAAGAAGATATTAATAAAATTTATTTTGATAAATCAGG
TAGATTTATTTTTGACTTCAATAAAGAATCTGTGCCTTTTGCAATCACAC
TAACAAATTATGATAAATCTAAAGATATTGCTGAATTTGATATGTTGCCA
GCTTGTGAGAATAACCCTAATCAAAATTTTCACTTGGAGCACGCTCATTC
AAGTCACTTCTATTTAAAACCAAGTAATAAAATATTCCAGCATTTGGAAT
TAGCAATTAAGCATAATAATAATTCTTTTGTTTTCTTAAACCCTAAAAAG
AAATATAGTTTTGCATATGATTTAATTAACCTTAACTTATCTAAGTATTC
TAAAAATACACAAAACTTTATTTATGGAATGAATGTACATGTTCCAGAAT
TAATTAACATTAATTTATCATGAATGTGAAAATATCAATATTTTAAACCT
AATATTTTTCTTACATATAATGACGAAAAAAATGATGTCAAAATAGAACA
AGCAAGATCAAATGAAAACAAAAGTGATTTATCTAACTTATTATATTGTT
TAAATACTTTAAGTATTTTAATGGTTGATTACAATAACCACTATAAACTT
AATACTGATATTTTTGCAATGAAGAATAATGCAAAAGATGGGAAACCATA
TAGATGGTTAGAATGACAGAAAGTTAATTTATCAAAATATCCAAATAAAA
ATAATATGTGAGTTTTGAAAAAATGTAGATATGACAAAGATAATTTTTAT
TGGATTATCAGTTTTTTCAATAATGATTATTTATGGGTGCAACAAAAAGG
TGAAAATTGAGGTTTTTTCTTTTTAGCAAATAAAAAAAGTAAGCCTTTAA
AATCATCTTCAATATTTTTTCTTAATGAAAATCATATTAAGTAA

```
Normal translation of M. penetrans sequence
(SEQ ID NOs: 116-122)
atgattaacaatgatattaaatcatcttgattacctgaatattattcattattagaaaat
  M I N N D I K S S - L P E Y Y S L L E N
aaaccaccaaaatttttatatagagttgattacaaaagccctgaagaagtttttcaaaaa
  K P P K F L Y R V D Y K S P E E V F Q K
ggattttcaattaatagtcaaaatgagtttagtttttttgactatttttttaatatttat
  G F S I N S Q N E F S F F D Y F F N I Y
tcttgtactagtactgaaaaagaagattgtttcattaatgcttttgaaacagtaaatgaa
  S C T S T E K E D C F I N A F E T V N E
gcaataattaattttagaaaaaatttatatgcaatcaatggaaatataaaagatctatat
  A I I N F R K N L Y A I N G N I K D L Y
ttgtatttgattagatgtgatgaaaattttttcagtaaacatataactagatgtacatat
  L Y L I R C D E N F F S K H I T R C T Y
ccttctgcacttattgaaaaccaagttatatcaaataacaaaaatgaaacaaataagtta
  P S A L I E N Q V I S N N K N E T N K L
atttttgcatttagtgattatggtcaaaaatttactaataaatttgaatggttcacaact
  I F A F S D Y G Q K F T N K F E W F T T
aaaaaaattagtagtaaccaagtgttttctgcttcgcatattaaattaaattttaaaaag
  K K I S S N Q V F S A S H I K L N F K K
aattcaaatactaaaaataaaaatgactttacaataatcccggaaattgaagatactatc
  N S N T K N K N D F T I I P E I E D T I
tttagaaatcctaattatttagatttgaatactcaagcaaatttaagggcttttatatat
  F R N P N Y L D L N T Q A N L R A F I Y
cctgaatatttagcttcaaaaaaaattgaatttaaaaatgaagcttattattttgaacac
  P E Y L A S K K I E F K N E A Y Y F E H
aatgacaaaatatcttcatgaatgaatgttcaaaataaatcatttgatagtaacaatatt
  N D K I S S - M N V Q N K S F D S N N I
attaaacacagaaaagataaaccaattgttaaacaaattactttgtttgataaaaacaat
  I K H R K D K P I V K Q I T L F D K N N
aaaaaaaagattattaaagttaacttttacaaagaagaattaagttcacttttttatgat
  K K K I I K V N F Y K E E L S S L F Y D
gtgtttatggaagaacaaagatgtttaaactatggttataaacaaccaaaaccctttgaa
  V F M E E Q R C L N Y G Y K Q P K P F E
ctattatttacttatgaaaaattccaagataaaagtgtttacttaaatgcaagtactact
  L L F T Y E K F Q D K S V Y L N A S T T
aaaaaaaggggaagaatattttttgtttcaaaaacaaaaaataaagaagatattaataaa
  K K R G R I F F V S K T K N K E D I N K
atttattttgataaatcaggtagatttatttttgacttcaataaagaatctgtgcctttt
  I Y F D K S G R F I F D F N K E S V P F
gcaatcacactaacaaattatgataaatctaaagatattgctgaatttgatatgttgcca
  A I T L T N Y D K S K D I A E F D M L P
gcttgtgagaataaccctaatcaaaattttcacttggagcacgctcattcaagtcacttc
  A C E N N P N Q N F H L E H A H S S H F
tatttaaaaccaagtaataaaatattccagcatttggaattagcaattaagcataataat
  Y L K P S N K I F Q H L E L A I K H N N
aattcttttgttttcttaaaccctaaaaagaaatatagttttgcatatgatttaattaac
  N S F V F L N P K K K Y S F A Y D L I N
cttaacttatctaagtattctaaaaatacacaaaactttatttatggaatgaatgtacat
  L N L S K Y S K N T Q N F I Y G M N V H
gttccagaattaattaacattaatttatcatgaatgtgaaaatatcaatattttaaacct
  V P E L I N I N L S - M - K Y Q Y F K P
aatatttttcttacatataatgacgaaaaaaatgatgtcaaaatagaacaagcaagatca
  N I F L T Y N D E K N D V K I E Q A R S
aatgaaaacaaaagtgatttatctaacttattatattgtttaaatactttaagtatttta
  N E N K S D L S N L L Y C L N T L S I L
atggttgattacaataaccactataaacttaatactgatatttttgcaatgaagaataat
  M V D Y N N H Y K L N T D I F A M K N N
gcaaaagatgggaaaccatatagatggttagaatgacagaaagttaatttatcaaaatat
  A K D G K P Y R W L E - Q K V N L S K Y
ccaaataaaaataatatgtgagttttgaaaaaatgtagatatgacaaagataatttttat
  P N K N N M - V L K K C R Y D K D N F Y
tggattatcagttttttcaataatgattatttatgggtgcaacaaaaaggtgaaaattga
  W I I S F F N N D Y L W V Q Q K G E N -
ggttttttctttttagcaaataaaaaaagtaagcctttaaaatcatcttcaatatttttt
  G F F F L A N K K S K P L K S S S I F F
cttaatgaaaatcatattaagtaa
  L N E N H I K -
```

Nucleotide sequence of M. penetrans toxin with ugg
modifications (SEQ ID NO: 78)
atgattaacaatgatattaaatcatcttggttacctgaatattattcattattagaaaat
aaaccaccaaaatttttatatagagttgattacaaaagccctgaagaagtttttcaaaaa
ggattttcaattaatagtcaaaatgagtttagtttttttgactatttttttaatatttat
tcttgtactagtactgaaaaagaagattgtttcattaatgcttttgaaacagtaaatgaa
gcaataattaattttagaaaaaatttatatgcaatcaatggaaatataaaagatctatat
ttgtatttgattagatgtgatgaaaattttttcagtaaacatataactagatgtacatat
ccttctgcacttattgaaaaccaagttatatcaaataacaaaaatgaaacaaataagtta
atttttgcatttagtgattatggtcaaaaatttactaataaatttgaatggttcacaact
aaaaaaattagtagtaaccaagtgttttctgcttcgcatattaaattaaattttaaaaag
aattcaaatactaaaaataaaaatgactttacaataatcccggaaattgaagatactatc
tttagaaatcctaattatttagatttgaatactcaagcaaatttaagggcttttatatat
cctgaatatttagcttcaaaaaaaattgaatttaaaaatgaagcttattattttgaacac
aatgacaaaatatcttcatggatgaatgttcaaaataaatcatttgatagtaacaatatt
attaaacacagaaaagataaaccaattgttaaacaaattactttgtttgataaaaacaat
aaaaaaaagattattaaagttaacttttacaaagaagaattaagttcacttttttatgat
gtgtttatggaagaacaaagatgtttaaactatggttataaacaaccaaaaccctttgaa
ctattatttacttatgaaaaattccaagataaaagtgtttacttaaatgcaagtactact
aaaaaaaggggaagaatattttttgtttcaaaaacaaaaaataaagaagatattaataaa
atttattttgataaatcaggtagatttatttttgacttcaataaagaatctgtgcctttt
gcaatcacactaacaaattatgataaatctaaagatattgctgaatttgatatgttgcca
gcttgtgagaataaccctaatcaaaattttcacttggagcacgctcattcaagtcacttc
tatttaaaaccaagtaataaaatattccagcatttggaattagcaattaagcataataat
aattcttttgttttcttaaaccctaaaaagaaatatagttttgcatatgatttaattaac
cttaacttatctaagtattctaaaaatacacaaaactttatttatggaatgaatgtacat
gttccagaattaattaacattaatttatcatggatgtggaaatatcaatattttaaacct
aatatttttcttacatataatgacgaaaaaaatgatgtcaaaatagaacaagcaagatca
aatgaaaacaaaagtgatttatctaacttattatattgtttaaatactttaagtatttta
atggttgattacaataaccactataaacttaatactgatatttttgcaatgaagaataat
gcaaaagatgggaaaccatatagatggttagaatggcagaaagttaatttatcaaaatat
ccaaataaaaataatatgtgggttttgaaaaaatgtagatatgacaaagataatttttat
tggattatcagttttttcaataatgattatttatgggtgcaacaaaaaggtgaaaattgg
ggttttttctttttagcaaataaaaaaagtaagcctttaaaatcatcttcaatatttttt
cttaatgaaaatcatattaagtaa

Amino acid sequence of M. penetrans toxin
(SEQ ID NO: 79)
MINNDIKSSWLPEYYSLLENKPPKFLYRVDYKSPEEVFQKGFSINSQNEF
SFFDYFFNIYSCTSTEKEDCFINAFETVNEAIINFRKNLYAINGNIKDLY
LYLIRCDENFFSKHITRCTYPSALIENQVISNNKNETNKLIFAFSDYGQK
FTNKFEWFTTKKISSNQVFSASHIKLNFKKNSNTKNKNDFTIIPEIEDTI
FRNPNYLDLNTQANLRAFIYPEYLASKKIEFKNEAYYFEHNDKISSWMNV
QNKSFDSNNIIKHRKDKPIVKQITLFDKNNKKKIIKVNFYKEELSSLFYD
VFMEEQRCLNYGYKQPKPFELLFTYEKFQDKSVYLNASTTKKRGRIFFVS
KTKNKEDINKIYFDKSGRFIFDFNKESVPFAITLTNYDKSKDIAEFDMLP
ACENNPNQNFHLEHAHSSHFYLKPSNKIFQHLELAIKHNNNSFVFLNPKK
KYSFAYDLINLNLSKYSKNTQNFIYGMNVHVPELININLSWMWKYQYFKP
NIFLTYNDEKNDVKIEQARSNENKSDLSNLLYCLNTLSILMVDYNNHYKL
NTDIFAMKNNAKDGKPYRWLEWQKVNLSKYPNKNNMWVLKKCRYDKDNFY
MIISFFNNDYLWVQQKGENWGFFFLANKKSKPLKSSSIFFLNENHIK

Places where TGA encodes tryptophan are indicated below. Tryptophans 10, 247, 491, 493, 572, 587 and 620 (corresponding to a to g transition at nt 30, 741, 1473, 1479, 1716, 1761 and 1860) are encoded by TGA and they were changed to TGG to express recombinant M. penetrans toxin in E. coli.

Some points that distinguish between M. penetrans toxin and CARDS TX

M. penetrans toxin is similar to CARDS toxin only at two specific regions and shares 27% homology (647 vs 591 aa).

Differs in the ART conserved sequence (absence of STS).

Does auto ADP-ribosylation.

Differs in ADP ribosylation pattern from MPN372.

Needs NH₄Cl for vacuolization.

### BRIEF DESCRIPTION OF THE DRAWINGS

FIG. 1 shows an immunoblot that demonstrates both production of the CARDS toxin and anti-CARDS antibodies in three patients during infection with Mycoplasma pneumoniae.

FIG. 2 shows ADP-ribosylation of G-like proteins in HEp-2 cells following incubation with CARDS protein. Lane 1: HEp-2 cells in medium alone followed by preparation of cell free extract and addition of CARDS protein. Lane 2: HEp-2 cells pretreated with CARDS protein, followed by preparation of cell free extract and addition of CARDS protein. The marked reduction in ADP-ribosylation of specific proteins in the CARDS protein-pretreated cells is indicated by arrows. Also, ADP-ribosylation of other Hep-2 cell proteins is diminished (lane 2).

FIG. 3 shows an ELISA and an immunoblot employing rD1 as antigen that demonstrates production of anti-CARDS antibodies in sequential serum samples of two patients infected with Mycoplasma pneumoniae.

FIGS. 4A-B. Vacuolating effect of CARDS toxin on monolayers of CHO cells and HeLa cells. A. CHO cells: 10 μg/ml; panels start top, left to right; control non-intoxicated cells; then CARDS toxin-treated cells at 16 hours, 24 hours, 32 hours and 40 hours after exposure. B. HeLa cells: 10 μg/ml, panels start top, left to right; control non-intoxicated cells; then CARDS toxin-treated cells at 16 hours, 36 hours, 54 hours and 72 hours after exposure.

FIG. 5. CARDS toxin delays time to death when mice are challenged with a high lethal dose of Yersinia pestis. Mice were treated with 50 μg of CARDS toxin intranasally (IN) or 50 μg of CARDS toxin heated to 60° C. for 30 minutes (HK TOX). Four days later, animals were challenged with 40,000 CFU of Y. pestis IN. There was a delayed time to death in 50% of the toxin-treated animals at day 2. All animals died at day 3. Time is on the X axis, % survival is on the Y axis and treatment is on the Z axis.

FIG. 6. CARDS toxin delays time to death when mice in a dose dependent manner when are challenged with low lethal dose of Yersinia pestis. Mice were treated with the indicated amount of CARDS toxin IN. Four days later, animals were challenged with 800 CFU of Y. pestis. There was a delayed time to death in animals treated with 1 or 10 μg of toxin. The surviving animals appeared clinically ill and then recovered and remained alive. Time is on the X axis, % survival is on the Y axis and treatment is on the Z axis.

FIG. 7. CARDS TX treatment 2-4 days prioro to infection with lethal dose of Y. pestis provides protection. Mice were treated with 1 μg of CARDS TX IN at the indicated time prior to infection. Mice pretreated for two days were 20% protected but 3-4 days pretreatment provided 60% protection. Time is on the X axis, % survival on the Y axis and treatment on the Z axis.

FIG. 8. Growth of Y. pestis strain KIM 5 was evaluated in the presence of varying concentrations of CARDS TX. The bacteria were grown overnight in heart infusion broth with 0.2% xylose at 37° C. The culture was diluted to approximately 1000 cfu/ml and then the indicated amount of toxin or control treatment was added. Cultures were then grown an additional 1.5 hours at 37° C. and the actual number of colony forming units per ml was determined. Results are the average of duplicate experiments.

### SUMMARY OF THE INVENTION

In particular embodiments, the present invention provides Mycoplasma pneumoniae toxin (CARDS toxin) from subjects infected with Mycoplasma pneumoniae. In particular, the present invention provides a polypeptide comprising, consisting essentially of, and/or consisting of the amino acid sequence of SEQ ID NO:2 (S1 isolate), a polypeptide comprising, consisting essentially of, and/or consisting of the amino acid sequence of SEQ ID NO:3 (JL isolate), a polypeptide comprising, consisting essentially of, and/or consisting of the amino acid sequence of SEQ ID NO:4 (RJL1 isolate), a polypeptide comprising, consisting essentially of, and/or consisting of the amino acid sequence of SEQ ID NO:5 (L2 isolate), a polypeptide comprising, consisting essentially of, and/or consisting of the amino acid sequence of SEQ ID NO:1 (reference sequence), and/or a polypeptide comprising, consisting essentially of, and/or consisting of the amino acid sequence of SEQ ID NO:6 (composite sequence), either individually or in any combination.

In additional embodiments, of this invention, a Mycoplasma penetrans toxin is provided. Thus, a polypeptide of this invention as described herein includes Mycoplasma pneumoniae CARDS toxin and Mycoplasma penetrans toxin.

The present invention further provides biologically active fragments of the polypeptides of this invention, as well as antibodies that specifically bind the polypeptides and/or fragments of the polypeptides of this invention.

Further provided are nucleotide sequences that encode the polypeptides and fragments of this invention. In particular, the present invention provides an isolated nucleic acid comprising, consisting essentially of, and/or consisting of the nucleotide sequence of SEQ ID NO:8 (S1 isolate), an isolated nucleic acid comprising, consisting essentially of, and/or consisting of the nucleotide sequence of SEQ ID NO:10 (JL isolate), an isolated nucleic acid comprising, consisting essentially of, and/or consisting of the nucleotide sequence of SEQ ID NO:11 (RJL1 isolate), an isolated nucleic acid comprising, consisting essentially of, and/or consisting of the nucleotides sequence of SEQ ID NO:9 (L2 isolate), an isolated nucleic acid comprising, consisting essentially of, and/or consisting of the nucleotides sequence of SEQ ID NO:7 (reference sequence), and/or an isolated nucleic acid comprising, consisting essentially of, and/or consisting of the nucleotide sequence of SEQ ID NO:76 (composite sequence), either individually or in any combination.

Additionally provided is a nucleic acid comprising, consisting essentially of, and/or consisting of a nucleotide sequence that encodes an amino acid sequence comprising, consisting essentially of, and/or consisting of the amino acid sequence or a biologically active fragment of the amino acid sequence of SEQ ID NO:2 (S1 isolate), a nucleic acid comprising, consisting essentially of, and/or consisting of a nucleotide sequence that encodes an amino acid sequence comprising, consisting essentially of, and/or consisting of the amino acid sequence or a biologically active fragment of the amino acid sequence of SEQ ID NO:3 (JL isolate), a nucleic acid comprising, consisting essentially of, and/or consisting of a nucleotide sequence that encodes an amino acid sequence comprising, consisting essentially of, and/or consisting of the amino acid sequence or a biologically active fragment of the amino acid sequence of SEQ ID NO:4 (RJL1 isolate), a nucleic acid comprising, consisting essentially of, and/or consisting of a nucleotide sequence that encodes an amino acid comprising, consisting essentially of, and/or consisting of the amino acid sequence or a biologically active fragment of an amino acid sequence of SEQ ID NO:5 (L2) isolate, a nucleic acid comprising, consisting essentially of, and/or consisting of a nucleotide sequence that encodes an amino acid comprising, consisting essentially of, and/or consisting of the amino acid sequence or a biologically active fragment of an amino acid sequence of SEQ ID NO:1 (reference sequence) isolate, and/or a nucleic acid comprising, consisting essentially of, and/or consisting of a nucleotide sequence encoding an amino acid sequence comprising, consisting essentially of, and/or consisting of the amino acid sequence or a biologically active fragment of the amino acid sequence of SEQ ID NO:6 (composite sequence). Further provided herein is a nucleic acid that is the complement of each and any of the nucleic acids of this invention.

Also provided herein are probes and primers for the detection and/or amplification of the nucleic acids of this invention, including

| | |
|---|---|
| TTTTTACATATGCCAAATCCTGTT, | (SEQ ID NO: 12; Primer 1) |
| CGTTAAAGGATCCTCGCTAAAAGCGATC, | (SEQ ID NO: 13; Primer 2) |
| CTAGCCAAGCACTACGGACATTAGC, | (SEQ ID NO: 14; Primer 3) |
| CGTAGTGCTTGGCTAGTAGATGCTGTT, | (SEQ ID NO: 15; Primer 4) |
| CCTGGTGTTGGCAACCATGGTTG, | (SEQ ID NO: 16; Primer 5) |
| GATCAACCATGGTTGCCAACACC, | (SEQ ID NO: 17; Primer 6) |
| AAGGTGGACTCCAATCAGGGCACG, | (SEQ ID NO: 18; Primer 7) |
| CGTGCCCTGATTGGAGTCCACCTT, | (SEQ ID NO: 19; Primer 8) |
| GCGGTGTCATTTTCCACTTTTGG, | (SEQ ID NO: 20; Primer 9) |
| CCAAAAGTGGAAAATGACACCGC, | (SEQ ID NO: 21; Primer 10) |
| GGTATTCCAGAACAAGCCATTT, | (SEQ ID NO: 22; Primer 11) |
| GCTTGTTCTGGAATACCAAGAGTG, | (SEQ ID NO: 23; Primer 12) |
| ATAACCCCTATACCAGCCTAG, | (SEQ ID NO: 24; Primer 13) |
| GCTGGTATTGGAGGGGTTATTACTATACCCCACAATTAAGTGGTTGGTCTTA | (SEQ ID NO: 25; Primer 14) |
| TCAGATG, | |
| CCATTCTACCCAATCCCAGCTGTA, | (SEQ ID NO: 26; Primer 15) |
| and | |
| TACAGCTGGGATTGGGTAGAATGG. | (SEQ ID NO: 27; Primer 16) |

Additionally provided in this invention are methods of diagnosing infection by M. pneumoniae and/or M. penetrans in a subject comprising contacting a biological sample from the subject with a polypeptide or antibody of this invention under conditions whereby an antigen/antibody complex can form; and detecting formation of an antigen/antibody complex, thereby diagnosing infection by M. pneumoniae and/or M. penetrans in the subject.

Methods are also provided herein for diagnosing infection by M. pneumoniae and/or M. penetrans in a subject comprising contacting a biological sample from the subject with a nucleic acid of this invention under conditions whereby hybridization of nucleic acid molecules can occur; and detecting hybridization, thereby diagnosing infection by M. pneumoniae and/or M. penetrans in the subject.

In additional embodiments, the present invention provides a method of detecting the presence of the CARDS toxin of this invention in a sample (e.g., a biological sample from a subject or a food or water sample or other sample that could contain CARDS toxin) and/or a subject and/or diagnosing infection by M. pneumoniae in a subject, comprising contacting the sample with surfactant protein A (SP-A) under conditions whereby a toxin/SP-A complex can form; and detecting formation of the toxin/SP-A complex, thereby detecting the presence of CARDS toxin in a sample and/or diagnosing infection by M. pneumoniae in a subject.

Furthermore, the present invention provides methods of eliciting an immune response in a subject, comprising administering to the subject an effective amount of a polypeptide and/or biologically active fragment of a polypeptide of this invention and/or by administering to a subject an effective amount of a nucleic acid comprising a nucleotide sequence encoding a polypeptide and/or biologically active fragment of a polypeptide of this invention.

The present invention additionally provides methods of providing passive immunity to a subject, comprising administering to the subject an effective amount of an antibody of this invention.

In further embodiments, the present invention provides methods of treating and/or preventing infection by M. pneumoniae and/or M. penetrans in a subject, comprising administering to the subject an effective amount of a polypeptide of this invention and/or an effective amount of a biologically active fragment of a polypeptide of this invention and/or an effective amount of a nucleic acid comprising a nucleotide sequence encoding a polypeptide of this invention and/or an effective amount of a nucleic acid comprising a nucleotide sequence encoding a biologically active fragment of a polypeptide of this invention. Also provided are methods of treating and/or preventing infection by M. pneumoniae and/or M. penetrans in a subject, comprising administering to the subject an effective amount of an antibody of this invention.

In yet further embodiments, the present invention provides methods of identifying substances having the ability to inhibit or enhance various activities of the polypeptides and/or biologically active fragments of this invention, including but not limited to, binding activity, translocating activity, immunogenic activity, ADP-ribosylating activity, cytopathology inducing activity and/or toxin activity. These methods are carried out by contacting the polypeptides and/or biologically active fragments of this invention and/or the nucleic acids of this invention, with the substance to be tested for inhibitory or enhancing activity, under conditions whereby the inhibition or enhancement of activity can be detected, as described herein.

Various other objectives and advantages of the present invention will become apparent from the following detailed description.

### DETAILED DESCRIPTION OF THE INVENTION

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of virus (e.g., titer), dose (e.g., an amount of a non-viral vector), time, temperature, and the like, is meant to encompass variations of ±20%, ±10%, ±5%, ±1%, ±0.5%, or even ±0.1% of the specified amount.

The present invention is based on the discovery of polypeptides of Mycoplasma pneumoniae and M. penetrans having the respective amino acid sequence described herein and encoded by the nucleic acids described herein and the identification of activities of these polypeptides and various fragments or “domains” of these polypeptides.

Characterization of these polypeptides and fragments indicates that one newly identified protein of this invention is a toxin of Mycoplasma pneumoniae and it is referred to herein as community acquired respiratory distress syndrome (CARDS) toxin. The CARDS toxin is a cell-associated toxin. Thus, the present invention provides an isolated polypeptide comprising, consisting essentially of, and/or consisting of the amino acid sequence of SEQ ID NO:2 (S1 isolate), an isolated polypeptide comprising, consisting essentially of, and/or consisting of the amino acid sequence of SEQ ID NO:3 (JL isolate), an isolated polypeptide comprising, consisting essentially of, and/or consisting of the amino acid sequence of SEQ ID NO:4 (RJL1 isolate), an isolated polypeptide comprising, consisting essentially of, and/or consisting of the amino acid sequence of SEQ ID NO:5 (L2 isolate), an isolated polypeptide comprising, consisting essentially of, and/or consisting of the amino acid sequence of SEQ ID NO:1 (reference sequence), and/or an isolated polypeptide comprising, consisting essentially of, and/or consisting of the amino acid sequence of SEQ ID NO:6 (composite sequence), either individually or in any combination.

The present invention further provides biologically active fragments of the polypeptides of this invention, as well as antibodies that specifically bind the polypeptides and/or fragments of the polypeptides of this invention.

Further provided are nucleotide sequences that encode the polypeptides and fragments of this invention. In particular, the present invention provides an isolated nucleic acid comprising, consisting essentially of, and/or consisting of the nucleotide sequence of SEQ ID NO:8 (S1 isolate), an isolated nucleic acid comprising, consisting essentially of, and/or consisting of the nucleotide sequence of SEQ ID NO:10 (JL isolate), an isolated nucleic acid comprising, consisting essentially of, and/or consisting of the nucleotide sequence of SEQ ID NO:11 (RJL1 isolate), an isolated nucleic acid comprising, consisting essentially of, and/or consisting of the nucleotides sequence of SEQ ID NO:9 (L2 isolate), an isolated nucleic acid comprising, consisting essentially of, and/or consisting of the nucleotides sequence of SEQ ID NO:7 (reference sequence), and/or an isolated nucleic acid comprising, consisting essentially of, and/or consisting of the nucleotide sequence of SEQ ID NO:76 (composite sequence), either individually or in any combination.

Additionally provided is a nucleic acid comprising, consisting essentially of, and/or consisting of a nucleotide sequence that encodes an amino acid sequence comprising, consisting essentially of, and/or consisting of the amino acid sequence or a biologically active fragment of the amino acid sequence of SEQ ID NO:2 (S1 isolate), a nucleic acid comprising, consisting essentially of, and/or consisting of a nucleotide sequence that encodes an amino acid sequence comprising, consisting essentially of, and/or consisting of the amino acid sequence or a biologically active fragment of the amino acid sequence of SEQ ID NO:3 (JL isolate), a nucleic acid comprising, consisting essentially of, and/or consisting of a nucleotide sequence that encodes an amino acid sequence comprising, consisting essentially of, and/or consisting of the amino acid sequence or a biologically active fragment of the amino acid sequence of SEQ ID NO:4 (RJL1 isolate), a nucleic acid comprising, consisting essentially of, and/or consisting of a nucleotides sequence that encodes an amino acid comprising, consisting essentially of, and/or consisting of the amino acid sequence or a biologically active fragment of an amino acid sequence of SEQ ID NO:5 (L2 isolate), and/or a nucleic acid comprising, consisting essentially of, a nucleic acid comprising, consisting essentially of, and/or consisting of a nucleotides sequence that encodes an amino acid comprising, consisting essentially of, and/or consisting of the amino acid sequence or a biologically active fragment of an amino acid sequence of SEQ ID NO:1 (reference sequence), and/or a nucleic acid comprising, consisting essentially of, and/or consisting of a nucleotide sequence encoding an amino acid sequence comprising, consisting essentially of, and/or consisting of the amino acid sequence or a biologically active fragment of the amino acid sequence of SEQ ID NO:6 (composite sequence). Further provided herein is a nucleic acid that is the complement of each and any of the nucleic acids of this invention.

Also provided herein are probes and primers for the detection of the nucleic acids of this invention, including TTTTTACATATGCCAAATCCTGTT (SEQ ID NO:12; Primer 1), CGTTAAAGGATCCTCGCTAAAAGCGATC (SEQ ID NO:13; Primer 2), CTAGCCAAGCACTACGGACATTAGC (SEQ ID NO:14; Primer 3), CGTAGTGCTTGGCTAGTAGATGCTGTT (SEQ ID NO:15; Primer 4), CCTGGTGTTGGCAACCATGGTTG (SEQ ID NO:16; Primer 5), GATCAACCATGGTTGCCAACACC (SEQ ID NO:17; Primer 6), AAGGTGGACTCCAATCAGGGCACG (SEQ ID NO:18; Primer 7), CGTGCCCTGATTGGAGTCCACCTT (SEQ ID NO:19; Primer 8), GCGGTGTCATTTTCCACTTTTGG (SEQ ID NO:20; Primer 9), CCAAAAGTGGAAAATGACACCGC (SEQ ID NO:21; Primer 10), GGTATTCCAGAACAAGCCATTT (SEQ ID NO:22; Primer 11), GCTTGTTCTGGAATACCAAGAGTG (SEQ ID NO:23; Primer 12), ATAACCCCTATACCAGCCTAG (SEQ ID NO:24; Primer 13), GCTGGTATTGGAGGGGTTATTACTATACCCCACAATTAAGTGGTTGGTCTTA TCAGATG (SEQ ID NO:25; Primer 14), CCATTCTACCCAATCCCAGCTGTA (SEQ ID NO:26; Primer 15), and TACAGCTGGGATTGGGTAGAATGG (SEQ ID NO:27; Primer 16), alone and/or in any combination. The present invention further provides as additional embodiments without limitation, other oligonucleotides listed in this application and in the Sequence Listing attached hereto.

In additional embodiments, of this invention, a Mycoplasma penetrans toxin is provided. Thus, a polypeptide of this invention as described herein include Mycoplasma pneumoniae CARDS toxin and Mycoplasma penetrans toxin as well as any other Mycoplasma toxin defined by having the three amino acid motifs as set forth in Table 3.

Thus, in other embodiments, the present invention provides a composition comprising an isolated polypeptide comprising, consisting essentially of and/or consisting of the amino acid sequence of SEQ ID NO:79 (M. penetrans toxin) or a biologically active fragment thereof and a pharmaceutically acceptable carrier. Also provided herein is a composition comprising, consisting essentially of and/or consisting of an isolated nucleic acid comprising the nucleotide sequence of SEQ ID NO:77 (M. penetrans coding sequence) and a pharmaceutically acceptable carrier. Further provided herein is an isolated nucleic acid comprising, consisting essentially of and/or consisting of the nucleotide sequence of SEQ ID NO:78 (UGG modified coding sequence of M. penetrans toxin). This latter nucleic acid can also be present in a composition comprising a pharmaceutically acceptable carrier. In other additional embodiments, the present invention provides a composition comprising, consisting essentially of and/or consisting of an antibody that specifically binds a polypeptide comprising the amino acid sequence of SEQ ID NO:79 or an antigenic fragment thereof in a pharmaceutically acceptable carrier.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461, 463 (CCPA 1976) (emphasis in the original); see also MPEP § 2111.03. Thus, the term “consisting essentially of” when used in a claim of this invention is not intended to be interpreted to be equivalent to “comprising.”

Furthermore, the present invention provides a method of diagnosing infection by Mycoplasma penetrans in a subject, comprising contacting a biological sample from the subject with a polypeptide having the amino acid sequence of SEQ ID NO:79 (M. penetrans toxin) or an antigenic fragment thereof under conditions whereby an antigen/antibody complex can form and detecting formation of an antigen/antibody complex, thereby diagnosing infection by Mycoplasma penetrans in the subject.

Additionally provided herein is a method of diagnosing infection by Mycoplasma penetrans in a subject comprising contacting a biological sample from the subject with an antibody that specifically binds a polypeptide having the amino acid sequence of SEQ ID NO:79 under conditions whereby an antigen/antibody complex can form and detecting formation of an antigen/antibody complex, thereby diagnosing infection by Mycoplasma penetrans in the subject.

Also, the present invention provides a method of diagnosing infection by Mycoplasma penetrans in a subject, comprising contacting a biological sample from the subject with a nucleic acid comprising the nucleotide sequence of SEQ ID NO:77 (M. penetrans WT coding sequence) and/or a nucleic acid comprising the nucleotide sequence of SEQ ID NO:78 (M. penetrans coding sequence with UGG modifications) under conditions whereby hybridization of nucleic acid molecules can occur to form a hybridization complex and detecting the hybridization complex, thereby diagnosing infection by Mycoplasma penetrans in the subject.

In further aspects of this invention, a kit is provided, for detecting M. penetrans in a sample and/or for diagnosing an infection by Mycoplasma penetrans in a subject, comprising a polypeptide comprising the amino acid sequence of SEQ ID NO:79 or an antigenic fragment thereof, an antibody that specifically binds a polypeptide comprising the amino acid sequence of SEQ ID NO:79, a nucleic acid comprising the nucleotide sequence of SEQ ID NO:77 (WT), a nucleic acid comprising the nucleotide sequence of SEQ ID NO:78 (UGG modified) and any combination thereof.

Also provided herein is a method of detecting an M. penetrans toxin or an antigenic fragment thereof in a sample, comprising: a) contacting the sample with an antibody that specifically binds a polypeptide comprising the amino acid sequence of SEQ ID NO:79 under conditions whereby an antigen/antibody complex can form; and b) detecting formation of the antigen/antibody complex, thereby detecting M. penetrans toxin or an antigenic fragment in the sample.

A method is further provided of detecting an antibody to M. penetrans toxin in a sample, comprising: a) contacting the sample with a polypeptide comprising the amino acid sequence of SEQ ID NO:79 or an antigenic fragment thereof under conditions whereby an antigen/antibody complex can form; and b) detecting formation of the antigen/antibody complex, thereby detecting an antibody to M. penetrans toxin in the sample.

In addition, the present invention provides a method of detecting a nucleic acid comprising a nucleotide sequence encoding M. penetrans toxin in a sample, comprising: a) contacting the sample with an oligonucleotide comprising a nucleotide sequence that is complementary to the nucleotide sequence of SEQ ID NO:77 or SEQ ID NO:78, under conditions whereby nucleic acid hybridization can occur; and b) detecting nucleic acid hybridization, thereby detecting nucleic acid comprising the nucleotide sequence encoding M. penetrans toxin in the sample.

In further embodiments, the present invention provides a method of eliciting an immune response in a subject, comprising administering to the subject an effective amount of a polypeptide comprising the amino acid sequence of SEQ ID NO:79 or an immunogenic fragment thereof.

Also provided is a method of eliciting an immune response in a subject, comprising administering to the subject an effective amount of a nucleic acid comprising the nucleotide sequence of SEQ ID NO:77 (WT) and/or a nucleic acid comprising the nucleotide sequence of SEQ ID NO:78 (UGG modified).

The present invention further provides a method of providing passive immunity to a subject, comprising administering to the subject an effective amount of an antibody that specifically binds a polypeptide comprising the amino acid sequence of SEQ ID NO:79.

In addition, the present invention provides a method of treating or preventing infection by Mycoplasma penetrans in a subject, comprising administering to the subject an effective amount of a polypeptide comprising the amino acid of SEQ ID NO:79 or an immunogenic fragment thereof.

A method of treating or preventing infection by Mycoplasma penetrans in a subject, comprising administering to the subject an effective amount of a nucleic acid comprising the nucleotide sequence of SEQ ID NO:77 (WT) and/or a nucleic acid comprising the nucleotide sequence of SEQ ID NO:78 (UGG modified).

Further provided is a method of treating or preventing infection by Mycoplasma penetrans in a subject, comprising administering to the subject an effective amount of an antibody that specifically binds a polypeptide comprising the amino acid sequence of SEQ ID NO:79.

Further embodiments of this invention are based on the unexpected discovery of an immunomodulating activity of the CARDS toxin and the M. penetrans toxin. Thus, the present invention also provides a method of treating or preventing a detrimental immune response in a subject, comprising administering to the subject an effective amount of CARDS toxin and/or M. penetrans toxin or an active fragment thereof.

Further provided is a method of enhancing an immunomodulating effect of a substance, comprising combining the substance with CARDS toxin or an active fragment thereof.

Also provided herein is a method of treating and/or preventing Yersinia pestis infection in a subject, comprising administering to the subject an effective amount of CARDS toxin and/or M. penetrans toxin or an active fragment thereof and/or a nucleotide sequence encoding CARDS toxin and/or M. penetrans toxin or an active fragment thereof. The methods of this invention can be used to treat an infection caused by any pathogen that elicits a Th1 immune response, as described herein.

In particular embodiments of this invention, the CARDS TX described herein can elicit a predictable pattern of chemokines and cytokines that essentially holds ‘in check’ specific pathogens (e.g., Y. pestis). During this initial protective period, the CARDS TX-dependent response recruits cells of the immune system. The result of this pattern of host stimulation results in (1) activation of innate immunity (increases in macrophages and dendritic cells) which kill a variety of pathogens, including intracellular microorganisms; (2) creation of a favorable environment for the host to neutralize, kill, inhibit, or reverse subsequent disease progression by stimulating and attracting CD4 Th1 cells, which in turn increase CD8 cytotoxic T cell activity; and (3) stimulation of other protective pathways, including the family of interferons, which has been shown to result in powerful antiviral and antifungal activities.

In addition, the present invention provides a method of treating or preventing a detrimental immune response in a subject, comprising administering to the subject an effective amount of a nucleic acid encoding CARDS toxin or an active fragment thereof.

A detrimental immune response as described herein can be, for example, a detrimental immune response produced by a pathogenic agent that acts to inhibit the host's natural immune response in order to establish infection and/or cause disease. Nonlimiting examples of such pathogenic agents include Yersinia pestis, human immunodeficiency virus (HIV), Mycobacterium tuberculosis, Francisella tularensis and any other pathogens now known or later identified to elicit a Th1 immune response. Examples of a detrimental immune response include but are not limited to allergic reaction or allergy, immune-mediated inflammation of the organs (e.g., heart; central nervous system; kidney; liver), pathogen-induced immunopathology, autoimmune diseases and disorders (e.g., diabetes, SLE, MS), etc.

Also provided herein is a method of treating or preventing a detrimental immune response in a subject wherein the detrimental immune response is caused by CARDS toxin activity, comprising administering to the subject an effective amount of a substance that inhibits CARDS toxin activity. A substance that inhibits CARDS toxin activity, can be, but is not limited to a ligand (e.g., an antibody or antibody fragment) that specifically binds a CARDS toxin or active fragment thereof and/or a nucleic acid that inhibits transcription or translation of nucleic acid encoding a CARDS toxin or active fragment thereof (e.g., an antisense nucleic acid that binds a coding sequence of the A35R protein, an interfering RNA that inhibits or suppresses transcription and/or translation of the A35R protein, a ribozyme, etc.) Furthermore, small molecules and other compounds and substances that inhibit the activity of CARDS toxin could be used in the methods of this invention.

In other embodiments, the present invention provides a method of enhancing an immunomodulating effect of a substance, comprising combining the substance with a nucleic acid encoding CARDS toxin or an active fragment thereof. Such a substance that has an immunomodulating effect can be but is not limited to steroids, immunosuppressive drugs, interferons, corticosteroids, azathioprine, cyclophosphamide, prednisone, methotrexate, rituximab, etc., as well as any other immunomodulating agent now known or later identified.

An enhancement of an immunomodulating effect of a substance would be identified by comparison of an immunomodulating effect of the substance in a subject with and without the presence of the CARDS toxin or active fragment thereof or nucleic acid encoding the CARDS toxin or active fragment thereof. As used herein, an “immunomodulating effect” is any action or activity of a cell or tissue of the immune system. Such an immunomodulating effect can be positive or negative, an enhancement or an inhibition, an increase or a decrease in a response, activity and/or reaction. Ways to detect and/or measure an immunomodulating effect are known in the art and include standard protocols such as those used to detect and/or measure antibody production or activity, T lymphocyte proliferation or activity, cytotoxicity and/or cytokine production or activity.

In additional embodiments, the present invention provides a method of treating or preventing an autoimmune disorder in a subject, comprising administering to the subject an effective amount of CARDS toxin or an active fragment thereof and/or a nucleic acid encoding a CARDS toxin or active fragment thereof.

Nonlimiting examples of autoimmune disorders that can be treated and/or prevented by the methods of this invention include acute and chronic arthritis (e.g., rheumatoid arthritis or RA), multiple sclerosis (MS), diabetes (e.g., insulin dependent diabetes mellitus or IDDM), systemic lupus erythematosus (SLE), myasthenia gravis, Crohns' disease, regional enteritis, vasculitis, ulcerative colitis, Sjogren's syndrome, ankylosing spondylitis, polymyositis and any other autoimmune disorder now known or later identified. The methods of the present invention can further be employed to treat allergies, allergic reactions, and any other disease or disorder associated with an aberrant and/or undesirable immune response or reaction.

Additionally provided is a method of reducing the likelihood of transplant rejection (or increasing the likelihood of successful transplantation) in a transplant recipient, comprising administering to the transplant recipient an effective amount of CARDS toxin or an active fragment thereof and/or administering to the transplant recipient an effective amount of a nucleic acid encoding a CARDS toxin or active fragment thereof. The reduction in the likelihood of transplant rejection or increase in the likelihood of successful transplantation is in comparison to the likelihood of transplant rejection or likelihood of successful transplantation in a transplant recipient that did not receive a CARDS toxin or active fragment thereof or a nucleic acid encoding a CARDS toxin or active fragment thereof, as such likelihoods would be know and/or determined according to art-known standards. Furthermore, the protein, active fragment and/or nucleic acid of these methods can be administered to the transplant recipient at any time relative to the transplantation (i.e., before, after and/or simultaneously, in any combination.

Also provided herein is a method of modulating (e.g., enhancing or inhibiting) an immune response in a subject, comprising administering to the subject an effective amount of CARDS toxin or an active fragment thereof and/or a nucleic acid encoding CARDS toxin or an active fragment thereof.

In methods provided herein for enhancing an immune response, such an enhancement is identified by comparison with an immune response in a subject that did not receive the protein, active fragment and/or nucleic acid of this invention. In methods provided herein for inhibiting an immune response, such an inhibition is identified by comparison with an immune response in a subject that did not receive the protein, active fragment and/or nucleic acid of this invention. Such comparative studies can be carried out according to well known protocols in the art for detecting and/or measuring any number of immune responses. Nonlimiting examples of an immune response that can be enhanced by the methods of this invention include antibody response (e.g., protective antibody response; neutralizing antibody response), cytotoxic T cell response, T helper response, interleukin-2 (IL-2) production; and vaccine efficacy.

Additional embodiments of this invention provide a method of treating and/or preventing an obstructive airway disorder in a subject, comprising administering to the subject an effective amount of an inhibitor of CARDS toxin activity and/or an inhibitor of M. penetrans toxin activity.

An obstructive airway disease that can be treated and/or prevented according to the present invention can include, but is not limited to, asthma, allergy, pneumonia, tracheobronchitis, pharyngitis, croup, chronic obstructive pulmonary disease (COPD), sarcoidosis, interstitial pneumonia, interstitial pulmonary fibrosis, hypersensitivity pneumonitis, antiphospholipid syndrome, bronchiolitis and other chronic lung and allergic diseases and disorders, in any combination. In particular embodiments, the obstructive airway disorder is asthma.

The present invention also contemplates the treatment or prevention of allergies and/or allergic reaction, caused by various allergens, which can include, but are not limited to, environmental allergens such as dust mite allergens; plant allergens such as pollen, including ragweed pollen; insect allergens such as bee and ant venom; and animal allergens such as cat dander, dog dander and animal saliva allergens.

An inhibitor of CARDS toxin activity can be an antibody or other ligand that specifically binds CARDS toxin and inhibits CARDS toxin activity. An inhibitor of CARDS toxin activity can also be a nucleic acid that inhibits the expression of a gene encoding the CARDS toxin and/or that inhibits transcription of CARDS toxin mRNA and/or that inhibits translation of the CARDS toxin from mRNA. (e.g., interfering RNA, antisense nucleic acid sequences, ribozymes, etc.).

An inhibitor of M. penetrans toxin activity can be an antibody or other ligand that specifically binds M. penetrans toxin and inhibits M. penetrans toxin activity. An inhibitor of M. penetrans toxin activity can also be a nucleic acid that inhibits the expression of a gene encoding the M. penetrans toxin and/or that inhibits transcription of M. penetrans toxin mRNA and/or that inhibits translation of the M. penetrans toxin from mRNA (e.g., interfering RNA, antisense nucleic acid sequences, ribozymes, etc.). As used herein, the term “inhibit” or “inhibits” includes complete or partial reduction in an activity of the toxin described herein, as determined according to protocols known in the art and as described herein.

“Isolated” as used herein means the nucleic acid or polypeptide of this invention is sufficiently free of contaminants or cell components with which nucleic acids or polypeptides normally occur. “Isolated” does not mean that the preparation is technically pure (homogeneous), but it is sufficiently pure to provide the nucleic acid or polypeptide in a form in which it can be used therapeutically.

“Epitope” or “antigenic epitope” or “antigenic peptide” or “antigenic fragment” or “immunogenic fragment” as used herein means a specific amino acid sequence of limited length which, when present in the proper conformation, provides a reactive site for an antibody or T cell receptor. The identification of epitopes on antigens can be carried out by immunology protocols that are well known in the art.

As used herein, the term “polypeptide” or “protein” is used to describe a chain of amino acids that correspond to those encoded by a nucleic acid. A polypeptide of this invention can be a peptide, which usually describes a chain of amino acids of from two to about 30 amino acids. The term polypeptide as used herein also describes a chain of amino acids having more than 30 amino acids and can be a fragment or domain of a protein or a full length protein. Furthermore, as used herein, the term polypeptide can refer to a linear chain of amino acids or it can refer to a chain of amino acids that has been processed and folded into a functional protein. It is understood, however, that 30 is an arbitrary number with regard to distinguishing peptides and polypeptides and the terms can be used interchangeably for a chain of amino acids. The polypeptides of the present invention are obtained by isolation and purification of the polypeptides from cells where they are produced naturally, by enzymatic (e.g., proteolytic) cleavage, and/or recombinantly by expression of nucleic acid encoding the polypeptides or fragments of this invention. The polypeptides and/or fragments of this invention can also be obtained by chemical synthesis or other known protocols for producing polypeptides and fragments.

The amino acid sequences disclosed herein are presented in the amino to carboxy direction, from left to right. Nucleotide sequences are presented herein by single strand only, in the 5′ to 3′ direction, from left to right. However, it is intended that the nucleic acids of this invention can be either single or double stranded (i.e., including the complementary nucleic acid). A nucleic acid of this invention can be the complement of a nucleic acid described herein.

A “biologically active fragment” includes a polypeptide of this invention that comprises a sufficient number of amino acids to have one or more of the biological activities of the polypeptides of this invention. Such biological activities can include, but are not limited to, in any combination, binding activity, translocating activity, immunogenic activity, ADP-ribosylating activity, and/or cytopathology inducing activity, as well as any other activity now known or later identified for the polypeptides and/or fragments of this invention. A fragment of a polypeptide of this invention can be produced by methods well known and routine in the art. Fragments of this invention can be produced, for example, by enzymatic or other cleavage of naturally occurring peptides or polypeptides or by synthetic protocols that are well known. Such fragments can be tested for one or more of the biological activities of this invention according to the methods described herein, which are routine methods for testing activities of polypeptides, and/or according to any art-known and routine methods for identifying such activities. Such production and testing to identify biologically active fragments of the polypeptides described herein would be well within the scope of one of ordinary skill in the art and would be routine.

Fragments of the polypeptides of this invention are preferably at least about ten amino acids in length and retain one or more of the biological activities and/or the immunological activities of the CARDS toxin. Examples of the fragments of this invention include, but are not intended to be limited to, the following fragments identified by the amino acid number as shown in the Sequence Listing for each of the isolates of SEQ ID NO:2 (S1 isolate), SEQ ID NO:3 (JL isolate), SEQ ID NO:4 (RJL1 isolate), SEQ ID NO:5 (L2 isolate), SEQ ID NO:6 (composite sequence) and SEQ ID NO:1 (reference sequence): Amino acids 1-10, 10-20, 20-30, 30-40, 40-50, 50-60, 60-70, 70-80, 80-90, 90-100, 110-120, 120-130, 130-140, 140-150, 150-160, 160-170, 170-180, 180-190, 190-200, 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420, 420-430, 430-440, 440-450, 450-460, 460-470, 470-480, 480-490, 490-500, 500-510, 510-520, 520-530, 530-540, 540-550, 550-560, 560-570, 570-580, 580-591, 1-25, 1-50, 1-67, 1-75, 1-100, 1-125, 1-135, 1-145, 1-150, 1-160, 1-170, 1-180, 1-190, 1-200, 1-250, 1-300, 1-350, 1-400, 1-450, 1-500, 68-180, 183-123, 500-591, 450-591, 400-591, 350-591, 300-591, 250-591, 200-591, 150-591, 100-591, 50-591, 50-100, 100-200, 200-300, 300-400, 400-500, 500-591, 550-591.

It is understood that this list is exemplary only and that a fragment of this invention can be any amino acid sequence containing any combination of contiguous amino acids that are numbered in the Sequence Listing as amino acids 1 through 591 even if that combination is not specifically recited as an example herein. It is also understood that these fragments can be combined in any order or amount. For example, fragment 1-10 can be combined with fragment 10-20 to produce a fragment of amino acids 1-20. Also fragments can be present in multiple numbers and in any combination in a fragment of this invention. Thus, for example, fragment 1-150 can be combined with a second fragment 1-150 and/or combined with fragment 400-500 to produce a fragment of this invention. Other exemplary fragments of this invention include the domains of the CARDS toxin described herein [e.g., domain 1 (N terminal 249 amino acids), domain 2 (256 amino acids) and domain 3 (247 amino acids at carboxy terminus)].

The terms “homology,” “identity” and “complementarity” as used herein refer to a degree of similarity between two or more sequences. There may be partial homology or complete homology (i.e., identity). A partially complementary sequence that at least partially inhibits an identical sequence from hybridizing to a target nucleic acid is referred to as “substantially homologous.” The inhibition of hybridization of the completely complementary sequence to the target sequence can be examined using a hybridization assay (Southern or Northern blot, solution hybridization and the like) under conditions of low stringency. A substantially homologous sequence or hybridization probe will compete for and inhibit the binding of a completely homologous sequence to the target sequence under conditions of low stringency, as this term is known in the art. This is not to say that conditions of low stringency are such that non-specific binding is permitted; low stringency conditions require that the binding of two sequences to one another be a specific (i.e., selective) interaction. The absence of non-specific binding can be tested by the use of a second target sequence that lacks even a partial degree of complementarity (e.g., less than about 30% identity). In the absence of non-specific binding, the probe will not hybridize to the second non-complementary target sequence.

The term “hybridization” as used herein refers to any process by which a first strand of nucleic acid binds with a second strand of nucleic acid through base pairing. Nucleic acids encoding the polypeptides and/or fragments of this invention can be detected by DNA-DNA or DNA-RNA hybridization or amplification using probes, primers and/or fragments of polynucleotides encoding the polypeptides and/or fragments of this invention and/or designed to detect and/or amplify the nucleic acids of this invention.

The term “hybridization complex” as used herein refers to a complex formed between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀t or R₀t analysis) or between one nucleic acid sequence present in solution and another nucleic acid sequence immobilized on a solid support (e.g., paper, membranes, filters, chips, pins or glass slides, or any other appropriate substrate to which cells and/or nucleic acids have been fixed).

The term “nucleotide sequence” refers to a heteropolymer of nucleotides or the sequence of these nucleotides. The terms “nucleic acid,” “oligonucleotide” and “polynucleotide” are also used interchangeably herein to refer to a heteropolymer of nucleotides. Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene. Nucleic acids of this invention can comprise a nucleotide sequence that can be identical in sequence to the sequence which is naturally occurring or, due to the well-characterized degeneracy of the nucleic acid code, can include alternative codons which encode the same amino acid as that which is found in the naturally occurring sequence. Furthermore, nucleic acids of this invention can comprise nucleotide sequences that can include codons which represent conservative substitutions of amino acids as are well known in the art, such that the biological activity of the resulting polypeptide and/or fragment is retained. For example, modifications of the toxins of this invention leading to specific amino acid substitutions (e.g., changing the catalytic glutamate at position 132 to alanine; or changing the serine-threonine-serine motif at amino acids 49-51; or changing the arginine at position 10 to alanine) can alter the toxin's ADP ribosylation, vacuolating and immunomodulatory activities and make the toxins better candidates as vaccine and/or immunomodulatory molecules. Other amino acid substitutions as would be known to one of skill in the art can alter other structural and/or functional properties of the CARDS TX and M. penetrans toxin to improve their therapeutic capabilities. In certain embodiments, the predictive adjuvant properties of CARDS TX and/or the M. penetrans toxin of this invention can be used as an adjuvant and/or in combination with other antigens (viral, bacterial, protozoal, fungal) to enhance the immunogenic properties of such antigens, e.g., for vaccine purposes. This would be most beneficial when the other antigen (i.e., not toxin) is poorly antigenic.

Because of the immunomodulatory Th1-type properties of the toxin of this invention, the toxin should be able to force the shifting of a Th2 response that is elicited by many environmental stresses, including specific infectious agents and allergens, to a Th1 response that would benefit the well-being of the host (human and animal) by reducing inflammation and other clinical signs and symptoms.

Furthermore, treatment of drug-resistant bacteria such as, for example, Mycobacterium tuberculosis and Staphylococcus species with the toxin of this invention, with and without additional anti-microbials, could markedly improve patient outcomes.

In addition, immunocompromised individuals could greatly benefit from a toxin of this invention and/or derivatives, including peptide fragments and amino acid substituted regions, because of the Th1-like type response elicited which would be helpful in dealing with many pathological states.

The term “probe” or “primer” includes naturally occurring or recombinant or chemically synthesized single- and/or double-stranded nucleic acids. They can be labeled for detection by nick translation, Klenow fill-in reaction, PCR or other methods well known in the art. Probes and primers of the present invention, their preparation and/or labeling are described in Sambrook et al. 1989. Molecular Cloning: A Laboratory Manual, Cold Spring Harbor Laboratory, NY and Ausubel et al. 1989. Current Protocols in Molecular Biology, John Wiley & Sons, New York N.Y., both of which are incorporated herein by reference in their entirety for these teachings.

The term “stringent” as used here refers to hybridization conditions that are commonly understood in the art to define the conditions of the hybridization procedure. Stringency conditions can be low, high or medium, as those terms are commonly know in the art and well recognized by one of ordinary skill. In various embodiments, stringent conditions can include, for example, highly stringent (i.e., high stringency) conditions (e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C.), and/or moderately stringent (i.e., medium stringency) conditions (e.g., washing in 0.2×SSC/0.1% SDS at 42° C.).

“Amplification” as used herein includes the production of multiple copies of a nucleic acid molecule and is generally carried out using polymerase chain reaction (PCR) and/or other amplification technologies as are well known in the art (Dieffenbach and Dveksler. 1995. PCR Primer, a Laboratory Manual, Cold Spring Harbor Press, Plainview, N.Y.).

As used herein, the term “antibody” includes intact immunoglobulin molecules as well as fragments thereof, such as Fab, F(ab′)2, and Fc, which are capable of binding the epitopic determinant of an antigen (i.e., antigenic determinant). Antibodies that bind the polypeptides of this invention are prepared using intact polypeptides or fragments containing small peptides of interest as the immunizing antigen. The polypeptide or fragment used to immunize an animal can be derived from enzymatic cleavage, recombinant expression, isolation from biological materials, synthesis, etc., and can be conjugated to a carrier protein, if desired. Commonly used carriers that are chemically coupled to peptides and proteins for the production of antibody include, but are not limited to, bovine serum albumin, thyroglobulin and keyhole limpet hemocyanin. The coupled peptide or protein is then used to immunize the animal (e.g., a mouse, rat, or rabbit). The polypeptide or peptide antigens can also be administered with an adjuvant, as described herein and as otherwise known in the art.

The term “antibody” or “antibodies” as used herein refers to all types of immunoglobulins, including IgG, IgM, IgA, IgD, and IgE. The antibody can be monoclonal or polyclonal and can be of any species of origin, including, for example, mouse, rat, rabbit, horse, goat, sheep or human, or can be a chimeric or humanized antibody. See, e.g., Walker et al., Molec. Immunol. 26:403-11 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980. The antibody can further be a single chain antibody or bispecific antibody.

Antibody fragments included within the scope of the present invention include, for example, Fab, F(ab′)2, and Fc fragments, and the corresponding fragments obtained from antibodies other than IgG. Such fragments can be produced by known techniques. For example, F(ab′)2 fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (Huse et al., (1989) Science 254:1275-1281).

Monoclonal antibodies can be produced in a hybridoma cell line according to the technique of Kohler and Milstein, (1975) Nature 265:495-97. For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in bacterial cell such as E. coli by recombinant techniques known to those skilled in the art. See, e.g., W. Huse, (1989) Science 246:1275-81.

Antibodies can also be obtained by phage display techniques known in the art or by immunizing a heterologous host with a cell containing an epitope of interest.

The term “sample” as used herein is used in its broadest sense. A biological sample suspected of containing a polypeptide, fragment, antibody and/or nucleic acid of this invention can be any biological fluid, an extract from a cell, an extracellular matrix isolated from a cell, a cell (in solution or bound to a solid support), a tissue, a tissue print, and the like.

“Effective amount” refers to an amount of a compound or composition of this invention that is sufficient to produce a desired effect, which can be a therapeutic effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science And Practice of Pharmacy (20th ed. 2000)).

A “pharmaceutically acceptable” component such as a salt, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include, without limitation, any of the standard pharmaceutical carriers such as phosphate buffered saline solutions, water, emulsions such as oil/water emulsion, microemulsions and various types of wetting agents. In particular, it is intended that a pharmaceutically acceptable carrier be a sterile carrier that is formulated for administration to or delivery into a subject of this invention.

“Treat,” “treating” or “treatment” refers to any type of action that imparts a modulating effect, which, for example, can be a beneficial effect, to a subject afflicted with a disorder, disease or illness, including improvement in the condition of the subject (e.g., in one or more symptoms), delay in the progression of the condition, prevention or delay of the onset of the disorder, and/or change in clinical parameters, disease or illness, etc., as would be well known in the art.

As used herein, “modulate,” “modulates” or “modulation” refers to enhancement (e.g., an increase) or inhibition (e.g., diminished, reduced or suppressed) of the specified activity.

The term “enhancement,” “enhance,” “enhances,” or “enhancing” refers to an increase in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or an increase in the specified parameter of at least about 5%, 10%, 25%, 35%, 40%, 50%, 60%, 75%, 80%, 90%, 95%, 97%, 98%, 99% or 100%.

The term “inhibit,” “diminish,” “reduce” or “suppress” refers to a decrease in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or a decrease or reduction in the specified parameter of at least about 5%, 10%, 25%, 35%, 40%, 50%, 60%, 75%, 80%, 90%, 95%, 97%, 98%, 99% or 100%. In particular embodiments, the inhibition or reduction results in little or essentially no detectible activity (at most, an insignificant amount, e.g., less than about 10% or about 5%).

An “immunomodulatory molecule” of this invention can be, but is not limited to an immunostimulatory cytokine that can be, but is not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules.

Additional examples of an immunomodulatory molecule of this invention include the adjuvants of this invention, including, for example, SYNTEX adjuvant fommulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153 (the entire contents of which are incorporated herein by reference), or a less reactogenic composition where the QS21 is quenched with cholesterol as disclosed in PCT publication number WO 96/33739 (the entire contents of which are incorporated herein by reference). A particularly potent adjuvant formulation involving QS21 3D-MPL & tocopherol in an oil in water emulsion is described in PCT publication number WO 95/17210 (the entire contents of which are incorporated herein by reference). In addition, the nucleic acid of this invention can include an adjuvant by comprising a nucleotide sequence encoding a A35R protein or active fragment thereof of this invention and a nucleotide sequence that provides an adjuvant function, such as CpG sequences. Such CpG sequences, or motifs, are well known in the art.

A subject of this invention includes any animal susceptible to infection by Mycoplasma pneumoniae and/or M penetrans. Such a subject can be a mammal and in particular embodiments, is a human. As used herein, a “subject” or “subject in need thereof” is a subject known to be, or suspected of being, infected with Mycoplasma pneumoniae and/or M penetrans. A subject of this invention can also include a subject not previously known or suspected to be infected by Mycoplasma pneumoniae and/or M. penetrans or in need of treatment for Mycoplasma pneumoniae and/or M. penetrans infection. For example, a subject of this invention can be administered the compositions of this invention even if it is not known or suspected that the subject is infected with Mycoplasma pneumoniae and/or M. penetrans (e.g., prophylactically). A subject of this invention is also a subject known or believed to be at risk of infection by Mycoplasma pneumoniae and/or M penetrans.

In certain embodiments, the fragments and/or polypeptides of this invention can be fused with a “carrier” protein or peptide to produce a fusion protein. For example, the carrier protein or peptide can be fused to a polypeptide and/or fragment of this invention to increase the stability thereof (e.g., decrease the turnover rate) in the cell and/or subject. Exemplary carrier proteins include, but are not limited to, glutathione-S-transferase or maltose-binding protein. The carrier protein or peptide can alternatively be a reporter protein. For example, the fusion protein can comprise a polypeptide and/or fragment of this invention and a reporter protein or peptide (e.g., Green Fluorescent Protein, β-glucoronidase, β-galactosidase, luciferase, and the like) for easy detection of transformed cells and transgene expression. As a further alternative, the fusion protein attached to the polypeptides and/or fragments and a carrier protein or peptide can be targeted to a subcellular compartment of interest, i.e., to affect the co-localization of the polypeptide and/or fragment. Any suitable carrier protein as is well known in the art can be used to produce a fusion protein of this invention.

The polypeptides and/or fragments of the present invention can 1) be used in assays to determine the biological activity of other proteins or peptides; 2) be included in a panel of multiple proteins for high-throughput screening; 3) be used to raise antibodies or to elicit an immune response; 4) be used as a reagent (including the labeled reagent) in assays designed to quantitatively determine levels of the protein (or its binding partner or receptor) in biological fluids; and 5) be used as markers for tissues in which the corresponding protein is preferentially expressed (either constitutively or at a particular stage of tissue differentiation or development or in a disease state). Any or all of these research utilities are capable of being developed into reagent grade or kit format for commercialization as research products. Methods for performing the uses listed above are well known to those skilled in the art. References disclosing such methods include Molecular Cloning: A Laboratory Manual, 2d ed., Cold Spring Harbor Laboratory Press, Sambrook et al., eds. (1989) and Methods in Enzymology: Guide to Molecular Cloning Techniques, Academic Press, Berger and Kimmel eds. (1987).

A variety of protocols for detecting the presence of and/or measuring the amount of polypeptides, fragments and/or peptides in a sample, using either polyclonal or monoclonal antibodies specific for the polypeptide, fragment and/or peptide are known in the art. Examples of such protocols include, but are not limited to, enzyme immunoassays (EIA), agglutination assays, immunoblots (Western blot; dot/slot blot, etc.), radioimmunoassays (RIA), immunodiffusion assays, chemiluminescence assays, antibody library screens, expression arrays, enzyme-linked immunosorbent assays (ELISA), radioimmunoassays (RIA), immunoprecipitation, Western blotting, competitive binding assays, immunofluorescence, immunohistochemical staining precipitation/flocculation assays and fluorescence-activated cell sorting (FACS). These and other assays are described, among other places, in Hampton et al. (Serological Methods, a Laboratory Manual, APS Press, St Paul, Minn. (1990)) and Maddox et al. (J. Exp. Med. 158:1211-1216 (1993)).

Furthermore, a number of assays for detection and/or amplification of nucleic acid sequences are well known in the art. Additionally, a wide variety of labeling and conjugation techniques are known in the art that are used in various nucleic acid detection and amplification assays. Methods for producing labeled hybridization probes and/or PCR or other ligation primers for detecting and/or amplifying nucleic acid sequences can include, for example, oligolabeling, nick translation and end-labeling, as well as other well known methods. Alternatively, nucleic acid sequences encoding the polypeptides of this invention, and/or any functional fragment thereof, can be cloned into a plasmid or vector for detection and amplification. Such plasmids and vectors are well known in the art and are commercially available. It is also contemplated that the methods of this invention can be conducted using a variety of commercially-available kits (e.g., Pharmacia & Upjohn; Promega; U.S. Biochemical Corp.). Suitable reporter molecules or labels, which can be used for ease of detection, include, for example, radionuclides, enzymes, fluorescence agents, chemiluminescence agents and chromogenic agents, as well as substrates, cofactors, inhibitors, magnetic particles and the like as are well known in the art.

The present invention further includes isolated polypeptides, peptides, proteins, fragments, domains and/or nucleic acid molecules that are substantially equivalent to those described for this invention. As used herein, “substantially equivalent” can refer both to nucleic acid and amino acid sequences, for example a mutant sequence, that varies from a reference sequence by one or more substitutions, deletions, or additions, the net effect of which does not result in an undesirable adverse functional dissimilarity between reference and subject sequences. In some embodiments, this invention can include substantially equivalent sequences that have an adverse functional dissimilarity. For purposes of the present invention, sequences having equivalent biological activity and equivalent expression characteristics are considered substantially equivalent.

The invention further provides homologs, as well as methods of obtaining homologs, of the polypeptides and/or fragments of this invention from other strains of Mycoplasma and/or other organisms. As used herein, an amino acid sequence or protein is defined as a homolog of a polypeptide or fragment of the present invention if it shares significant homology to one of the polypeptides and/or fragments of the present invention. Significant homology means at least 75%, 80%, 85%, 90%, 95%, 98% and/or 100% homology with another amino acid sequence. Specifically, by using the nucleic acids disclosed herein as a probe or as primers, and techniques such as PCR amplification and colony/plaque hybridization, one skilled in the art can identify homologs of the polypeptides and/or fragments of this invention in Mycoplasma and/or other organisms.

The present invention also provides an antibody that specifically binds the polypeptides and/or biologically active fragments of this invention, as well as a method of making an antibody specific for a polypeptide and/or fragment of this invention comprising: a) immunizing an animal with a polypeptide and/or fragment of this invention under conditions whereby the animal produces antibodies that specifically bind the polypeptide and/or fragment of this invention; and b) removing biological materials comprising the antibodies from the animal. Also provided herein is an antibody produced by the methods set forth herein.

Antibodies of this invention can be generated using methods that are well known in the art. Such antibodies and immunoglobulin molecules of this invention can include, but are not limited to, polyclonal antibodies, monoclonal antibodies, chimeric antibodies, humanized antibodies, single chain antibodies (e.g., scFv), Fab fragments, and fragments produced by a Fab expression library.

In general, techniques for preparing polyclonal and monoclonal antibodies as well as hybridomas capable of producing a desired antibody are well known in the art. Any animal known to produce antibodies can be immunized with a polypeptide, fragment and/or antigenic epitope of this invention. Methods for immunization of animals to produce antibodies are well known in the art. For example, such methods can include subcutaneous or interperitoneal injection of the polypeptide, fragment and/or antigenic epitope of this invention.

The polypeptide, fragment or antigenic epitope that is used as an immunogen can be modified or administered in an adjuvant in order to increase antigenicity. Methods of increasing the antigenicity of a protein or peptide are well known in the art and include, but are not limited to, coupling the antigen with a heterologous protein (such as globulin or β-galactosidase) or through the inclusion of an adjuvant during immunization.

For example, for the production of antibodies, various hosts including goats, rabbits, rats, mice, humans, and others, can be immunized by injection with the polypeptides and/or fragments of this invention, with or without a carrier protein. Additionally, various adjuvants may be used to increase the immunological response. Such adjuvants include, but are not limited to, Freund's complete and incomplete adjuvants, mineral gels such as aluminum hydroxide, and surface-active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable.

Polypeptides, peptides and/or fragments of this invention used as antigens to produce the antibodies of this invention can have an amino acid sequence consisting of at least five amino acids and in certain embodiments, at least ten amino acids. In one embodiment, the antigen is identical to a portion of the amino acid sequence of the natural protein, and it can contain the entire amino acid sequence of a small, naturally-occurring molecule. Short stretches of the polypeptides and/or fragments of this invention can be fused with all or a fragment of another protein that acts as a carrier protein (e.g., keyhole limpet hemocyanin) and antibodies can be produced against the chimeric polypeptide or peptide.

Monoclonal antibodies to the polypeptides and/or fragments of this invention are prepared using any technique, which provides for the production of antibody molecules by continuous cell lines in culture. These include, but are not limited to, the hybridoma technique, the human B-cell hybridoma technique, and the EBV-hybridoma technique (Kohler et al. 1975. Nature 256:495-497; Kozbor et al. 1985. J. Immunol. Methods 81:31-42; Cote et al. 1983. Proc. Natl. Acad. Sci. 80:2026-2030; Cole et al. 1984. Mol. Cell. Biol. 62:109-120).

For example, to produce monoclonal antibodies, spleen cells from the immunized animal are removed, fused with myeloma cells, and cultured in selective medium to become monoclonal antibody-producing hybridoma cells, according to techniques routine in the art. Any one of a number of methods well known in the art can be used to identify the hybridoma cell, which produces an antibody with the desired characteristics. These include screening the hybridomas by ELISA assay, Western blot analysis, or radioimmunoassay. Hybridomas secreting the desired antibodies are cloned and the class and subclass are identified using standard procedures known in the art.

For polyclonal antibodies, antibody-containing serum is isolated from the immunized animal and is screened for the presence of antibodies with the desired specificity using any of the well known procedures as described herein.

The present invention further provides antibodies of this invention in detectably labeled form. Antibodies can be detectably labeled through the use of radioisotopes, affinity labels (such as biotin, avidin, etc.), enzymatic labels (such as horseradish peroxidase, alkaline phosphatase, etc.) fluorescence labels (such as FITC or rhodamine, etc.), paramagnetic atoms, gold beads, etc. Such labeling procedures are well-known in the art. The labeled antibodies of the present invention can be used for in vitro, in vivo, and in situ assays to identify a polypeptide and/or fragment of this invention in a sample.

In some embodiments, the present invention further provides the above-described antibodies immobilized on a solid support (e.g., beads, plates, slides or wells formed from materials such as latex or polystyrene). Examples of such solid supports include plastics such as polycarbonate, complex carbohydrates such as agarose and sepharose, acrylic resins and such as polyacrylamide and latex beads. Techniques for coupling antibodies to such solid supports are well known in the art (Weir et al., Handbook of Experimental Immunology 4th Ed., Blackwell Scientific Publications, Oxford, England, Chapter 10 (1986)). Antibodies can likewise be conjugated to detectable groups such as radiolabels (e.g., ³⁵S, ¹²⁵I, ¹³¹I), enzyme labels (e.g., horseradish peroxidase, alkaline phosphatase), and fluorescence labels (e.g., fluorescein) in accordance with known techniques. Determination of the formation of an antibody/antigen complex in the methods of this invention can be by detection of, for example, precipitation, agglutination, flocculation, radioactivity, color development or change, fluorescence, luminescence, etc., as is well know in the art.

In addition, techniques developed for the production of chimeric antibodies or humanized antibodies by splicing mouse antibody genes to human antibody genes to obtain a molecule with appropriate antigen specificity and biological activity can be used (Morrison et al. 1984. Proc. Natl. Acad. Sci. 81:6851-6855; Neuberger et al. 1984. Nature 312:604-608; Takeda et al. 1985. Nature 314:452-454). Alternatively, techniques described for the production of single chain antibodies can be adapted, using methods known in the art, to produce single chain antibodies specific for the polypeptides and fragments of this invention. Antibodies with related specificity, but of distinct idiotypic composition, can be generated by chain shuffling from random combinatorial immunoglobulin libraries (Burton 1991. Proc. Natl. Acad. Sci. 88:11120-3).

Antibody fragments that specifically bind the polypeptides and/or fragments of this invention can also be generated. For example, such fragments include, but are not limited to, the F(ab′)₂ fragments that can be produced by pepsin digestion of the antibody molecule and the Fab fragments that can be generated by reducing the disulfide bridges of the F(ab′)₂ fragments. Alternatively, Fab expression libraries may be constructed to allow rapid and easy identification of monoclonal Fab fragments with the desired specificity (Huse et al. 1989. Science 254:1275-1281).

Various immunoassays can be used for screening to identify antibodies having the desired specificity for the proteins and peptides of this invention. Numerous protocols for competitive binding or immunoradiometric assays using either polyclonal or monoclonal antibodies with established specificity are well known in the art. Such immunoassays typically involve the measurement of complex formation between an antigen and its specific antibody (e.g., antigen/antibody complex formation). For example, a two-site, monoclonal-based immunoassay utilizing monoclonal antibodies reactive to two non-interfering epitopes on the proteins or peptides of this invention can be used, as well as a competitive binding assay.

It is further contemplated that the present invention provides kits for detection of the polypeptides and/or fragments of this invention in a sample. In one embodiment, the kit can comprise one or more antibodies of this invention, along with suitable buffers, wash solutions and/or other reagents for the detection of antibody/antigen complex formation. In an alternative embodiment, a kit of this invention can comprise a polypeptide, an antigenic peptide of the polypeptide of this invention, a fragment of this invention and/or an antigenic peptide of a fragment of this invention, along with suitable buffers, wash solutions and/or other reagents for the detection of antibody/antigen complex formation.

The present invention further provides a kit for the detection of nucleic acid encoding the polypeptides and/or fragments of this invention. For example, in one embodiment, the kit can comprise one or more nucleic acids of this invention, along with suitable buffers, wash solutions and/or other reagents for the detection of hybridization complex formation.

It would be well understood by one of ordinary skill in the art that the kits of this invention can comprise one or more containers and/or receptacles to hold the reagents (e.g., antibodies, antigens, nucleic acids) of the kit, along with appropriate buffers and/or wash solutions and directions for using the kit, as would be well known in the art. Such kits can further comprise adjuvants and/or other immunostimulatory or immunomodulating agents, as are well known in the art.

In further embodiments, the nucleic acids encoding the polypeptides and/or fragments of this invention can be part of a recombinant nucleic acid construct comprising any combination of restriction sites and/or functional elements as are well known in the art which facilitate molecular cloning and other recombinant DNA manipulations. Thus, the present invention further provides a recombinant nucleic acid construct comprising a nucleic acid encoding a polypeptide and/or biologically active fragment of this invention.

The present invention further provides a vector comprising a nucleic acid encoding a polypeptide and/or fragment of this invention. The vector can be an expression vector which contains all of the genetic components required for expression of the nucleic acid in cells into which the vector has been introduced, as are well known in the art. The expression vector can be a commercial expression vector or it can be constructed in the laboratory according to standard molecular biology protocols. The expression vector can comprise viral nucleic acid including, but not limited to, vaccinia virus, adenovirus, retrovirus and/or adeno-associated virus nucleic acid. The nucleic acid or vector of this invention can also be in a liposome or a delivery vehicle, which can be taken up by a cell via receptor-mediated or other type of endocytosis.

The nucleic acid of this invention can be in a cell, which can be a cell expressing the nucleic acid whereby a polypeptide and/or biologically active fragment of this invention is produced in the cell. In addition, the vector of this invention can be in a cell, which can be a cell expressing the nucleic acid of the vector whereby a polypeptide and/or biologically active fragment of this invention is produced in the cell. It is also contemplated that the nucleic acids and/or vectors of this invention can be present in a host animal (e.g., a transgenic animal), which expresses the nucleic acids of this invention and produces the polypeptides and/or fragments of this invention.

The nucleic acid encoding the polypeptide and/or fragment of this invention can be any nucleic acid that functionally encodes the polypeptides and/or fragments of this invention. To functionally encode the polypeptides and/or fragments (i.e., allow the nucleic acids to be expressed), the nucleic acid of this invention can include, for example, expression control sequences, such as an origin of replication, a promoter, an enhancer and necessary information processing sites, such as ribosome binding sites, RNA splice sites, polyadenylation sites and transcriptional terminator sequences.

Preferred expression control sequences are promoters derived from metallothionine genes, actin genes, immunoglobulin genes, CMV, SV40, adenovirus, bovine papilloma virus, etc. A nucleic acid encoding a selected polypeptide and/or fragment can readily be determined based upon the genetic code for the amino acid sequence of the selected polypeptide and/or fragment and many nucleic acids will encode any selected polypeptide and/or fragment. Modifications in the nucleic acid sequence encoding the polypeptide and/or fragment are also contemplated. Modifications that can be useful are modifications to the sequences controlling expression of the polypeptide and/or fragment to make production of the polypeptide and/or fragment inducible or repressible as controlled by the appropriate inducer or repressor. Such methods are standard in the art. The nucleic acid of this invention can be generated by means standard in the art, such as by recombinant nucleic acid techniques and by synthetic nucleic acid synthesis or in vitro enzymatic synthesis.

In yet further embodiments, the present invention provides a D1 domain of CARDS Toxin comprising, consisting essentially of and/or consisting of the amino acid sequence of SEQ ID NO:69 and/or SEQ ID NO:75, a D2 domain of CARDS Toxin comprising, consisting essentially of, or consisting of the amino acid sequence of SEQ ID NO: 70, and/or a D3 domain of CARDS Toxin comprising, consisting essentially of, and/or consisting of the amino acid sequence of SEQ ID NO:71, in any combination.

Further provided herein is an isolated nucleic acid encoding the amino acid sequence of the domains D1, D2 and D3 of this invention. As one example, a nucleic acid encoding the domain D1 can comprise, consist of and/or consist essentially of the nucleotide sequence of SEQ ID NO:74.

Additionally provided herein are antibodies that specifically bind domain D1, D2 and/or D3 of the CARDS Toxin of this invention. The domain peptides can be used as antigens for the production of antibodies, which can be polyclonal and/or monoclonal, according to well known protocols. The domain peptides and antibodies can be used in the methods described herein for the detection of M. pneunoniae antibodies and proteins and/or for diagnosis of M. pneumoniae infection, as well as in therapeutic methods to treat M. pneumoniae infection and related diseases as described herein.

The present invention further provides a method of producing a polypeptide and/or biologically active fragment according to the methods set forth in the Examples provided herein, and as are well known in the art for polypeptide synthesis. In one embodiment, a nucleic acid encoding the polypeptides and/or fragments of this invention can be synthesized according to standard nucleic acid synthesis protocols and the nucleic acid can be expressed according to methods well known for expression of nucleic acid. The resulting polypeptide and/or fragment can then be removed from the expression system by standard isolation and purification procedures and tested for any of the various biological activities described herein according to methods as taught herein as well as methods routine in the art.

The present invention also provides a method for producing the polypeptides and/or biologically active fragments of this invention comprising producing the cells of this invention which contain the nucleic acids or vectors of this invention as exogenous nucleic acid; culturing the cells under conditions whereby the exogenous nucleic acid in the cell can be expressed and the encoded polypeptide and/or fragment can be produced; and isolating the polypeptide and/or fragment from the cell. Thus, it is contemplated that the polypeptides and/or fragments of this invention can be produced in quantity in vitro in either prokaryotic or eukaryotic expression systems as are well known in the art.

As one example, for expression in a prokaryotic system, there are numerous E. coli (Escherichia coli) expression vectors known to one of ordinary skill in the art useful for the expression of nucleic acid that encodes polypeptides. Other microbial hosts suitable for use include bacilli, such as Bacillus subtilis, and other enterobacteria, such as Salmonella, Serratia, as well as various Pseudomonas species. These prokaryotic hosts can support expression vectors that will typically contain expression control sequences compatible with the host cell (e.g., an origin of replication). In addition, any number of a variety of well-known promoters can be present, such as the lactose promoter system, a tryptophan (Trp) promoter system, a beta-lactamase promoter system, or a promoter system from phage lambda. The promoters will typically control expression, optionally with an operator sequence and have ribosome binding site sequences for example, for initiating and completing transcription and translation. If necessary, an amino terminal methionine can be provided by insertion of a Met codon 5′ and in-frame with the polypeptide. Also, the carboxy-terminal extension of the polypeptide can be removed using standard oligonucleotide mutagenesis procedures.

The nucleic acid sequences can be expressed in hosts after the sequences have been positioned to ensure the functioning of an expression control sequence. These expression vectors are typically replicable in the host organisms either as episomes or as an integral part of the host chromosomal DNA. Commonly, expression vectors can contain selection markers, e.g., tetracycline resistance or hygromycin resistance, to permit detection and/or selection of those cells transformed with the desired nucleic acid sequences.

As another example, for eukaryotic system expression, a yeast expression system can be used. There are several advantages to yeast expression systems. First, evidence exists that polypeptides produced in a yeast expression system exhibit correct disulfide pairing. Second, post-translational glycosylation is efficiently carried out by yeast expression systems. The Saccharomyces cerevisiae pre-pro-alpha-factor leader region (encoded by the MFα-1 gene) is routinely used to direct protein secretion from yeast. The leader region of pre-pro-alpha-factor contains a signal peptide and a pro-segment, which includes a recognition sequence for a yeast protease encoded by the KEX2 gene. This enzyme cleaves the precursor protein on the carboxyl side of a Lys-Arg dipeptide cleavage-signal sequence. The polypeptide coding sequence can be fused in-frame to the pre-pro-alpha-factor leader region. This construct is then put under the control of a strong transcription promoter, such as the alcohol dehydrogenase I promoter or a glycolytic promoter. The coding sequence is followed by a translation termination codon, which is followed by transcription termination signals. Alternatively, the coding sequence of interest can be fused to a second polypeptide coding sequence, such as Sj26 or β-galactosidase, used to facilitate purification of the resulting fusion polypeptide by affinity chromatography. The insertion of protease cleavage sites to separate the components of the fusion polypeptide is applicable to constructs used for expression in yeast.

Efficient post-translational glycosylation and expression of recombinant polypeptides can also be achieved in Baculovirus systems in insect cells, as are well known in the art.

In yet further embodiments, the peptides, polypeptides and/or fragments of this invention can be expressed in mammalian cells. Mammalian cells permit the expression of peptides and polypeptides in an environment that favors important post-translational modifications such as folding and cysteine pairing, addition of complex carbohydrate structures and secretion of active protein. Vectors useful for the expression of peptides and polypeptides in mammalian cells are characterized by insertion of the coding sequence between a strong (e.g., viral) promoter and a polyadenylation signal. The vectors can contain genes conferring either, e.g., gentamicin or methotrexate resistance, for use as selectable markers. For example, the coding sequence can be introduced into a Chinese hamster ovary (CHO) cell line using a methotrexate resistance-encoding vector. Presence of the vector RNA in transformed cells can be confirmed by Northern blot analysis and production of a cDNA or opposite strand RNA corresponding to the polypeptide or fragment coding sequence can be confirmed by Southern and Northern blot analysis, respectively. A number of other suitable host cell lines capable of producing exogenous polypeptides have been developed in the art and include the CHO cell lines, HeLa cells, myeloma cell lines, Jurkat cells and the like. Expression vectors for these cells can include expression control sequences, as described above.

The nucleic acids and/or vectors of this invention can be transferred into the host cell by well-known methods, which vary depending on the type of cell host. For example, calcium chloride transfection is commonly used for prokaryotic cells, whereas calcium phosphate treatment or electroporation can be used for other cell hosts.

The polypeptides, fragments, nucleic acids, vectors and cells of this invention can be present in a pharmaceutically acceptable carrier. By “pharmaceutically acceptable” is meant a material that is not biologically or otherwise undesirable, i.e., the material may be administered to an individual along with the selected polypeptide, fragment, nucleic acid, vector or cell without causing substantial deleterious biological effects or interacting in a deleterious manner with any of the other components of the composition in which it is contained.

Furthermore, any of the compositions of this invention can comprise a pharmaceutically acceptable carrier and a suitable adjuvant. As used herein, “suitable adjuvant” describes an adjuvant capable of being combined with the polypeptide and/or fragment and/or nucleic acid of this invention to further enhance an immune response without deleterious effect on the subject or the cell of the subject. A suitable adjuvant can be, but is not limited to, MONTANIDE ISA51 (Seppic, Inc., Fairfield, N.J.), SYNTEX adjuvant formulation 1 (SAF-1), composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Other suitable adjuvants are well known in the art and include QS-21, Freund's adjuvant (complete and incomplete), alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

The compositions of the present invention can also include other medicinal agents, pharmaceutical agents, carriers, diluents, immunostimulatory cytokines, etc. Actual methods of preparing such dosage forms are known, or will be apparent, to those skilled in this art.

It is contemplated that the above-described compositions of this invention can be administered to a subject or to a cell of a subject to impart a therapeutic benefit. Thus, the present invention further provides a method of producing an immune response in a subject, comprising administering to the subject or to a cell of the subject an effective amount of a polypeptide and/or biologically active fragment of this invention and/or a nucleic acid comprising a nucleotide sequence encoding a polypeptide and/or biologically active fragment of this invention. The cell of the subject can be in vivo or ex vivo and can be, but is not limited to a CD8+ T lymphocyte (e.g., a cytotoxic T lymphocyte) or an MHC I-expressing antigen presenting cell, such as a dendritic cell, a macrophage and/or a monocyte. Detection of an immune response in the subject or in the cells of the subject can be carried out according to methods standard in the art for detecting a humoral and/or cellular immune response.

Furthermore, the present invention provides a method of eliciting an immune response in a subject, comprising administering to the subject an effective amount of a polypeptide and/of fragment of this invention.

Also provided herein is a method of eliciting an immune response in a subject, comprising administering to the subject an effective amount of a nucleic acid and/or vector of this invention.

In additional embodiments, the present invention provides a method of providing passive immunity to a subject, comprising administering to the subject an effective amount of an antibody of this invention to the subject.

The compositions of this invention can also be employed as a therapeutic and/or prophylactic formulation and administered to a subject in need thereof. Thus, the present invention provides a method of treating or preventing infection or intoxication by Mycoplasma pneumoniae and/or M. penetrans in a subject, comprising administering to the subject an effective amount of a polypeptide and/or fragment of this invention, a nucleic acid and/or vector of this invention, and/or an antibody of this invention.

In addition, the present invention provides a method of treating or preventing infection or intoxication caused by Mycoplasma pneumoniae and/or M. penetrans in a subject comprising contacting an immune cell of the subject with any of the polypeptides, fragments, nucleic acids, vectors and/or antibodies of this invention. The cell can be in vivo or ex vivo and can be, for example, a CD8⁺ T cell which is contacted with the polypeptide and/or fragment of this invention in the presence of a class I MHC molecule, which can be a soluble molecule or it can be present on the surface of a cell which expresses class I MHC molecules. The cell can also be an antigen presenting cell or other class I MHC-expressing cell which can be contacted with the nucleic acids and/or vectors of this invention under conditions whereby the nucleic acid or vector is introduced into the cell by standard methods for uptake of nucleic acid and vectors. The nucleic acid encoding the polypeptide and/or fragment of this invention is then expressed and the polypeptide and/or fragment product is processed within the antigen presenting cell or other MHC I-expressing cell and presented on the cell surface as an MHC I/antigen complex. The antigen presenting cell or other class I MHC-expressing cell is then contacted with an immune cell of the subject which binds the class I MHC/antigen complex and elicits an immune response which treats or prevents Mycoplasma pneumoniae and/or M. penetrans infection in the subject.

As set forth above, it is contemplated that in the methods wherein the compositions of this invention are administered to a subject or to a cell of a subject, such methods can further comprise the step of administering a suitable adjuvant to the subject or to a cell of the subject. The adjuvant can be in the composition of this invention or the adjuvant can be in a separate composition comprising the suitable adjuvant and a pharmaceutically acceptable carrier. The adjuvant can be administered prior to, simultaneous with, or after administration of the composition containing any of the polypeptides, fragments, nucleic acids and/or vectors of this invention. For example, QS-21, similar to alum, complete Freund's adjuvant, SAF, etc., can be administered within days/weeks/hours (before or after) of administration of the composition of this invention. The effectiveness of an adjuvant can be determined by measuring the immune response directed against the polypeptide and/or fragment of this invention with and without the adjuvant, using standard procedures, as described in the Examples herein and as are well known in the art.

As set forth above, the subject of this invention can be any subject in need of the immune response of this invention and/or in need of treatment for or prevention from Mycoplasma pneumoniae and/or M. penetrans infection, as well as any subject in whom it is desirable to induce an immune response to Mycoplasma pneumoniae and/or M penetrans. Symptoms of Mycoplasma pneumoniae infection can include tracheobronchitis and pneumonia with extrapulmonary pathologies, such as neurologic, cardiac, gastrointestinal, dermatologic, renal and joint complications. A range of serological (elevated IgM and IgG seroconversion) assays and PCR detection can be used for diagnosing M. pneumoniae infection. Appropriate treatment can lead to resolution of respiratory symptoms such as decreased fever and cough, complete recovery of respiratory function including normal lung radiogram, and normal levels of tissue enzymes and CSF analysis. Also, decreased levels of M. pneumoniae and/or M. penetrans cells, antigens and nucleic acids in blood, sputum, bronchial lavage should identify an effective treatment.

Symptoms of M. penetrans infection can include AIDS progression (multiple tissue involvement and clinical manifestations), urethritis, acute and chronic kidney infections, respiratory disease, antiphospholipid syndrome, etc. as would be known in the art. Effective treatment would result in, for example, improved immune function, reduction or elimination of tissue-associated pathologies, reduction of fever and reduction of tissue inflammation.

Common sources of infection can include infected individuals coughing, sneezing and transmitting aerosols containing M. pneumoniae. The transmission rate is very high, which is why M. pneumoniae is such a common cause of community acquired pneumonia. Highest targets of infection are children, especially 5-9 years old and adults between ages 25-40, although infection can occur among all ‘healthy’ individuals. Thus, a subject for whom the methods of this invention would be indicated for preventing M. pneumoniae infection can, in some embodiments, be a child or young adult.

The compositions of this invention can be administered to a cell of a subject or to a subject either in vivo or ex vivo. For administration to a cell of the subject in vivo, as well as for administration to the subject, the compositions of this invention can be administered orally, parenterally (e.g., intravenously), by intramuscular injection, by intraperitoneal injection, subcutaneous injection, transdermally, extracorporeally, topically or the like. Also, the compositions of this invention can be pulsed onto dendritic cells, which are isolated or grown from a subject's cells, according to methods well known in the art, or onto bulk peripheral blood mononuclear cells (PBMC) or various cell subfractions thereof from a subject.

The exact amount of the composition required will vary from subject to subject, depending on the species, age, weight and general condition of the subject, the particular composition used, its mode of administration and the like. Thus, it is not possible to specify an exact amount for every composition of this invention. However, effective amount can be determined by one of ordinary skill in the art using only routine experimentation given the teachings herein.

As an example, to a subject diagnosed with M. pneumoniae and/or M. penetrans infection or known to be at risk of being infected with M. pneumoniae and/or M. penetrans or in whom it is desirable to induce an immune response to Mycoplasma pneumoniae and/or M. penetrans, between about 50-1000 nM and more preferably, between about 100-500 nM of a polypeptide and/or biologically active fragment of this invention can be administered subcutaneously and can be in an adjuvant, at one to three hour/day/week intervals until an evaluation of the subject's clinical parameters indicate that the subject is not infected by M. pneumoniae and/or M. penetrans and/or the subject demonstrates the desired immunological response. Alternatively, a polypeptide and/or fragment of this invention can be pulsed onto dendritic cells at a concentration of between about 10-1100 μM and the dendritic cells can be administered to the subject intravenously at the same time intervals. The treatment can be continued or resumed if the subject's clinical parameters indicate that M. pneumoniae and/or M. penetrans infection is present and can be maintained until the infection is no longer detected by these parameters and/or until the desired immunological response is achieved.

If ex vivo methods are employed, cells or tissues can be removed and maintained outside the subject's body according to standard protocols well known in the art. The polypeptides and/or biologically active fragments of this invention can be introduced into the cells via known mechanisms for uptake of polypeptides into cells (e.g., phagocytosis, pulsing onto class I MHC-expressing cells, liposomes, etc.). The cells can then be infused (e.g., in a pharmaceutically acceptable carrier) or transplanted back into the subject per standard methods for the cell or tissue type. Standard methods are known for transplantation or infusion of various cells into a subject.

The nucleic acids and vectors of this invention can also be administered to a cell of the subject either in vivo or ex vivo. The cell can be any cell that can take up and express exogenous nucleic acid and produce the polypeptides and/or fragments of this invention. In some embodiments, the polypeptides and/or fragments of this invention can be produced by a cell that secretes them, whereby the polypeptide and/or fragment is produced and secreted and then taken up and subsequently processed by an antigen presenting cell or other class I MHC-expressing cell and presented to the immune system for induction of an immune response. In other embodiments, the nucleic acids and/or vectors of this invention can be directly introduced into an antigen presenting cell and/or other class I MHC-expressing cell in which the polypeptide and/or fragment is produced and processed directly and presented to the immune system on the cell surface.

The nucleic acids and vectors of this invention can be administered orally, intranasally, parenterally (e.g., intravenously), by intramuscular injection, by intraperitoneal injection, transdermally, extracorporeally, topically or the like. In the methods described herein which include the administration and uptake of exogenous DNA into the cells of a subject (i.e., gene transduction or transfection), the nucleic acids of the present invention can be in the form of naked DNA or the nucleic acids can be in a vector for delivering the nucleic acids to the cells for expression of the polypeptides and/or fragments of this invention. The vector can be a commercially available preparation or can be constructed in the laboratory according to methods well known in the art.

Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system, which can package a recombinant retroviral genome. The recombinant retrovirus can then be used to infect and thereby deliver to the infected cells nucleic acid encoding the polypeptide and/or fragment of this invention. The exact method of introducing the exogenous nucleic acid into mammalian cells is, of course, not limited to the use of retroviral vectors. Other techniques are widely available for this procedure including the use of adenoviral vectors, alphaviral vectors, adeno-associated viral (AAV) vectors, lentiviral vectors, pseudotyped retroviral vectors and vaccinia viral vectors, as well as any other viral vectors now known or developed in the future. Physical transduction techniques can also be used, such as liposome delivery and receptor-mediated and other endocytosis mechanisms. This invention can be used in conjunction with any of these or other commonly used gene transfer methods.

As another example, if the nucleic acid of this invention is delivered to the cells of a subject in an adenovirus vector, the dosage for administration of adenovirus to humans can range from about 10⁷ to 10⁹ plaque forming units (pfu) per injection, but can be as high as 10¹², 10¹⁵ and/or 10²⁰ pfu per injection. Ideally, a subject will receive a single injection. If additional injections are necessary, they can be repeated at daily/weekly/monthly intervals for an indefinite period and/or until the efficacy of the treatment has been established. As set forth herein, the efficacy of treatment can be determined by evaluating the symptoms and clinical parameters described herein and/or by detecting a desired immunological response.

The exact amount of the nucleic acid or vector required will vary from subject to subject, depending on the species, age, weight and general condition of the subject, the particular nucleic acid or vector used, its mode of administration and the like. Thus, it is not possible to specify an exact amount for every nucleic acid or vector. However, an appropriate amount can be determined by one of ordinary skill in the art using only routine experimentation given the teachings herein.

If ex vivo methods are employed, cells or tissues can be removed and maintained outside the body according to standard protocols well known in the art. The nucleic acids and vectors of this invention can be introduced into the cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or transplanted back into the subject per standard methods for the cell or tissue type. Standard methods are known for transplantation or infusion of various cells into a subject.

Parenteral administration of the peptides, polypeptides, nucleic acids and/or vectors of the present invention, if used, is generally characterized by injection. Injectables can be prepared in conventional forms, either as liquid solutions or suspensions, solid forms suitable for solution of suspension in liquid prior to injection, or as emulsions. As used herein, “parenteral administration” includes intradermal, intranasal, subcutaneous, intramuscular, intraperitoneal, intravenous and intratracheal routes, as well as a slow release or sustained release system such that a constant dosage is maintained. See, e.g., U.S. Pat. No. 3,610,795, which is incorporated by reference herein in its entirety.

The efficacy of treating or preventing Mycoplasma pneumoniae and/or M. penetrans infection by the methods of the present invention can be determined by detecting a clinical improvement as indicated by a change in the subject's symptoms and/or clinical parameters, as would be well known to one of skill in the art.

It is further contemplated that the compositions of the present invention can be used in diagnostic and therapeutic applications. Thus, the present invention provides a method of detecting the presence of a polypeptide and/or fragment of this invention in a sample, comprising contacting the sample with an antibody of this invention under conditions whereby an antigen/antibody complex can form and detecting formation of an antigen/antibody complex, thereby detecting the presence of a Mycoplasma pneumoniae polypeptide and/or fragment and/or a Mycoplasma penetrans toxin of fragment thereof of this invention in the sample.

Additionally, the present invention provides a method of detecting the presence of an antibody of this invention in a sample, comprising contacting the sample with a polypeptide and/or fragment of this invention under conditions whereby an antigen/antibody complex can form and detecting formation of an antigen/antibody complex, thereby detecting the presence of a Mycoplasma pneumoniae and/or M. penetrans antibody of this invention in the sample.

In additional embodiments, the present invention provides a method of detecting the presence of the CARDS toxin of this invention in a sample (e.g., a biological sample from a subject or a food or water sample or other sample that could contain CARDS toxin) and/or a subject and/or diagnosing infection by M. pneumoniae in a subject, comprising contacting the sample with surfactant protein A (SP-A) under conditions whereby a toxin/SP-A complex can form; and detecting formation of the toxin/SP-A complex, thereby detecting the presence of CARDS toxin in a sample and/or diagnosing infection by M. pneumoniae in a subject.

In the detection and diagnosis methods of this invention, it would be well understood by the ordinary artisan that any variety of protocols could be carried to detect formation of an antigen/antibody complex or a toxin/SP-A complex. For example, a secondary antigen, secondary ligand and/or secondary antibody that is detectably labeled can be employed (e.g., a “sandwich immunoassay”).

The sample of this invention can be any sample in which Mycoplasma pneumoniae CARDS toxin and/or M. penetrans toxin can be present. For example, the sample can be a body fluid, cells or tissue that can contain Mycoplasma pneumoniae toxin and/or M. penetrans toxin, including but not limited to, blood, serum, plasma, saliva, sputum, bronchoalveolar lavage, urine, semen, joint fluid, cerebrospinal fluid and cells, fluids and/or tissue from all organs to which CARDS toxin can disseminate including lung, liver, heart, brain, kidney, spleen, muscle, etc. A sample of this invention can also be a sample in which a polypeptide, nucleic acid and/or fragment of this invention can be detected, such as water, soil, effluent, food, feedstuff, air, swabs of surfaces,

Additionally, the present invention provides a method of diagnosing Mycoplasma pneumoniae and/or M. penetrans infection in a subject comprising contacting a biological sample from the subject with a polypeptide and/or fragment of this invention under conditions whereby an antigen/antibody complex can form; and detecting formation of an antigen/antibody complex, thereby diagnosing Mycoplasma pneumoniae and/or M. penetrans infection in the subject.

A method of diagnosing Mycoplasma pneumoniae and/or M. penetrans infection in a subject is further provided, comprising contacting a biological sample from the subject with an antibody of this invention under conditions whereby an antigen/antibody complex can form; and detecting formation of an antigen/antibody complex, thereby diagnosing Mycoplasma pneumoniae and/or M. penetrans infection in the subject.

In further embodiments, the present invention provides a method of diagnosing infection by Mycoplasma pneumoniae and/or M. penetrans in a subject, comprising contacting a biological sample from the subject with the nucleic acid of this invention under conditions whereby hybridization of nucleic acid molecules can occur and detecting a hybridization complex, thereby diagnosing infection by Mycoplasma pneumoniae and/or M. penetrans in the subject.

In additional embodiments, the present invention provides a method of identifying a subject infected with Mycoplasma pneumoniae and/or M. penetrans as having a poor prognosis, comprising: a) establishing a correlation between the presence of and/or an amount of a polypeptide, fragment, nucleic acid and/or antibody of this invention in a sample of test subjects infected with Mycoplasma pneumoniae and/or M. penetrans and who have or had a poor prognosis; and b) detecting in a biological sample from the subject the presence of and/or an amount of the polypeptide, fragment, nucleic acid and/or antibody of this invention correlated with a poor prognosis, thereby identifying the subject infected with Mycoplasma pneumoniae and/or M. penetrans as having a poor prognosis. For example, a correlation can be made between a level of antibodies to the CARDS toxin and/or antibodies to M. penetrans and a degree of respiratory and/or pulmonary dysfunction indicative of a poor prognosis.

The present invention also provides various screening assays that employ the polypeptides, fragments and/or nucleic acids of this invention. In particular, provided herein is a method of identifying a substance having the ability to inhibit or enhance the binding activity of a polypeptide and/or biologically active fragment of this invention comprising contacting the substance with the CARDS protein or a biologically active fragment thereof under conditions whereby binding can occur and detecting a decrease or increase in the amount of binding in the presence of the substance as compared to a control amount of binding in the absence of the substance, thereby identifying a substance having the ability to inhibit or enhance the binding activity of the CARDS toxin.

Inhibition or enhancement of binding activity can be detected by any of a variety of art-recognized methods for evaluating binding activity. As one example, the substance to be tested and the CARDS polypeptide and/or fragment can be contacted in the presence of target cells or a target substrate (e.g., surfactant protein A; SP-A) known to bind the polypeptide or fragment. The amount of binding of polypeptide or fragment to the cells or the substrate in the presence of the substance and the amount of binding of polypeptide or fragment to the cells or the substrate in the absence of the substance is determined and a decrease or increase in the amount of binding in the presence of the substance identifies the substance as having the ability to inhibit or enhance binding.

In some embodiments, binding of polypeptide and/or fragment to target cells or a target substrate can be measured by attaching a detectable moiety to the polypeptide or fragment (e.g., a fluorescence moiety, histochemically detectable moiety, radioactive moiety, etc.). The amount of detectable moiety can be measured in the presence and absence of the substance to be tested and the amounts can be compared to determine inhibition or enhancement. Binding activity can also be determined by comparing the amount of cytopathology observed in a monolayer of target cells in the presence and absence of the substance to be tested. Target cells that can be used in such a binding assay include, but are not limited to, Chinese hamster ovary (CHO) cells, Hep2 cells, human lung and kidney epithelial and fibroblast cells, and any other mammalian cells that exhibit sensitivity to CARDS toxin now known or later identified.

In addition, the present invention provides a method of identifying a substance having the ability to inhibit or enhance the translocating activity of a polypeptide and/or a biologically active fragment of this invention, comprising contacting the substance with the polypeptide of this invention and/or a biologically active fragment thereof under conditions whereby translocation activity can occur and detecting a decrease or increase in the amount of translocation activity in the presence of the substance as compared to a control amount of translocation activity in the absence of the substance, thereby identifying a substance having the ability to inhibit or enhance the translocating activity of the CARDS toxin.

Inhibition or enhancement of translocating activity can be detected by any of a variety of art-recognized methods for evaluating translocating activity. As one example, the substance to be tested and the CARDS polypeptide and/or fragment can be contacted in the presence of target cells known to translocate the CARDS toxin. The amount of translocation of polypeptide or fragment into the cells in the presence of the substance and the amount of translocation of polypeptide or fragment into the cells in the absence of the substance is determined and a decrease or increase in the amount of translocation in the presence of the substance identifies the substance as having the ability to inhibit or enhance translocation of the CARDS toxin. Translocation of polypeptide and/or fragment into target cells can be measured by attaching a detectable moiety to the polypeptide or fragment (e.g., a fluorescence moiety, histochemically detectable moiety, radioactive moiety, etc.). The amount of translocated detectable moiety can be measured in the presence and absence of the substance to be tested and the amounts can be compared to determine inhibition or enhancement of translocation. Translocation activity can also be determined by comparing the amount of cytopathology observed in a monolayer of target cells in the presence and absence of the substance to be tested. Target cells that can be used in such a translocation assay include, but are not limited to, Chinese hamster ovary (CHO) cells, etc.

Further provided is a method of identifying a substance having the ability to enhance or inhibit the immunogenic activity of the CARDS toxin of this invention and/or a biologically active fragment thereof, comprising contacting the substance with the CARDS toxin or an immunogenic fragment thereof under conditions whereby a measurable immune response can be elicited and detecting an increase or decrease in the amount of immune response in the presence of the substance, as compared to a control amount of immune response in the absence of the substance, thereby identifying a substance having the ability to enhance or inhibit immunogenic activity of the CARDS toxin. Assays to detect and measure immune responses are well known in the art and can be employed to detect either humoral or cellular immune responses.

In additional embodiments, the present invention provides a method of identifying a substance having the ability to inhibit or enhance the ADP-ribosylating activity of the CARDS toxin of this invention and/or biologically active fragments thereof, comprising contacting the substance with the CARDS toxin or biologically active fragment thereof under conditions whereby ADP ribosylation can occur and detecting a decrease or increase in the amount of ADP ribosylation in the presence of the substance as compared to a control amount of ADP ribosylation in the absence of the substance, thereby identifying a substance having the ability to inhibit or enhance the ADP ribosylating activity of the CARDS toxin.

Methods for detecting ADP ribosylating activity are well known in the art and are described, for example, in the Examples section provided herein.

Further provided is a method of identifying a substance having the ability to inhibit or enhance the cytopathology-inducing activity of the CARDS toxin of this invention and/or a biologically active fragment thereof, comprising contacting the substance with the CARDS toxin or biologically active fragment thereof under conditions whereby cytopathology (e.g., changes in cell morphology, monolayer characteristics, etc.) of target cells can be induced and detecting a decrease or increase in the amount of cytopathology in the presence of the substance, as compared to a control amount of cytopathology in the absence of the substance, thereby identifying a substance having the ability to inhibit or enhance the cytopathology-inducing activity of the CARDS toxin or biologically active fragment thereof.

Methods of detecting cytopathology of cells are well known in the art and are described, for example, in the Examples section herein.

Additionally provided is a method of identifying a substance having the ability to inhibit the binding activity of the M. penetrans toxin, comprising contacting the substance with the toxin or a biologically active fragment thereof under conditions whereby binding can occur and detecting a decrease in the amount of binding in the presence of the substance as compared to a control amount of binding in the absence of the substance, thereby identifying a substance having the ability to inhibit the binding activity of the M. penetrans toxin.

A method is also provided herein of identifying a substance having the ability to enhance the immunogenic activity of the M. penetrans toxin, comprising contacting the substance with the toxin or an immunogenic fragment thereof under conditions whereby a measurable immune response can be elicited and detecting in increase in the amount of immune response in the presence of the substance, as compared to a control amount of immune response in the absence of the substance, thereby identifying a substance having the ability to enhance immunogenic activity of the M. penetrans toxin.

Also provided herein is a method of identifying a substance having the ability to inhibit the ADP-ribosylating activity of the M. penetrans toxin, comprising contacting the substance with the toxin or biologically active fragment thereof under conditions whereby ADP ribosylation can occur and detecting a decrease in the amount of ADP ribosylation in the presence of the substance as compared to a control amount of ADP ribosylation in the absence of the substance, thereby identifying a substance having the ability to inhibit the ADP ribosylating activity of the M. penetrans toxin. Furthermore, the present invention provides a method of identifying a substance having the ability to inhibit the cytopathology-inducing activity of the M. penetrans toxin, comprising contacting the substance with the toxin or biologically active fragment thereof under conditions whereby cytopathology of target cells can be induced and detecting a decrease in the amount of cytopathology in the presence of the substance, as compared to a control amount of cytopathology in the absence of the substance, thereby identifying a substance having the ability to inhibit the cytopathology-inducing activity of the M. penetrans toxin or biologically active fragment thereof.

Substances identified in the screening assays of this invention to have the ability to inhibit or enhance various of the activities of the polypeptides and/or fragments of this invention can be employed in methods of diagnosing M. pneumoniae and/or M. penetrans infection, as well as in methods of treating and/or preventing M. pneumoniae and/or M. penetrans infection. For example, such substances can be present in a pharmaceutically acceptable carrier for administration to a subject and an effective amount of the substance can be administered to a subject to treat and/or prevent infection by Mycoplasma pneumoniae and/or M. penetrans.

It is also contemplated that the present invention includes methods of screening Mycoplasma pneumoniae and/or M. penetrans cultures for mutants defective in one or more of the biological activities of the CARDS toxin and/or M. penetrans toxin, for use in a vaccine preparation. Such mutants can be identified as having a defect in any of the biological activities of the CARDS toxin and/or M. penetrans toxin according to the protocols described herein and as are known in the art. Such mutants can be further tested for being attenuated in the ability to produce a clinical infection in a subject (i.e., for virulence potential) and then further evaluated for use as a vaccine according to known protocols.

For example, in one embodiment, CARDS toxin mutants of Mycoplasma pneumoniae (e.g., having a mutation in the CARDS coding sequence or lacking the CARDS coding sequence) and/or toxin mutants of M. penetrans can be generated through such art-known techniques as gene disruption and their virulence potential determined by challenge studies in hamsters and by adherence and cytopathology assessments in hamster tracheal rings in organ culture and in cell culture, as is well known in the art. In addition, complementation studies can be performed to restore the defective activity of the CARDS toxin and/or of the M. penetrans toxin, in order to characterize the mutant.

The present invention is more particularly described in the following examples, which are intended as illustrative only since numerous modifications and variations therein will be apparent to those skilled in the art.

### EXAMPLES

### Mycoplasma Strains and DNA Isolation Conditions

M. pneumoniae reference strain M129/B9 and clinical isolates S1, L2, JL1 and RJL1 were grown to late logarithmic phase in SP-4 medium at 37° C. for 72 h in 150-cm² tissue culture flasks. Mycoplasmas were harvested by washing three times with PBS [150 mM NaCl, 10 mM sodium phosphate, pH 7.4] and pelleting at 12,500 g for min at 4° C. A. pneumoniae chromosomal DNA was isolated using Easy DNA kit according to the manufacturer's protocol (Invitrogen).

### Mycoplasma Culture Conditions for Radiolabeling.

Wild-type Mycoplasma pneumoniae M129/B9 and clinical isolates were grown in SP-4 medium as above. Mycoplasma monolayers in logarithmic growth phase were washed two times with 10 ml PBS (pH 7.4) and one time with Dulbecco Modified Eagle Medium (DMEM) without L-cysteine and L-methionine and resuspended in 10 ml Dulbecco Modified Eagle Medium (DMEM) without L-cysteine and L-methionine supplemented with 10% heat-inactivated fetal bovine serum and 100 μCi L-[³⁵S]methionine. After 4 h incubation at 37° C., supernatants were removed and monolayers washed twice with 25 ml PBS. Mycoplasma cells were scraped into a volume of 10 ml sterile PBS, collected by centrifugation at 9,500×g and washed multiple times in PBS. Cell pellets were resuspended in 1 ml complete lysis buffer (CLB) prepared shortly before use (150 mM NaCl, 10 mM Tris, 20 μM EGTA, 0.5 M Triton-X 114, 1 mM CaCl₂ and protease inhibitors 1 μM pepstatin A, 200 μM PMSF, 1 mM N-α-p-tosyl-L-lysine chloromethyl ketone (TLCK), and 10 μM leupeptin. Cell pellets in CLB were sheared through 25 gauge needles using 3 ml syringes to obtain clear lysis. 20 μl aliquots of resuspended cell lysate were transferred to separate microfuge tubes for SDS-PAGE analysis and scintillation counter assessment (Beckman Instruments Inc. Irvine, Calif.). Radiolabeled lysates were diluted to 6 ml in CLB and passed through control and experimental SP-A columns (see below) in parallel.

### Purification of SP-A Binding Proteins

A 20×1.2 cm control glass column was packed with 3 ml uncoupled Sepharose, another identical (experimental) column was packed with 3 ml Sepharose coupled to SP-A. Coupling of SP-A to Sepharose CL-4B was performed as follows: A total of 1.5 mg of SP-A was coupled to 2 g of CNBr-activated Sepharose CL-4B according to the manufacture's instructions except the coupling buffer was 10 mM sodium bicarbonate, pH 8.3. SP-A coupled Sepharose was stored in 5 ml of 5 mM Tris pH 7.5, containing 1 mM NaN₃. Columns were equilibrated with 50 ml CLB prior to addition of radiolabeled cell lysates. Radiolabeled cell lysates were collected and reapplied to each column 3-4 times. After samples were added, columns were washed with 10 times volume of packed material to remove unbound proteins. M. pneumoniae SP-A-binding proteins were eluted using a NaCl gradient (0.2 to 3 M NaCl) containing 10 mM EDTA. Eluates were collected as 1 ml fractions, and 20 μl from each fraction was assayed for specific activity with a scintillation counter.

### SDS-PAGE and Autoradiogram.

Fractions eluted from columns were individually dialyzed/desalted against PBS and concentrated by an Amicon concentrator/lyophilizer to 1/30^{th} of original volume. Samples were resolved in 12% SDS-PAGE and stained with Coomassie brilliant blue or transferred to nitrocellulose and exposed to Kodak XRP-40X-ray film (Kodak, Rochester, N.Y.) for 4-8 days.

### MALDI-TOF Protein Sequencing.

SDS-polyacrylamide gels containing M. pneumoniae SP-A binding proteins were stained with Coomassie brilliant blue and washed thoroughly in distilled water. Individual protein bands were excised from acrylamide gels and subjected to MALDI-TOF by the microsequencing facility at Baylor College of Medicine (Houston, Tex.).

### Bacterial Strains, Plasmids and DNA Manipulations.

Escherichia coli INVαF′ [F′endA1rec1hsdR17supE44gyrA961acZM15 (lacZYAargF)] (Invitrogen) and E. coli BL21(DE3) [F'ompT hsds (r_{B}⁻m_{B}⁻) gal dcm λ(DE3) pLysS] were grown in Luria Bertani (LB) broth and used to clone and express mycoplasma CARDS toxin genes. For DNA manipulations, the following vectors were used: pCR2.1 (Ap^{r}, Km^{r}TA cloning vector [Invitrogen]) and pET19b (Ap^{r}, N-terminal His¹⁰ tag, expression vector [Novagen]). Plasmid DNA was purified using the QIAprep spin protocol according to the manufacturer (Qiagen).

### SOE-PCR

In attempting to determine precise binding motifs of M. pneumoniae SP-A binding proteins, both full size and truncated overexpressed proteins are employed. Initially, the number of truncated proteins will depend upon the number and location of UGA codons. Should the possibility arise that SP-A binding motifs are located in UGA-coded regions of a protein, this issue will be addressed using full-size proteins, or protease-digested peptide fragments, or synthetic peptides as described herein. UGA usage problems in genes encoding SP-A binding proteins, as well as other mycoplasma proteins, are known. In such proteins, the UGA codons in the corresponding genes are modified by site-directed mutagenesis to express full size proteins. PCR-based “splicing by overlap extension” (SOE) methods are employed to mutagenize UGA in these genes. This method is based on the principle that two overlapping complementary ends may prime on each other and be extended to yield a hybrid product, and a second PCR with two primers annealing at the non-overlapping ends will amplify this hybrid. An example of a stepwise strategy for SOE-PCR is as follows. 1. ‘a’ and ‘d’ are primers for a gene and ‘b’ and ‘c’ are primers to mutagenize the UGA region. 2. Amplification carried out with primers ‘a’ and ‘b’ and using genomic DNA as template gives a DNA fragment “AB” of the gene. 3. Amplification carried out with primers ‘c’ and ‘d’ and using genomic DNA as template will give DNA fragment “CD” of the gene. 4. Amplification with primers ‘a’ and ‘d’ and using DNA fragments “AB”+“CD” as templates will give the UGA modified mutant gene fragment. The overlapping primers covering the UGA codon in the genes are modified as UGG, a codon that still codes for tryptophan, and the primer sets depend upon the number of UGAs to be mutated in each gene. In all cases, genomic DNA of M. pneumoniae is used as template, and AccuTaq polymerase mix (Sigma) is used to amplify DNA fragments.

### Immunoblot Assay

Mycoplasma total proteins or purified recombinant CARDS protein were resolved on 4-12% SDS-polyacrylamide gels (NuPAGE, InVitrogen) (His-tag released, i.e., minus His tag) and transferred electrophoretically to nitrocellulose membranes (Towbin et al., 1979). Membranes were blocked for two hours with 5% (wt/vol) blotto [nonfat dry milk in TBS containing 0.1% Tween-20 (TBST)], followed by three washes with TBST, and incubated with M. pneumoniae infected patient sera (1:50 to 1:100 in 2% blotto) at RT for 2 h. Then, individual membranes were washed three times (15 min per wash) in TBST and incubated for 2 h (ambient temperature) with alkaline phosphatase-conjugated goat anti-human IgG Abs at a dilution of 1:2000 in TBST, which were washed 5 additional times with TBST, then color developed with BCIP/NBT tablets (Sigma).

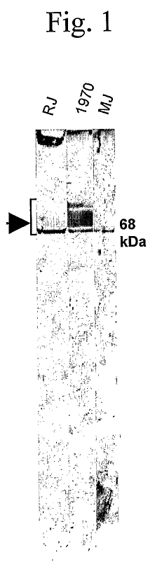

FIG. 1 is an immunoblot of sera from three patients, RJ, 1970 and MJ, infected with Mycoplasma pneumoniae. Purified M. pneumoniae recombinant CARDS toxin was resolved in 4-12% SDS-PAGE and transferred to nitrocellulose membranes. Membranes were blocked for two hours with 5% blotto and treated with patients' sera for two hours at room temperature. Patients' sera were diluted as follows. RJ and MJ: 1:50, and 1970:1:100 in 2% blotto. Membranes were washed and treated with alkaline phosphatase-conjugated goat anti-human antibodies diluted 1:2000 in TBS-T and two hours and color developed. Patients RJ and MJ died within about three weeks of infection and patient 1970 was hospitalized with mycoplasmal pneumonia and recovered. A 68 kDa MW recombinant CARDS toxin is indicated by the arrow; higher molecular weight and diffuse bands represent His-tagged subpopulation of recombinant CARDS toxin. Detection of antibodies to the CARDS toxin indicates in situ synthesis of CARDS toxin during infection and its immunogenicity.

Additional Studies on Patients Infected with M. pneumoniae.

In further studies, acute and convalescent sera were collected from patients with Mycoplasma pneumoniae-diagnosed respiratory infections that ranged from tracheobronchitis to bronchopneumonia. Two or three blood samples were obtained from each patient. The first blood sample was collected during the acute phase of the disease, approximately two weeks following exposure to M. pneumoniae. The second and third “convalescent” serum samples were obtained 14 and 28 days later, respectively. Control baseline serum samples were obtained from pregnant women attending the University of Texas Health Science Center at San Antonio OB-GYN clinic.

All serum samples were assessed by immunoblotting against total M. pneumoniae proteins. Specifically, to detect CARDS toxin protein in patients' sera, M pneumoniae total cell preparations of different clinical isolated (RJ1, J1, S1 and L2) and laboratory strain (B9) were dissolved in 1501 SDS sample buffer, boiled for two minutes and separated by SDS-PAGE using 4-12% NuPAGE SDS-polyacrylamide gels. Proteins were transferred to nitrocellulose membranes (Shleicher & Schull, Dassel, Germany) by electroblotting. Membranes were blocked for one hour at room temperature with blocking buffer (20 mM Tris-base, 150 mM NaCl, 3% skim milk powder) and incubated with anti-CARDS Toxin mouse polyclonal antibodies diluted 1:2000 in antibody buffer (20 mM Tris-base, 150 mM NaCl, 3% skim milk powder) for one hour at 37° C. Bound IgG was detected with alkaline phosphatase (AP)-conjugated goat-antimouse IgG diluted 1:3000. Membranes were developed for 1-5 minutes with nitroblue tetrazolium (NBT) and 5-bromo-4-chloro-3-indolyl phosphate p-toluidium (BCIP) solution. Results of the immunoblotting show a colored band of 68 kDa molecular weight on each membrane and thus demonstrate the presence of the CARDS toxin protein in each clinical isolate at concentrations that appear to vary among individuals.

Additional immunoblot analyses were carried out to detect antibodies to CARDS toxin in infected patients' sera wherein M. pneumoniae recombinant 68 kDa CARDS (rCARDS) toxin (3 μg) or the N terminal domain of CARDS toxin, rD1 (1 μg) as described herein was dissolved in 150 μl LDS sample buffer (NuPAGE), boiled for two minutes and separated by SDS-PAGE using 4-12% NuPAGE SDS-polyacrylamide gels. Proteins were transferred to nitrocellulose membranes (Schleicher & Schull, Dassel, Germany) by electroblotting and membranes were blocked for one hour at room temperature with blocking buffer (20 mM Tris-base, 150 mM NaCl, 3% skim milk powder). Membranes were cut into 3 mm strips and incubated with human serum samples diluted 1:200 in buffer (20 mM Tris-base, 150 mM NaCl, 3% skim milk powder) for one hour at 37° C. Serum samples were from M. pneumonia-infected patients designated patients 1 and 2 and the first serum samples were collected during the acute phase of disease (designated 1-1 and 2-1, respectively). The second serum samples (1-2 and 2-2) and third serum samples (1-3 and 2-3) were obtained 14 and 28 days later, respectively.

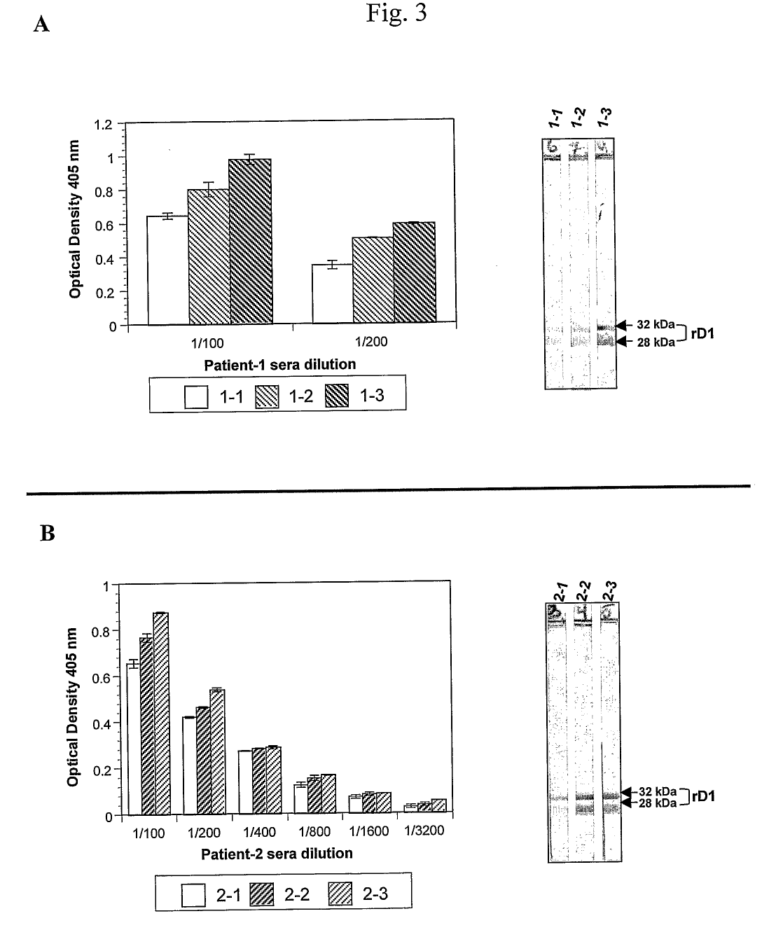

Bound IgG was detected with alkaline phosphatase (AP)-conjugated goat-antihuman IgG diluted 1:3000. Individual strips were developed for 1-5 minutes with nitroblue tetrazolium (NBT) and 5-bromo-4-chloro-3-indolyl phosphate p-toluidium (BCIP) solution. Results of the immunoblotting showed a colored band of 68 kDa molecular weight on each membrane containing rCARDs toxin and colored bands of 32 kDa and 28 kDa on each membrane containing the D1 domain, thus demonstrating seroconversion in these patients and detection of antibodies to the CARDS toxin, either as a recombinant protein or as the D1 domain. In the latter assay, the color intensity of each band appears to increase in the samples in a manner consistent with the time course of collection from the patient during the course of the disease (i.e., 1-1<1-2<1-3) (FIG. 3).

ELISAs were also carried out on the samples collected from patients 1 and 2 described above (i.e., samples 1-1, 1-2, 1-3, 2-1, 2-2, and 2-3). In these assays, washing at each stage was performed at least three times with PBS and sera and antibodies were diluted in 1% BSA in PBS. Each well of Immulon 4 HBX Immunoplates (Dynox) was coated overnight at 4° C. with 50 μl of rCARDS toxin/D1 (1 μg/well) diluted in carbonate/bicarbonate buffer (32 mM Na₂CO₃, 64 mM NaHCO₃). Individual plates were washed, 100 μl of 1 mg/ml (wt/vol) BSA in PBS was added to each well, and incubation continued for two hours at room temperature. After washing, 50 μl of diluted human serum samples (1/50 to 1/3200) were added to each well, and plates were incubated for two hours at room temperature. Then, plates were washed, and 50 μl of diluted (1:1000) alkaline phosphatase (AP)-conjugated goat-antihuman IgG (Zymed) were added to each well. Plates were incubated for 1.5 hours at room temperature, washed and 50 μl of substrate solution [p-nitrophenyl phosphate (PNPP)/0.1M Tris pH 9.6] was added and plates were incubated at room temperature for 30-60 minutes. Absorbance values at 450 nm were determined for each well.

The results for patient 1 with serum dilutions of 1/100 and 1/200 and rD1 as the antigen showed a decrease in optical density at the greater dilution of serum and a stepwise increase in optical density in the samples collected sequentially during the course of disease (i.e., 1-1<1-2<1-3) (FIG. 3A). This stepwise increase correlates with the increased color intensity observed with these serum samples in the immunoblot assay (FIG. 3A). Similar results were obtained with sequential serum samples from patient 1 when rCARDS Toxin was used as the antigen.

The results for patient 2 with serum dilutions of 1/100, 1/200, 1/400, 1/800, 1/1600 and 1/3200 and rD1 as the antigen showed a decrease in optical density as the dilution of serum increased and a stepwise increase in optical density in the samples collected sequentially during the course of disease (i.e., 2-1<2-2<2-3) (FIG. 3B). This stepwise increase correlates with the increased color intensity observed with these serum samples in the immunoblot assay (FIG. 3B). Similar results were obtained with sequential serum samples from patient 2 when rCARDS Toxin was used as the antigen.

Additional studies were conducted wherein each well of an Immulon 4 HBX Immunoplate (Dynox) was coated overnight at 4° C. with 50 μl of rCARDS toxin (1, 2 or 3 μg/well) diluted in carbonate/bicarbonate buffer. After washing, 50 μl of diluted human serum samples (1/200 dilution of convalescent serum 1-3 as described above) was added to each well and plates were incubated for two hours at room temperature prior to detection of bound IgG. Negative patient serum control was also included. The results showed an optical density around 1.8 and 1.9±SE for all three concentrations of rCARDS toxin and an optical density of the negative control around 0.6 and 0.7±SE for all concentrations of toxin.

A further study was carried out as described above, except that each well of Immulon 4 HBX Immunoplates (Dynox) was coated overnight at 4° C. with 50 μl of CARDS rD1 domain diluted as follows: 1, 2, 3, 4, 5 or 6 μg/well, in carbonate-bicarbonate buffer. Negative patient serum control was also included. The results show an optical density between 1.0 and 1.2±SE for all six concentrations of rD1 domain and an optical density of the negative control of 0.2±SE or less for all concentrations of rD1.

Overall, these immunoblot and ELISA studies demonstrate that both CARDS toxin and antibodies to CARDS toxin can be detected according to the methods of this invention and that the assays can be performed with as little as 1 μg of toxin either as the recombinant protein or as the D1 domain. These studies also indicate that the D1 domain may be a better target in an ELISA format, with lower background levels.

Identification of Mycoplasma pneumoniae by PCR in Sputum Samples using CARDS Toxin as a Target DNA Molecule

In this assay, phosphate buffered saline (PBS), with and without saliva, was mixed with a cell suspension (cells grown 2-3 days at 37° C. in SP-4 medium; total cells ˜1×10⁹) of M. pneumoniae S1 cells in a 1:1 ratio and centrifuged. The pellet was resuspended in 200 μl of water and incubated at 4° C. for 20 minutes. The sample was then boiled at 100° C. for 15 minutes. 37-40 μl of this sample was used for PCR in a total reaction volume of 50 μl. The samples were serially diluted 10⁻¹ to 10⁻⁹ in PBS. PCR conditions were 95° C. for five minutes; 94° C. for one minute; 55° C. for one minute; 72° C. for one minute and 72° C. for 10 minutes, for 30 cycles. The amplification primer set was Primer 12a forward: (nts. 1197-1220; 24 bp) 5′ gcttgttctggaataccaagagtg 3′ (SEQ ID NO:23) and Primer 15a reverse: (nts. 1541-1564; 24 bp) 5′ ccattctacccaatcccagctgta 3′ (SEQ ID NO:26). The product size of the amplicon was 368 base pairs. Detection was by ethidium bromide staining or autoradiography with a ³²P labeled probe. The probe used to detect the amplicon by autoradiography was Primer 14a forward: (nts 1371-1429; 59 bp) 5′ gctggtattggaggggttattactataccccacaattaagtggftggtcttatcagatg 3′ (SEQ ID NO:25). Results of this study demonstrate that M. pneumoniae nucleic acid can be detected in the presence or absence of saliva and that one mycoplasma cell can be identified using this primer/probe set.

### Cloning and Sequencing of CARDS

Based on the published genome sequence of M. pneumoniae M129/B9 (Himmelreich et al., 1996, SEQ ID NO:7), the complete open reading frame of cards was analyzed. Translation of nucleotide sequences to amino acids revealed the existence of eight TGA codons within the coding region of cards. Start and stop codons and the eight intervening TGA codons are indicated in bolded text.

tttttaattt gtaaaatttc attttttaaa aatgccaaat cctgttagat ttgtttaccg
tgttgatttg agaagccctg aagaaatttt tgaacatggc ttttcaactt taggtgatgt
gagaaatttc tttgaacaca ttctctccac taattttggt agaagctatt ttatttccac
ttcagaaaca cccacagcag ctattcgctt ctttggtagc tggttacggg aatatgtacc
agagcacccc agaagggctt acttatatga aattcgtgcc gaccaacact tttacaatgc
ccgcgccact ggggagaact tgttagattt aatgcgtcaa agacaagtag tatttgactc
tggtgatcga gaaatggcac aaatgggaat tagagcttta cgcacttcct ttgcgtatca
acgtgaatgg tttaccgatg gtccaattgc agcagctaat gtccgtagtg cttgactagt
agatgctgtt cccgttgaac ctggtcatgc tcaccacccg gctggtcgtg ttgtagagac
tactagaatt aatgaaccgg aaatgcacaa ccctcattat caagagctgc aaacccaagc
caatgatcaa ccatgattgc caacaccagg aatagctact cctgtacatt tatcaattcc
ccaagcagct tccgttgctg atgtttcgga aggtacttcc gcttcgctat cgtttgcgtg
ccctgattga agtccacctt ctagtaatgg tgaaaatccg ctagacaaat gcattgcgga
aaagattgat aactataacc tacaatcctt accacagtac gctagcagtg taaaggaact
ggaagataca ccagtatacc taaggggaat taaaacgcaa aaaaccttta tgttacaagc
agatccgcaa aataacaatg tctttttggt cgaagtaaac cccaaacaaa agtccagctt
tccccaaacc atcttctttt gggatgttta tcaacgaatt tgtctcaagg atttaactgg
tgcacaaatc agtctttcgc ttactgcctt tactactcag tatgctggtc agctcaaagt
gcaccttagt gttagcgcgg ttaatgccgt gaaccaaaag tgaaaaatga caccgcaaga
cattgcaata actcagtttc gggtctcctc tgaactgtta ggtcaaactg aaaatggctt
gttctgaaat accaagagtg gtggttcaca acacgatttg tatgtatgtc ctttgaaaaa
tccacctagt gatttggaag aattacaaat aattgttgat gaatgtacta cccatgcgca
gtttgttact atgcgtgcag ctagcacctt ctttgttgat gttcagctag gctggtattg
aaggggttat tactataccc cacaattaag tggttgatct tatcagatga aaacaccaga
tggacagata ttctatgatc taaaaacttc gaaaatcttc tttgtccagg acaaccaaaa
cgtgttcttt ctccataata aactcaacaa acaaactggt tacagctggg attgagtaga
atggctaaaa catgacatga atgaggacaa agacgaaaac tttaaatggt acttttcgcg
tgatgacctt accattcctt ccgttgaagg gcttaacttc cgccacattc gctgttacgc
tgacaaccag cagttaaagg tgatcataag cggttcacgt tggggcggtt ggtactccac
ttacgataaa gttgaaagta atgtcgaaga taagattttg gtcaaagatg gttttgatcg
cttttagcga ttaagcttta acgtcactgt tttgctctaa tgttagaagc aaagatcttg

The entire cards sequence was amplified using forward primer 5′-tttttacatatgccaaatcctgtt-3′ (primer 1, SEQ ID NO:12) and reverse primer 5′-gatcgcttttagcggatcctttaacg-3′ (primer 2, SEQ ID NO:64), which produces NdeI and BamHI (underlined) sites at 5′ and 3′ ends of the cards ORF, respectively. Both fragments were ligated into the pCR 2.1 vector and transformed into E. coli INVαF′ cells for automated sequencing using M13 forward and reverse primers.

Site-directed mutagenesis of the cards gene to permit expression of total recombinant CARDS protein was necessary, which required the correction of TGAs to TGGs in order to encode tryptophan in E. coli. Therefore, specific primers were designed as indicated below. Primers below are also used to generate specific CARDS domains for generating specific antibody probes.

CARDS: Oligonucleotide Sequences within Selected (Above) Nucleotide Sequence.

Pri 1-16: Modified oligonucleotide sequence* to amplify the cards sequence. * modified nucleotides are given in bold. Complementary oligonucleotide sequence are given underneath the reverse primers (2, 3, 5, 7, 9, 11, 13 and 15)

| | | | | |
|---|---|---|---|---|
| MPN372: | 23 | tttttaaaaatgccaaatcctgtt | 46 | (SEQ ID NO: 28) |
| | | \|\|\|\|\|\| \| \|\|\|\|\|\|\|\|\|\|\|\|\|\|\| | | |
| Pri-1: | 1 | tttttacatatgccaaatcctgtt | 24 | (SEQ ID NO: 12) |
| MPN372: | 458 | aatgtccgtagtgcttgact | 477 | (SEQ ID NO: 29) |
| | | \|\|\|\|\|\|\|\|\|\|\|\|\|\|\|\|\| \|\| | | |
| Pri-3: | 20 | aatgtccgtagtgcttggct | 1 | (SEQ ID NO: 30) |
| | | ttacaggcatcacgaaccga | | (SEQ ID NO: 31) |
| MPN372: | 469 | tgcttgactagtagatgctgtt | 490 | (SEQ ID NO: 32) |
| | | \|\|\|\|\|\| \|\|\|\|\|\|\|\|\|\|\|\|\|\|\| | | |
| Pri-4 | 1 | tgcttggctagtagatgctgtt | 22 | (SEQ ID NO: 33) |
| MPN372: | 613 | atgattgccaacaccagg | 630 | (SEQ ID NO: 34) |
| | | \|\|\| \|\|\|\|\|\|\|\|\|\|\|\|\|\| | | |
| Pri-5: | 18 | atggttgccaacaccagg | 1 | (SEQ ID NO: 35) |
| | | taccaacggttgtggtcc | | (SEQ ID NO: 36) |
| MPN372: | 610 | accatgattgccaacacc | 627 | (SEQ ID NO: 37) |
| | | \|\|\|\|\|\| \|\|\|\|\|\|\|\|\|\|\| | | |
| Pri-6: | 1 | accatggttgccaacacc | 18 | (SEQ ID NO: 38) |
| MPN372: | 722 | cctgattgaagtccacctt | 740 | (SEQ ID ND: 39) |
| | | \|\|\|\|\|\|\|\| \|\|\|\|\|\|\|\|\|\| | | |
| Pri-7: | 19 | cctgattggagtccacctt | 1 | (SEQ ID NO: 40) |
| | | ggactaacctcaggtggaa | | (SEQ ID NO: 41) |
| MPN372: | 717 | cgtgccctgattgaagtc | 734 | (SEQ ID NO: 42) |
| | | \|\|\|\|\|\|\|\|\|\|\|\|\| \|\|\|\| | | |
| Pri-8: | 1 | cgtgccctgattggagtc | 18 | (SEQ ID NO: 43) |
| MPN372: | 1117 | aaagtgaaaaatgacaccgc | 1136 | (SEQ ID NO: 44) |
| | | \|\|\|\|\|\| \|\|\|\|\|\|\|\|\|\|\|\|\| | | |
| Pri-9: | 20 | aaagtgaaaaatgacaccgc | 1 | (SEQ ID NO: 45) |
| | | tttcaccttttactgtggcg | | (SEQ ID NO: 46) |
| MPN372: | 1115 | caaaagtgaaaaatgacacc | 1134 | (SEQ ID NO: 47) |
| | | \|\|\|\|\|\|\|\| \|\|\|\|\|\|\|\|\|\|\| | | |
| Pri-10: | 1 | caaaagtggaaaatgacacc | 20 | (SEQ ID NO: 48) |
| MPN372: | 1192 | aaatggcttgttctgaaatacc | 1213 | (SEQ ID NO: 49) |
| | | \|\|\|\|\|\|\|\|\|\|\|\|\|\|\| \|\|\|\|\|\| | | |
| Pri-11: | 22 | aaatggcttgttctggaatacc | 1 | (SEQ ID NO: 50) |
| | | tttaccgaacaagaccttatgg | | (SEQ ID NO: 22) |
| MPN372: | 1197 | gcttgttctgaaataccaagagt | 1219 | (SEQ ID NO: 51) |
| | | \|\|\|\|\|\|\|\|\|\| \|\|\|\|\|\|\|\|\|\|\|\| | | |
| Pri-12: | 1 | gcttgttctggaataccaagagt | 23 | (SEQ ID NO: 52) |
| MPN372: | 1368 | taggctggtattgaaggggt | 1387 | (SEQ ID NO: 53) |
| | | \|\|\|\|\|\|\|\|\|\|\|\|\| \|\|\|\|\|\| | | |
| Pri-13: | 20 | taggctggtattggaggggt | 1 | (SEQ ID NO: 54) |
| | | atcagaccataacctcccca | | (SEQ ID NO: 55) |
| MPN372: | 1374 | ggtattgaaggggttattactataccccacaattaagtggttgatcttatcagatg | 1429 | (SEQ ID NOS: 56 and 57) |
| | | \|\|\|\|\|\|\| \|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\| | | |
| Pri-14: | 1 | ggtattggaggggttattactataccccacaattaagtggttggtcttatcagatg | 56 | |
| MPN372: | 1541 | tacagctgggattgagtagaa | 1561 | (SEQ ID NO: 58) |
| | | \|\|\|\|\|\|\|\|\|\|\|\|\|\| \|\|\|\|\|\| | | |
| Pri-15: | 21 | tacagctgggattgggtagaa | 1 | (SEQ ID NO: 59) |
| | | atgtagaccctaacccatctt | | (SEQ ID NO: 60) |
| MPN372: | 1541 | tacagctgggattgagtagaa | 1561 | (SEQ ID NO: 61) |
| | | \|\|\|\|\|\|\|\|\|\|\|\|\|\| \|\|\|\|\|\| | | |
| Pri-16: | 1 | tacagctgggattgggtagaa | 21 | (SEQ ID NO: 62) |
| MPN372: | 1796 | gatcgcttttagcgattaagctttaacg | 1824 | (SEQ ID NO: 63) |
| | | \|\|\|\|\|\|\|\|\|\|\|\|\|\|\| \| \|\|\|\|\|\|\|\| | | |
| Pri-2: | 28 | gatcgcttttagcgaggatcctttaacg | 1 | (SEQ ID NO: 64) |
| | | ctagcgaaaatcgctcctaggaaattgc | | (SEQ ID NO: 13) |

Sequence of M. pneumoniae CARDS.

The cards gene of M. pneumoniae reference strain M129/B9 and clinical 50 isolates (S1, L2, JL and RJL1) were cloned in a PCRII vector individually and sequenced.

M129/B9 represents the reference strain and S1, L2, RJL1 and JL are clinical isolates from patients in San Antonio and Dallas.

All clinical isolates have the same mutation at nucleotide 1112^{(T→G)} from the ATG start codon, which differs from the published reference strain. However, in clinical isolate S1 three additional nucleotide changes occur at nucleotide base positions 113^{(T→C)}, 922^{(T→C)} and 1172^{(T→C)}.

The following nucleotide changes were detected in the other clinical isolates:

### L2: 734^{(A→G)} and 1112^{(T→G)}.

### JL: 1112^{(T→G)}.

### RJL1: 1112^{(T→G)} and 1174^{(T→C)}.

Coding Sequence of S1 (Mycoplasma pneumoniae Clinical Isolate)

Bolded gs shown were introduced by site directed mutagenesis in order to express CARDS protein in E. coli.

| S1 Nucleotide sequence (SEQ ID NO: 8) | |
|---|---|
| atgccaaatc ctgttagatt tgtttaccgt gttgatttga gaagccctga agaaattttt | 60 |
| gaacatggct tttcaacttt aggtgatgtg agaaatttct ttgaacacat tccctccact | 120 |
| aattttggta gaagctattt tatttccact tcagaaacac ccacagcagc tattcgcttc | 180 |
| tttggtagct ggttacggga atatgtacca gagcacccca gaagggctta cttatatgaa | 240 |
| attcgtgccg accaacactt ttacaatgcc cgcgccactg gggagaactt gttagattta | 300 |
| atgcgtcaaa gacaagtagt atttgactct ggtgatcgag aaatggcaca aatgggaatt | 360 |
| agagctttac gcacttcctt tgcgtatcaa cgtgaatggt ttaccgatgg tccaattgca | 420 |
| gcagctaatg tccgtagtgc ttggctagta gatgctgttc ccgttgaacc tggtcatgct | 480 |
| caccacccgg ctggtcgtgt tgtagagact actagaatta atgaaccgga aatgcacaac | 540 |
| cctcattatc aagagctgca aacccaagcc aatgatcaac catggttgcc aacaccagga | 600 |
| atagctactc ctgtacattt atcaattccc caagcagctt ccgttgctga tgtttcggaa | 660 |
| ggtacttccg cttcgctatc gtttgcgtgc cctgattgga gtccaccttc tagtaatggt | 720 |
| gaaaatccgc tagacaaatg cattgcggaa aagattgata actataacct acaatcctta | 780 |
| ccacagtacg ctagcagtgt aaaggaactg gaagatacac cagtatacct aaggggaatt | 840 |
| aaaacgcaaa aaacctttat gttacaagca gatccgcaaa ataacaatgt ctttttggtc | 900 |
| gaagtaaacc ccaaacaaaa gcccagcttt ccccaaacca tcttcttttg ggatgtttat | 960 |
| caacgaattt gtctcaagga tttaactggt gcacaaatca gtctttcgct tactgccttt | 1020 |
| actactcagt atgctggtca gctcaaagtg caccttagtg ttagcgcggt taatgccgtg | 1080 |
| aaccaaaagt ggaaaatgac accgcaagac agtgcaataa ctcagtttcg ggtctcctct | 1140 |
| gaactgttag gtcaaactga aaatggcttg tcctggaata ccaagagtgg tggttcacaa | 1200 |
| cacgatttgt atgtatgtcc tttgaaaaat ccacctagtg atttggaaga attacaaata | 1260 |
| attgttgatg aatgtactac ccatgcgcag tttgttacta tgcgtgcagc tagcaccttc | 1320 |
| tttgttgatg ttcagctagg ctggtattgg aggggttatt actatacccc acaattaagt | 1380 |
| ggttggtctt atcagatgaa aacaccagat ggacagatat tctatgatct aaaaacttcg | 1440 |
| aaaatcttct ttgtccagga caaccaaaac gtgttctttc tccataataa actcaacaaa | 1500 |
| caaactggtt acagctggga ttgggtagaa tggctaaaac atgacatgaa tgaggacaaa | 1560 |
| gacgaaaact ttaaatggta cttttcgcgt gatgacctta ccattccttc cgttgaaggg | 1620 |
| cttaacttcc gccacattcg ctgttacgct gacaaccagc agttaaaggt gatcataagc | 1680 |
| ggttcacgtt ggggcggttg gtactccact tacgataaag ttgaaagtaa tgtcgaagat | 1740 |
| aagattttgg tcaaagatgg ttttgatcgc ttt | 1773 |

Below are the amino acid sequences of individual clinical isolates.

JL (SEQ ID NO: 3)
MPNPVRFVYR VDLRSPEEIF EHGFSTLGDV RNFFEHILST
NFGRSYFIST SETPTAAIRF
FGSWLREYVP EHPRRAYLYE IRADQHFYNA RATGENLLDL
MRQRQVVFDS GDREMAQMGI
RALRTSFAYQ REWFTDGPIA AANVRSAWLV DAVPVEPGHA
HHPAGRVVET TRINEPEMHN
PHYQELQTQA NDQPWLPTPG IATPVHLSIP QAASVADVSE
GTSASLSFAC PDWSPPSSNG
ENPLDKCIAE KIDNYNLQSL PQYASSVKEL EDTPVYLRGI
KTQKTFMLQA DPQNNNVFLV
EVNPKQKSSF PQTIFFWDVY QRICLKDLTG AQISLSLTAF
TTQYAGQLKV HLSVSAVNAV
NQKWKMTPQD SAITQFRVSS ELLGQTENGL FWNTKSGGSQ
HDLYVCPLKN PPSDLEELQI
IVDECTTHAQ FVTMRAASTF FVDVQLGWYW RGYYYTPQLS
GWSYQMKTPD GQIFYDLKTS
KIFFVQDNQN VFFLHNKLNK QTGYSWDWVE WLKHDMNEDK
DENFKWYFSR DDLTIPSVEG
LNFRHIRCYA DNQQLKVIIS GSRWGGWYST YDKVESNVED
KILVKDGFDR F*
RJL1 (SEQ ID NO: 4)
MPNPVRFVYR VDLRSPEEIF EHGFSTLGDV RNFFEHILST
NFGRSYFIST SETPTAAIRF
FGSWLREYVP EHPRRAYLYE IRADQHFYNA RATGENLLDL
MRQRQVVFDS GDREMAQMGI
RALRTSFAYQ REWFTDGPIA AANVRSAWLV DAVPVEPGHA
HHPAGRVVET TRINEPEMHN
PHYQELQTQA NDQPWLPTPG IATPVHLSIP QAASVADVSE
GTSASLSFAC PDWSPPSSNG
ENPLDKCIAE KIDNYNLQSL PQYASSVKEL EDTPVYLRGI
KTQKTFMLQA DPQNNNVFLV
EVNPKQKSSF PQTIFFWDVY QRICLKDLTG AQISLSLTAF
TTQYAGQLKV HLSVSAVNAV
NQKWKMTPQD SAITQFRVSS ELLGQTENGL FRNTKSGGSQ
HDLYVCPLKN PPSDLEELQI
IVDECTTHAQ FVTMRAASTF FVDVQLGWYW RGYYYTPQLS
GWSYQMKTPD GQIFYDLKTS
KIFFVQDNQN VFFLHNKLNK QTGYSWDWVE WLKHDMNEDK
DENFKWYFSR DDLTIPSVEG
LNFRHIRCYA DNQQLKVIIS GSRWGGWYST YDKVESNVED
KILVKDGFDR F*
L2 (SEQ ID NO: 5)
MPNPVRFVYR VDLRSPEEIF EHGFSTLGDV RNFFEHILST
NFGRSYFIST SETPTAAIRF
FGSWLREYVP EHPRRAYLYE IRADQHFYNA RATGENLLDL
MRQRQVVFDS GDREMAQMGI
RALRTSFAYQ REWFTDGPIA AANVRSAWLV DAVPVEPGHA
HHPAGRVVET TRINEPEMHN
PHYQELQTQA NDQPWLPTPG IATPVHLSIP QAASVADVSE
GTSASLSFAC PDWSPPSSNG
ENPLGKCIAE KIDNYNLQSL PQYASSVKEL EDTPVYLRGI
KTQKTFMLQA DPQNNNVFLV
EVNPKQKSSF PQTIFFWDVY QRICLKDLTG AQISLSLTAF
TTQYAGQLKV HLSVSAVNAV
NQKWKMTPQD SAITQFRVSS ELLGQTENGL FWNTKSGGSQ
HDLYVCPLKN PPSDLEELQI
IVDECTTHAQ FVTMRAASTF FVDVQLGWYW RGYYYTPQLS
GWSYQMKTPD GQIFYDLKTS
KIFFVQDNQN VFFLHNKLNK QTGYSWDWVE WLKHDMNEDK
DENFKWYFSR DDLTIPSVEG
LNFRHIRCYA DNQQLKVIIS GSRWGGWYST YDKVESNVED
KILVKDGFDR F*
S1 (SEQ ID NO: 2)
MPNPVRFVYR VDLRSPEEIF EHGFSTLGDV RNFFEHIPST
NFGRSYFIST SETPTAAIRF
FGSWLREYVP EHPRRAYLYE IRADQHFYNA RATGENLLDL
MRQRQVVFDS GDREMAQMGI
RALRTSFAYQ REWFTDGPIA AANVRSAWLV DAVPVEPGHA
HHPAGRVVET TRINEPEMHN
PHYQELQTQA NDQPWLPTPG IATPVHLSIP QAASVADVSE
GTSASLSFAC PDWSPPSSNG
ENPLDKCIAE KIDNYNLQSL PQYASSVKEL EDTPVYLRGI
KTQKTFMLQA DPQNNNVFLV
EVNPKQKPSF PQTIFFWDVY QRICLKDLTG AQISLSLTAF
TTQYAGQLKV HLSVSAVNAV
NQKWKMTPQD SAITQFRVSS ELLGQTENGL SWNTKSGGSQ
HDLYVCPLKN PPSDLEELQI
IVDECTTHAQ FVTMRAASTF FVDVQLGWYW RGYYYTPQLS
GWSYQMKTPD GQIFYDLKTS
KIFFVQDNQN VFFLHNKLNK QTGYSWDWVE WLKHDMNEDK
DENFKWYFSR DDLTIPSVEG
LNFRHIRCYA DNQQLKVIIS GSRWGGWYST YDKVESNVED
KILVKDGFDR F*

These sequence data are summarized below.
1. Translation of the nucleotide sequence of the clinical isolates showed changes in amino acid positions at 38, 245, 308, 371, 391 and 392.
2. All the clinical isolates have changes at amino acid position 371^{Ile→Ser}.
3. JL had only one change at aa position 371^{Ile→Ser}.
4. RJL1 had one more additional change (comparing to JL) at aa position 392^{Trp→Arg}.
5. L2 had one more additional change (comparing to JL) at aa position 245^{Asp→Gly}.
6. S1 had three additional changes (comparing to JL) at aa positions 38^{Leu→Pro}, 308^{Ser→Pro} and 391^{Phe→Ser}.

### Expression and Purification of Recombinant CARDS Protein.

DNA fragments were generated by digesting plasmid pCR-cards with NdeI and BamHI and ligated into pET19b to generate pET-cards. The plasmid was transformed into competent E. coli BL21 (DE3) cells grown to a density of 2×10⁹ cells/ml at 37° C. in standard LB broth containing 100 μg/ml ampicillin (Sigma-Aldrich). Induction of recombinant protein synthesis was accomplished by addition of 100 μM of isopropyl thio β-galactopyranoside (Sigma-Aldrich), and bacteria were incubated for 3 h at 37° C. under aeration at 220 rpm. Cells from 1 ml samples were pelleted, resuspended in 250 μl of sample buffer (4% SDS, 125 mM Tris [pH 6.8], 10% β-ME, 10% glycerol, 0.2% bromophenol blue), and heated to 95° C. for 5 min. 10 μl aliquots of test samples were analyzed on 12% SDS/polyacrylamide gels. Recombinant colonies were screened for resistance to ampicillin and expression of a protein product of the correct size, and one recombinant clone from each construct was selected for further study. Verification of specific clones was achieved by restriction digestion and limited DNA sequencing. Fusion proteins were purified from recombinant E. coli under native condition by nickel affinity chromatography using the manufacturer's protocol (Qiagen).

### Preparation of Antisera Against Recombinant Mycoplasma Proteins.

Mice were immunized subcutaneously with 50-100 μg of recombinant total CARDS protein suspended in complete Freund's adjuvant (no peptides or truncated domains). Individual mice were boosted three times with the same amount of recombinant antigen in incomplete Freund's adjuvant at 14-day intervals. Serum samples were collected and used for immunological characterization. Monoclonal antibodies were produced using recombinant CARDS toxin and hybridoma supernatants were screened for immunoreactivity with CARDS protein and truncated peptides.

Full length recombinant CARDS Toxin (rTOX) and the amino terminal D1 domain of recombinant CARDS Toxin (rD1) were separated on 4-12% preparative gels, transferred to nitrocellulose and reacted with various concentrations (1:2, 1:10 and 1:50 or 1:100) of primary mouse antibodies against rTOX or rD1 (Monoclonal antibodies 11D1-2H10, isotype 1gGg1 and monoclonal antibody 19C4-2G10-1E1-2B9, isotype IgG3). Membranes were washed and reacted with alkaline phosphatase-conjugated goat anti-mouse IgG. Blots were washed again, followed by color development with NBT-BCIP reagent. Both antibodies bound a protein of approximately 70 kDa MW in membranes containing rTOX and both antibodies bound peptides of 28 kDa MW and 32 kDa MW in membranes containing rD1.

### Primers Designed to Express Specific Domains of CARDS

Introduced restriction sites are indicated by underline. Changes in nucleotide sequences are given in bold.

(SEQ ID NO: 12)

| | |
|---|---|
| tttttacatatgccaaatcctgtt | Primer 1 |

(SEQ ID NO: 72)

| | |
|---|---|
| tttttacatatgccaaatcctgttag | Primer 1a |

(SEQ ID NO: 65)

| | |
|---|---|
| ggatcctctacgcaatgcatttgtctag | 372D1R |

(SEQ ID NO: 66)

| | |
|---|---|
| catatgccaacaccaggaatagctactc | 372D2F |

(SEQ ID NO: 67)

| | |
|---|---|
| ggatccactaccagcctagctgaac . . . | 372D2R |

(SEQ ID NO: 68)

| | |
|---|---|
| catatgggtcagctcaaagtgcaccttag | 372D3F |

(SEQ ID NO: 64)

| | |
|---|---|
| gatcgcttttagcgaggatcctttaacg | Primer 2 |

| Amplified region of CARDS toxin nucleic acid encoding D1 | |
|---|---|
| 1 (SEQ ID NO: 74) | |
| atgccaaatc ctgttagatt tgtttaccgt gttgatttga gaagccctga agaaattttt | 60 |
| gaacatggct tttcaacttt aggtgatgtg agaaatttct ttgaacacat tctctccact | 120 |
| aattttggta gaagctattt tatttccact tcagaaacac ccacagcagc tattcgcttc | 180 |
| tttggtagct ggttacggga atatgtacca gagcacccca gaagggctta cttatatgaa | 240 |
| attcgtgccg accaacactt ttacaatgcc cgcgccactg gggagaactt gttagattta | 300 |
| atgcgtcaaa gacaagtagt atttgactct ggtgatcgag aaatggcaca aatgggaatt | 360 |
| agagctttac gcacttcctt tgcgtatcaa cgtgaatggt ttaccgatgg tccaattgca | 420 |
| gcagctaatg tccgtagtgc ttggctagta gatgctgttc ccgttgaacc tggtcatgct | 480 |
| caccacccgg ctggtcgtgt tgtagagact actagaatta atgaaccgga aatgcacaac | 540 |
| cctcattatc aagagctgca aacccaagcc aatgatcaac catggttgcc aacaccagga | 600 |
| atagctactc ctgtacattt atcaattccc caagcagctt ccgttgctga tgtttcggaa | 660 |
| ggtacttccg cttcgctatc gtttgcgtgc cctgattgga gtccaccttc tagtaatggt | 720 |
| gaaaatccgc tagacaaatg cattgcg | 747 |

Domains Expected to be Expressed in E. coli Using the Above Primers.

Overlapping amino acids within domains are indicated by underline.

| Domain 1 (SEQ ID NO: 69): Primer 1 and 372D1R | |
|---|---|
| 1 | MPNPVRFVYR VDLRSPEEIF EHGFSTLGDV RNFFEHILST NFGRSYFIST |
| 51 | SETPTAAIRF FGSWLREYVP EHPRRAYLYE IRADQHFYNA RATGENLLDL |
| 101 | MRQRQVVFDS GDREMAQMGI RALRTSFAYQ REWFTDGPIA AANVRSAWLV |
| 151 | DAVPVEPGHA HHPAGRVVET TRINEPEMHN PHYQELQTQA NDQ [symbol] |
| 201 | IATPVHLSIP QAASVADVSE GTSASLSFAC [symbol] ENPLDKCIA |
| | Theoretical pI/Mw: 5.54/28127.37 |
| Domain 1 with His tag (underlined) (SEQ ID NO: 75) | |
| | MGHHHHHHHHHHSSGHIDDDDKH |
| 1 | MPNPVRFVYR VDLRSPEEIF EHGFSTLGDV RNFFEHILST NFGRSYFIST |
| 51 | SETPTAAIRF FGSWLREYVP EHPRRAYLYE IRADQHFYNA RATGENLLDL |
| 101 | MRQRQVVFDS GDREMAQMGI RALRTSFAYQ REWFTDGPIA AANVRSAWLV |
| 151 | DAVPVEPGHA HHPAGRVVET TRINEPEMHN PHYQELQTQA NDQ [symbol] |
| 201 | IATPVHLSIP QAASVADVSE GTSASLSFAC [symbol] ENPLDKCIA |
| | Theoretical pI/Mw with the tag: 5.95/30894.20 |
| Domain 2: (SEQ ID NO: 70) 372D2F and 372D2R | |
| | [symbol] |
| 201 | IATPVHLSIP QAASVADVSE GTSASLSFAC [symbol] ENPLDKCIAE |
| 251 | KIDNYNLQSL PQYASSVKEL EDTPVYLRGI KTQKTFMLQA DPQNNNVFLV |
| 301 | EVNPKQKSSF PQTIFFWDVY QRICLKDLTG AQISLSLTAF TTQY AGQLKV |
| 351 | HLSVSAVNAV [symbol] IAITQFRVSS ELLGQTENGL [symbol] |
| 401 | HDLYVCPLKN PPSDLEELQI IVDECTTHAQ FVTMRAASTF FVDVQLGWY |
| | Theoretical pI/Mw: 5.05/28378.10 |
| Domain 3 (SEQ ID NO: 71): 372D3F and Primer 2 | |
| | AGQLKV |
| 351 | HLSVSAVNAV [symbol] IAITQFRVSS ELLGQTENGL [symbol] |
| 401 | HDLYVCPLKN PPSDLEELQI IVDECTTHAQ FVTMRAASTF FVDVQLGWYW |
| 451 | RGYYYTPQLS GWSYQMKTPD GQIFYDLKTS KIFFVQDNQN VFFLHNKLNK |
| 501 | QTGYSWDWVE WLKHDMNEDK DENFKWYFSR DDLTIPSVEG LNFRHIRCYA |
| 551 | DNQQLKVIIS GSRWGGWYST YDKVESNVED KILVKDGFDR F |
| | Theoretical pI/Mw: 5.69/28966.52 |

### Production of Recombinant N Terminal Domain of CARDS Toxin rD1

To produce rD1, the D1 PCR fragment (SEQ ID NO:74) encoding the cards first 249 amino acids (SEQ ID NO:69) was cloned into the E. coli His¹⁰-tagged expression vector, pET19b (Novagen), using Nde1 and BamHI restriction sites incorporated into the oligonucleotide primers used to amplify this nucleic acid 5′ tttttacatatgccaaatcctgttag 3′ (SEQ ID NO:72) and 5′ ggatcctctacgcaatgcatttgtctag 3′ (SEQ ID NO:65). Because the NdeI site in the vector overlaps an ATG start codon, cloning the D1 fragment into this site places the fragment in perfect register with the vector-derived His-tagged ribosome binding site. The amino acid sequence of the expressed protein with the His tag is shown in SEQ ID NO:75.

After cloning the D1 PCR fragment into pET19b and confirming the identify of the cloned fragment by DNA sequencing, a recombinant plasmid was used to transform E. coli strain BL21 (2DE3). Transformants were grown to mid-log phase before inducing D1 expression by addition of IPTG to a final concentration of 1 mM. After four hours, cells were harvested by centrifugation at 8000 g for 15 minutes at 4° C. and the pellet was resuspended in 50 mM phosphate buffer ph 8.0, containing 300 mM NaCl, 10 mM imidazole and complete, EDTA-free protease inhibitor (Sigma). Cells were disrupted by sonication; cellular debris and membranes were pelleted by centrifugation at 16000 g for 30 minutes and discarded; the supernatant was mixed with Ni-NTA agarose slurry and left on a rocker at room temperature for one hour; and then the slurry was loaded into a column. The Ni-NTA agarose packed column was extensively washed with 10 mM imidazole, 20 mM imidazole, and 50 mM imidazole in the same buffer used for pellet resuspension. Finally, D1 was purified in a single step elution with 250 mM imidazole in the same buffer. Fractions containing purified protein were desalted using P10 columns (Amersham Biosciences) with TG buffer (20 mM Tris-Cl, pH 7.4, 5% glycerol) and concentrated using YM-10 (Amicon) membranes. Protein concentrations were estimated using a BCA protein assay kit (Pierce) and the protein was aliquoted and stored at −80° C.

ADP-Ribosylation of G Proteins Following Incubation of CARDS Protein with HEp-2 Cells.

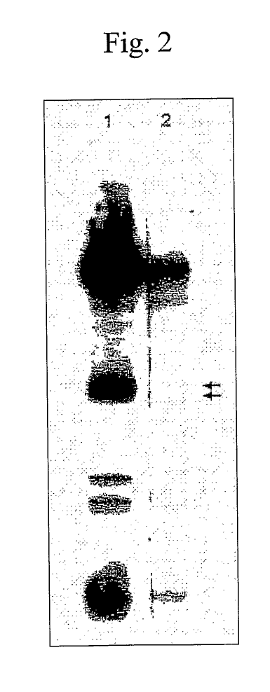

Confluent HEp-2 cells were incubated with medium alone or in the presence of 40 μg/ml CARDS protein for 16 hours at 37° C. Cells were washed and incubated with fresh medium at 37° C. for four hours. Cell free extracts (CFE) were prepared and assayed for ADP-ribosylation (CFE were incubated with 40 μg/ml CARDS protein for one hour with 0.1 μM [³²P]NAD in 100 mM Tris pH 7.5, 20 mM DTT). The reaction mixture was precipitated with 10% TCA and proteins were resolved in a 4-15% gradient gel by SDS-PAGE and transferred to nitrocellulose membrane for autoradiography. As shown in the autoradiogram in FIG. 2, the CARDS toxin exhibits ADP ribosylating activity.

### Cytopathology in Chinese Hamster Ovary (CHO) Cells

Cells were seeded into 25 cm² cell culture flasks and incubation was continued until monolayer confluence was achieved. Then, recombinant CARDS protein (20 μg/ml or 40 μg/ml) was added for 24 hours. Monolayers were photographed on an Olympus CK40 microscope at 20× magnification.

In CHO cells, the recombinant toxin causes cytopathology with an associated “foamy” appearance, rounding of cells and cell detachment from monolayers.

### Further Studies on Effect of CARDS Toxin in Mammalian Cell Cultures

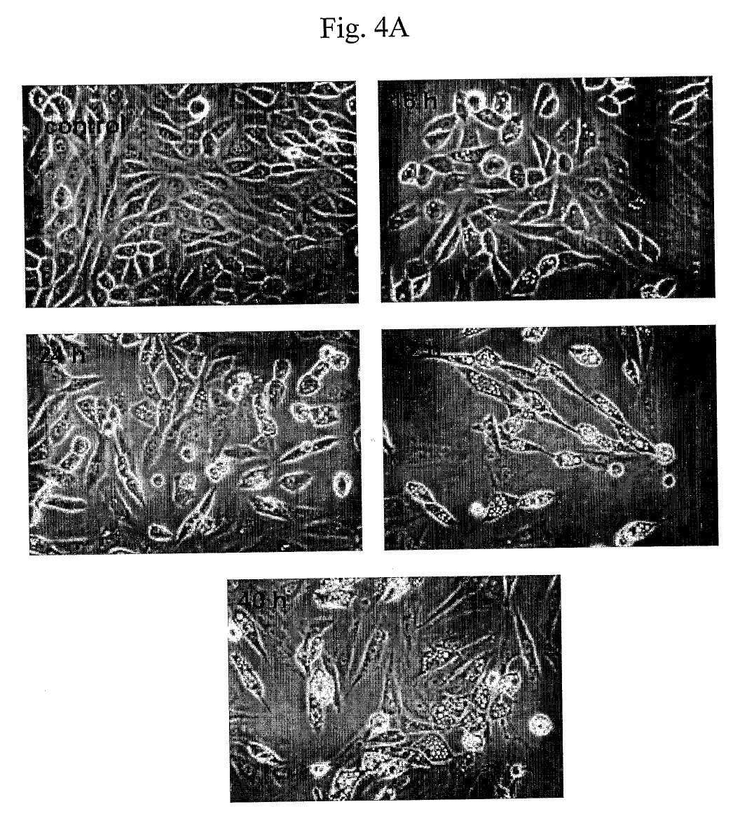

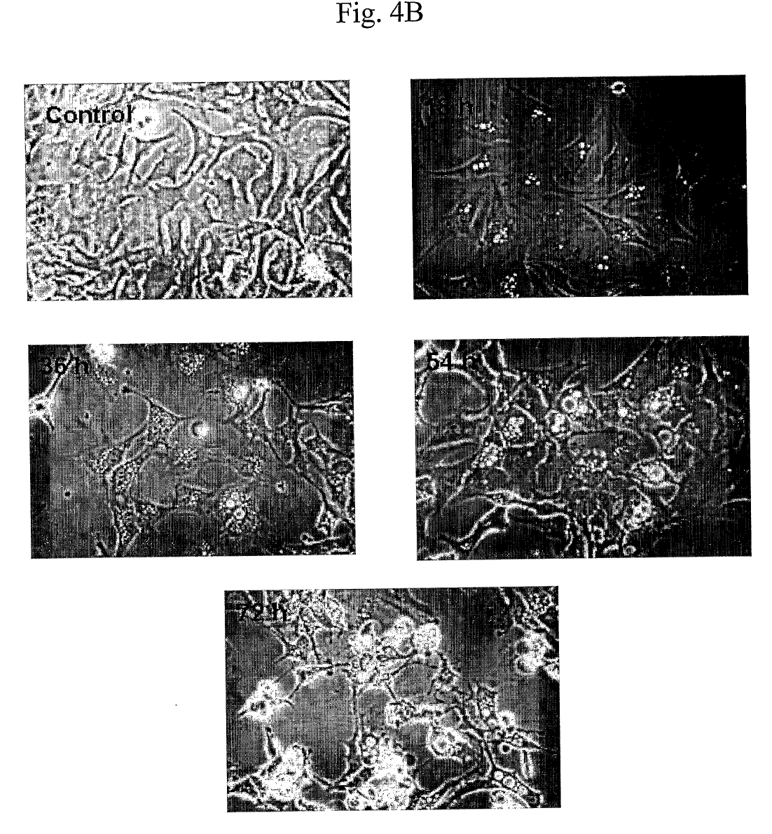

Mammalian cell monolayers were grown to 60-75% confluence in a CO₂ incubator at 37° C. Chinese hamster ovary (CHO) cells exposed to exogenous recombinant CARDS toxin displayed distinct vacuolization and cell rounding with disruption of monolayer integrity (FIG. 4A). Cytopathogenic effects (CPE) were slow to develop at low concentrations of CARDS toxin (10-50 ng/ml), requiring approximately 16-28 hours, while higher concentrations of CARDS toxin (10-50 μg/ml) elicited overt CPE in 4-12 hours. Heat inactivation of CARDS toxin preparations (30 minutes at 60° to 100° C.) abolished CPE.

To further examine the vacuolating effect of CARDS toxin on CHO cells, human immortalized HeLa cells were also exposed to CARDS toxin. As observed with CHO cell monolayers, intoxicated HeLa cells (FIG. 4B) displayed a highly vacuolated phenotype, which was dose and time dependent, and individual cells detached from the surface and exhibited cell pathology similar to that observed in the CARDS toxin-treated animal models described herein.

The characterization of this unusual ADP ribosylating and vacuolating toxin in M. pneumoniae provides insight into the wide-ranging pathogenic capabilities of M. pneumoniae and demonstrates the utility of the CARDS toxin as a diagnostic and prognostic indicator of infection and disease progression as well as a vaccine candidate and therapeutic toxin to control a variety of associated and implicated human pathologies.

### Effects of CARDS TX in Animals

Baboons: Individual animals were instilled with active toxin or non-toxin diluent into the right lower lobe by direct endoscopic placement. Baseline lavages were obtained from the contralateral lobes of each animal and follow-up bilateral lavages were obtained 24 and 48 hours after the initial inoculation to investigate systemic effects. Subsequently, animals were sacrificed and airways evaluated in both groups by histochemistry and by assessment of inflammatory responses in bronchoalveolar lavage (BAL).

In the CARDS toxin-treated animal, the trachea and right main and lower bronchi demonstrated a pronounced focal bronchiolar (small airway) ulceration and submucosal inflammation. Also, there was a striking bronchiolar infiltrate of lymphocytes and mononuclear cells and less pronounced presence of neutrophils. It was clear that an inflammatory response could be detected in both lower lobes, indicating a systemic effect of CARDS toxin. Bronchoalveolar lavages also revealed cellular infiltration and marked increases in inflammatory cytokines and chemokines were noted in intoxicated animals. For example, inflammatory cytokines, such as interleukin 6 (IL-6), increased 300 to 600 fold over the two-day period in the CARDS toxin-treated animal, indicating a powerful inflammatory and regulatory effect of CARDS toxin. Chemokines RANTES and IL-8 showed increases of 10 fold and 300 to 500 fold, respectively. No substantial stages in cytokine/chemokine profiles were observed in the negative control animal.

Mice: Individual animals were intranasally inoculated with CARDS toxin and monitored for changes in lung histopathology and BAL-associated inflammatory responses. Mice showed very striking peribronchiolar infiltration of lymphocytes, mononuclear cells and neutrophils. As in the case of baboons, marked lung injury was observed in the lungs and other airway tissues. The proliferation marker, MIB-1, was markedly reduced in CARDS toxin-treated mice, indicating a very powerful shut down of cell proliferation in contrast to non-toxin treated control animals. For example, IL-6 and IL-12 increased within the first six days of intranasal introduction of CARDS toxin by 8 fold and 20 fold, respectively. CARDS toxin can mimic the course of active mycoplasma infection both in terms of cytopathology and cytokine/chemokine responses.

These studies demonstrate that the CARDS toxin causes specific tissue and cell histopathology and cell death and elicits inflammatory cytokine/chemokine signature profiles in baboons and mice.

### Immunomodulating Activity of CARDS Toxin

Infection with Mycoplasma pneumoniae leads to the production of a variety of cytokines, chemokines and other immune signaling molecules characteristic of the infection. As noted above, application of the CARDS TX to mice or baboons induces essentially the same cytokine/chemokine response as infection with M. pneumoniae. This finding indicates that the CARDS TX is a key virulence factor responsible for triggering the characteristic immune response to M. pneumoniae. However, when the CARDS TX is considered as a singular entity (i.e., an individual chemical molecule) it is no longer a virulence factor but an immuno-modulator. That is, CARDS TX stimulates the immune system to induce a predictable immune response of cytokines and chemokines, etc., that is not being utilized for the survival of the pathogen. This has profound implications because many pathogens subvert the immune system by down-modulating, inhibiting, or misdirecting the cytokine, chemokine, and immune signaling responses designed to protect the host. Therefore, an immuno-modulator (i.e., CARDS TX) that is capable of inducing a specified immune response that a different pathogen needs to actively subvert in order to cause disease, could prevent disease caused by that pathogen.

A specific example of protection against plague (Yersinia pestis) pneumonia is described herein. The immune response induced by CARDS TX indicates that it could augment the innate protective immune response against many pathogens. Additionally, the response induced by this immuno-modulator suggests that it could be used as an adjuvant to enhance immunizations.

Plague pneumonia is a rapidly progressive pneumonia caused by Yersinia pestis that causes death in mice or humans in 2-4 days, depending on the susceptible species and infectious dose. Untreated, 100% of infected animals will die from the disease. In a mouse model, all mice are dead in 2-3 days from an infection with 500-800 bacteria given intranasally (IN). The yersinia bacterium possesses numerous virulence factors that actively modulate the host innate response, thereby allowing yersinia to survive and cause disease. One common feature is the early inhibition of inflammatory responses. Blocking these inflammatory cytokine/chemokine responses at the earliest points (i.e., first 24 hours of infection) is critical because, even though there is considerable cytokine expression on day 2 in Y. pestis-infected animals, they all die in 48-72 hours post infection (because of the active inhibition and delay in cytokine/chemokine profiles).

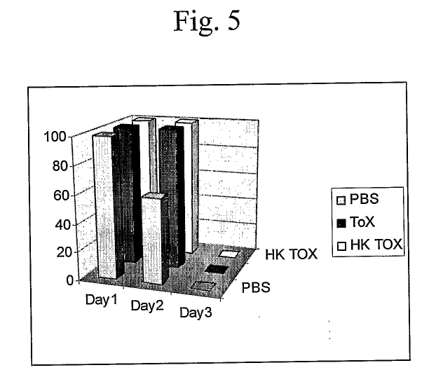

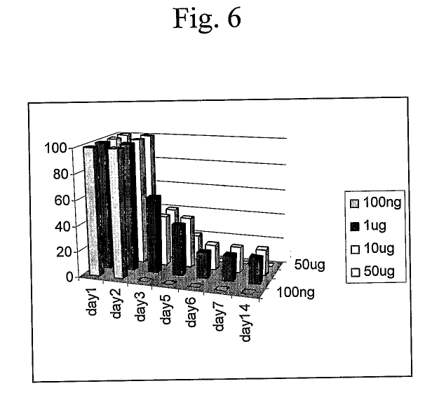

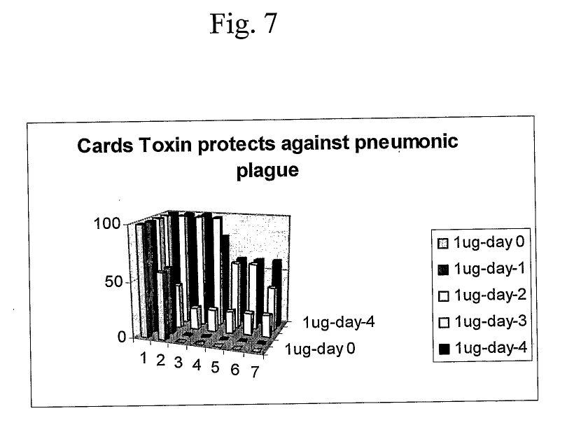

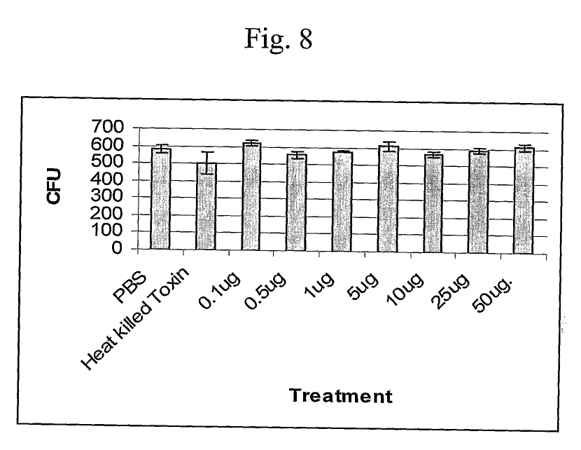

In the present example, CARDS TX administered intranasally (IN) induces a pro-inflammatory cytokine response in the bronchial alveolar lavage fluid (BALF) within days 1 and 2 of application to mice and baboons. Y. pestis initially induces an anti-inflammatory response at early time points (0-36 hours) and then a pro-inflammatory response >36 hrs post infection. Differential expression of cytokines continued in CARDS TX treated mice out to at least day 6. When mice were treated with various doses of CARDS TX intranasally (IN) and then challenged IN 4 days later with a lethal high or a lethal low dose of Yersinia pestis, improved mouse survival was observed, indicating that CARDS TX stimulated an immune response that reduced the degree of Y. pestis virulence capabilities. The dose of CARDS TX was optimized and 1 μg of toxin IN four days prior to infection gives the best improved survival in these studies. These results demonstrate that CARDS TX treatment significantly prolonged the average time to death in both treatment groups and completely prevented death in 20% of the animals challenged with the low lethal dose of Y. pestis (FIGS. 5 and 6).

As shown in Table 4, infection of the animals at the time of CARDS TX-intoxication or 1 day after intoxication provided no protection. 20% of the animals intoxicated two days prior to infection were protected. 80% of the animals survived if they were intoxicated 3-4 days prior to infection. 100% of the animals mock treated with saline or treated with 1 ug of heat killed toxin 4 days prior to infection died within two days of infection. Mice were challenged with 1000 cfu of Y. pestis strain CO92 IN. The IN LD-50 of CO92 is 50-120 bacteria. CARDS TX protection against infection is not due to a direct bactericidal effect on Y. pestis because growth of the bacteria in the presence of CARDS TX had no effect.

TABLE 4

| | | | Plague | Plague |
|---|---|---|---|---|
| Cytokine | Baboon day 2¹ | Mouse day 2² | day 1³ | day 2*** |
| IL-1beta | 0 | 0 | 0 | 600 |
| IL-6 | 3000 | 13 | 0 | 3000 |
| IL-10 | 0 | 4 | 0 | 0 |
| IL-12 | 4.5 | 7.4 | 0 | 0 |
| IFN-gamma | 2 | 3 | 0 | 0 |
| TNF-alpha | 0 | 10 | 0 | 50 |
| MCP-1 | 0 | 2 | 0 | 600 |
| KC/IL-8⁴ | 2000 | 15.5 | 0 | 1500 |

¹Cytokine levels in Baboon BALF reported as fold induction over time 0 levels
²Cytokine levels in mouse BALF reported as fold induction over time 0 levels
³Cytokine levels in mouse BALF after 1 or 2 days of plague infection reported as fold induction over time 0 levels
***These animals would have died within 12 hrs.

In summary, these studies show that recombinant CARDS TX protects against lethal challenge with Yersinia pestis. Greatest protection is provided by 1-10 μg of CARDS TX delivered intranasally 3-4 days prior to bacterial challenge. In different experiments, protection ranged from 20-60%. Toxin inactivated by heat or the toxin vehicle (phosphate buffered saline) provides no protection. In different experiments, protection ranged from 20-60% when animals were challenged with 10-20 LD-50s. In all incidences, recombinant toxin significantly delayed the time to death 30-100% depending on the challenge dose. CARDS TX induces an early pro-inflammatory response that may correlate with infection. The toxin is not bactericidal.

Chimeric Proteins with CARDS Toxin

In additional embodiments, the present invention provides a chimeric protein or polypeptide comprising, consisting essentially of and/or consisting of a CARDS toxin or biologically active fragment or domain thereof and a ligand for contacting the CARDS toxin or biologically active fragment or domain thereof with a target cell. Also provided herein is a nucleic acid molecule that encodes a chimeric protein of this invention, as well as a vector and/or cell comprising the nucleic acid molecule.

As described herein, a biologically active fragment of the CARDS toxin can be a fragment as described herein that retains one or more biological activities of the CARDS toxin, such as toxin activity. A biologically active fragment of this invention can also be a domain of the CARDS toxin, as described herein. The chimeric protein can comprise a “toxin domain,” which is a protein or functional fragment thereof that has toxic activity (e.g., ADP-ribosylating activity) and/or cytopathology inducing activity) on a cell as described herein. In some embodiments, in addition to a toxin domain of a CARDS toxin, the chimeric protein of this invention can comprise a toxin domain of another toxin, which can be, but is not limited to a toxin domain of diphtheria toxin, ricin, Pseudomonas exotoxin, colicin, anthrax toxin, tetanus toxin, botulinum toxin, saporin, abrin, bryodin, pokeweed anti-viral protein, viscumin and gelonin. A chimeric protein of this invention can comprise more than one (e.g., e, 3, 4, 5, or more) toxin domains or functional fragments thereof, which can be present in any order and/or in any combination in the chimeric protein. When multiple toxin domains are present, they can be immediately adjacent to one another, separated by one or more targeting moieties (antibody/ligand) and/or translocation domains, and/or separated by linkers. Furthermore, the moieties of the chimeric protein of this invention can be present in any order, multiplicity and/or combination relative to one another.

The chimeric proteins of this invention can also be modified by use in vivo by the addition of a blocking agent at the amino and/or carboxy-terminal end, to facilitate survival of the chimeric protein in vivo. Examples of blocking agents of this invention include, but are not limited to, additional related and/or unrelated peptide sequences that can be attached to either end of the chimeric protein. Blocking can be carried out either chemically during synthesis of the chimeric protein of by recombinant DNA technology according to protocols well known in the art.

The ligand moiety of the chimeric protein of this invention can be an antibody that specifically reacts with an antigen on a cell surface, such that the antibody will bind to the surface of a cell possessing the antigen (a target cell), thereby bringing the CARDS toxin moiety of the chimeric protein in contact with the cell. The CARDS toxin moiety can be internalized by the target cell and the CARDS toxin or biologically active fragment thereof is active in the target cell, resulting in damage to and/or death of the target cell.

The ligand moiety of the chimeric protein can be an antibody to a cancer antigen, which can be an antigen that is present only on the surface of a cancer cell and/or it can be a cancer-associated antigen that is present on the surface of a cancer cell in an amount greater than the amount of antigen that would be present on the surface of a non-cancerous (e.g., normal) cell.

A cancer antigen of this invention can include, but is not limited to HER2/neu and BRCA1 antigens for breast cancer, MART-1/MelanA, gp100, tyrosinase, TRP-1, TRP-2, NY-ESO-1, CDK-4, β-catenin, MUM-1, Caspase-8, KIAA0205, HPVE7, SART-1, PRAME, and p15 antigens, members of the MAGE family, the BAGE family (such as BAGE-1), the DAGE/PRAME family (such as DAGE-1), the GAGE family, the RAGE family (such as RAGE-1), the SMAGE family, NAG, TAG-72, CA125, mutated proto-oncogenes such as p21ras, mutated tumor suppressor genes such as p53, tumor associated viral antigens (e.g., HPV16 E7), the SSX family, HOM-MEL-55, NY-COL-2, HOM-HD-397, HOM-RCC-1.14, HOM-HD-21, HOM-NSCLC-11, HOM-MEL-2.4, HOM-TES-11, RCC-3.1.3, NY-ESO-1, and the SCP family. Members of the MAGE family include, but are not limited to, MAGE-1, MAGE-2, MAGE-3, MAGE-4 and MAGE-11. Members of the GAGE family include, but are not limited to, GAGE-1, GAGE-6. See, e.g., review by Van den Eynde and van der Bruggen (1997) in Curr. Opin. Immunol. 9: 684-693, Sahin et al. (1997) in Curr. Opin. Immunol. 9: 709-716, and Shawler et al. (1997), the entire contents of which are incorporated by reference herein for their teachings of cancer antigens.

The cancer antigen can also be, but is not limited to, human epithelial cell mucin (Muc-1; a 20 amino acid core repeat for Muc-1 glycoprotein, present on breast cancer cells and pancreatic cancer cells), MUC-2, MUC-3, MUC-18, the Ha-ras oncogene product, carcino-embryonic antigen (CEA), the raf oncogene product, CA-125, GD2, GD3, GM2, TF, sTn, gp75, EBV-LMP 1 & 2, HPV-F4, 6, 7, prostatic serum antigen (PSA), prostate-specific membrane antigen (PSMA), prostate stem cell antigen (PSCA), alpha-fetoprotein (AFP), C017-1A, GA733, gp72, p53, the ras oncogene product, β-HCG, gp43, HSP-70, p17 mel, HSP-70, gp43, HMW, HOJ-1, melanoma gangliosides, TAG-72, mutated proto-oncogenes such as p21ras, mutated tumor suppressor genes such as p53, estrogen receptor, milk fat globulin, telomerases, nuclear matrix proteins, prostatic acid phosphatase, protein MZ2-E, polymorphic epithelial mucin (PEM), folate-binding-protein LK26, truncated epidermal growth factor receptor (EGFR), Thomsen-Friedenreich (T) antigen, GM-2 and GD-2 gangliosides, polymorphic epithelial mucin, folate-binding protein LK26, human chorionic gonadotropin (HCG), pancreatic oncofetal antigen, cancer antigens 15-3, 19-9, 549, 195, squamous cell carcinoma antigen (SCCA), ovarian cancer antigen (OCA), pancreas cancer associated antigen (PaA), mutant K-ras proteins, mutant p53, chimeric protein p210_{BCR-ABL}, urokinase-type plasminogen activator receptor (uPAR), tumor associated viral antigens (e.g., HPV16 E7) and SP-A (see, e.g., Khubchandani and Snyder. “Surfactant Protein A (SP-A): the alveolus and beyond” FASEB J. 15:59-69 (2001).

The cancer antigen of this invention can also be an antibody produced by a B cell tumor (e.g., B cell lymphoma; B cell leukemia; myeloma; hairy cell leukemia), a fragment of such an antibody, which contains an epitope of the idiotype of the antibody, a malignant B cell antigen receptor, a malignant B cell immunoglobulin idiotype, a variable region of an immunoglobulin, a hypervariable region or complementarity determining region (CDR) of a variable region of an immunoglobulin, a malignant T cell receptor (TCR), a variable region of a TCR and/or a hypervariable region of a TCR. In one embodiment, the cancer antigen of this invention can be a single chain antibody (scFv), comprising linked V_{H}, and V_{L} domains, which retains the conformation and specific binding activity of the native idiotype of the antibody.

The present invention is in no way limited to the cancer antigens listed herein. Other cancer antigens be identified, isolated and cloned by methods known in the art such as those disclosed in U.S. Pat. No. 4,514,506, the entire contents of which are incorporated by reference herein.

The present invention further provides a method for treating cancer, a method for killing a tumor cell and/or a method for reducing the size of a tumor in a subject, comprising administering to the subject and/or contacting cancer cells (e.g., tumor cells) of the subject with a chimeric protein of this invention and/or a nucleic acid encoding a chimeric polypeptide of this invention.

The cancer to be treated by administration to a subject of a chimeric polypeptide and/or nucleic acid encoding a chimeric polypeptide of this invention can be, but is not limited to, B cell lymphoma, T cell lymphoma, myeloma, leukemia, hematopoietic neoplasia, thymoma, lymphoma, sarcoma, lung cancer, liver cancer, non-Hodgkins lymphoma, Hodgkins lymphoma, adrenal cancer, anal cancer, colorectal cancer, endometrial cancer, esophygeal cancer, fallopian tube cancer, gallbladder cancer, gastric cancer, glioblastoma, kidney cancer, laryngeal cancer, medulloblastoma, mesothelioma, neuroblastoma, oropharyngeal cancer, osteosarcoma, parathyroid cancer, thyroid cancer, penile cancer, pituitary cancer, retinoblastoma, rhabdomyosarcoma, urethral cancer, uterine cancer, adenocarcinoma, breast cancer, pancreatic cancer, colon cancer, lung cancer, renal cancer, bladder cancer, liver cancer, prostate cancer, ovarian cancer, primary or metastatic melanoma, squamous cell carcinoma, basal cell carcinoma, brain cancer, angiosarcoma, hemangiosarcoma, head and neck carcinoma, thyroid carcinoma, soft tissue sarcoma, testicular cancer, uterine cancer, cervical cancer, vaginal cancer, vulvar cancer, small intestinal cancer, and any other cancer now known or later identified (see, e.g., Rosenberg (1996) Ann. Rev. Med. 47:481-491, the entire contents of which are incorporated by reference herein).

In other embodiments, the ligand of this invention can be a ligand that specifically binds a receptor on a muscle cell, which can include, but is not limited to, an antibody reactive to N-CAM (neuronal cell adhesion molecule) (Sigma Chemical Company, St. Louis, Mo.), an antibody reactive with the muscle-specific antigen, Leu-19, an antibody reactive with dystrophin (Sigma) and an antibody reactive with a nicotinic acetylcholine receptor (nAchR). The nAch receptor and antibodies generated thereto are readily available from publicly accessible depositories. (See, e.g., U.S. Pat. No. 5,192,684, ATCC Nos.: HB 8987 (mAb 64), HB 189 (mAb 270), and TIB 175 (mAb 35), all of which are incorporated herein by reference.)

Thus, the present invention also provides methods of treating a muscle spasm, (e.g., a focal muscle spasm; a facial wrinkle), comprising contacting a muscle cell of the muscle in spasm with a chimeric protein of this invention. Such methods can be used, for example, to treat blepharospasm, cervical dystonia, hand dystonia, limb dystonia, hemifacial spasm, bruxism, strabismus, VI nerve palsy, spasmodic dysphonia and/or oromandibular dystonia, as well as any other disease or disorder associated with muscle spasm that is now known or later identified. A method of treating a muscle spasm according to this invention can include, for example, administering (e.g., by intramuscular injection) an effective amount of the chimeric polypeptide and/or nucleic acid encoding the chimeric polypeptide of this invention to the muscle of a subject. In some embodiments, the chimeric polypeptide and/or nucleic acid of this invention can also be administered with an effective amount of botulinum toxin, either alone and/or as part of a chimeric polypeptide and/or encoded by a nucleic acid molecule.

As used herein, a “muscle spasm” includes a brief, unsustained contraction and/or a paroxysmal, spontaneous, prolonged contraction of one or more muscles, which are responsive to treatment involving selective destruction of one or more muscles at the site of the muscle spasm.

The production of any type of antibody and/or antibody fragment, (e.g., monoclonal, polyclonal, humanized, human, single chain, single chain Fv, Fab, F(ab′)₂, Fc, bispecific, multispecific, etc.) can be carried out according to well developed in the art. A humanized or “chimeric” antibody is an immunoglobulin molecule comprising a human moiety and a non-human moiety, in any combination. For example, the variable region of a humanized immunoglobulin molecule can be from a non-human (e.g., murine) source and the constant region can be from a human source. Thus, in this example, the humanized antibody can have the antigen-binding specificity of the non-human source and the effector function of the human source (see, e.g. U.S. Pat. No. 5,482,856, the entire contents of which are incorporated by reference herein). Protocols for the production of human antibodies are well known (see, e.g., U.S. Pat. No. 5,001,065, the entire contents of which are incorporated by reference herein).

In certain embodiments, the chimeric protein of this invention can comprise a translocation or internalization domain of a toxin protein, e.g., to facilitate delivery of the toxin moiety into the target cell. The translocation domain can be from the CARDS toxin described herein and/or the domain can be from any other toxin that has a translocation domain. Such toxins include but are not limited to diphtheria toxin, colicin, delta-endotoxin, anthrax toxin, tetanus toxin, botulinum toxin and Pseudomonas exotoxin.

The chimeric proteins of this invention are produced by methods well known in the art. For example, an antibody or ligand that allows for specific targeting of the CARDS toxin to a specific cell type or population can be selected according to art-known procedures for the specific therapeutic effect desired. The antibody or ligand can be joined via a covalent or non-covalent bond to a CARDS toxin and/or biologically active fragment thereof as described herein. The moieties can be attached, joined or connected by any of a number of means well known to those of skill in the art. In some embodiments, the chimeric protein of the present invention can be recombinantly expressed as a single-chain fusion protein comprising both antibody and toxin according to methods well known in the art. The toxin moiety can be joined, linked or conjugated directly, or through a linker (spacer), to the ligand.

A “linker” as used herein, is a molecule that is used to join two molecules. The linker is capable of forming covalent bonds or high-affinity non-covalent bonds to both molecules. Suitable linkers are well known to those of ordinary skill in the art and include, but are not limited to, straight or branched-chain carbon linkers, heterocyclic carbon linkers and/or peptide linkers. The linkers can be joined to the constituent amino acids through their side groups (e.g., through a disulfide linkage to cysteine).

The procedure for attaching a toxin to an antibody or other polypeptide targeting molecule can vary according to the chemical structure of the toxin. Immunoglobulin molecules contain a variety of functional groups; e.g., sulfhydryl (—S), carboxylic acid (COOH) and free amine (NH₂) groups, which are available for reaction with a suitable functional group on a toxin. Additionally or alternatively, the antibody/ligand and/or toxin can be derivatized to expose or attach additional reactive functional groups. The derivatization can involve attachment of any of a number of linker molecules (including commercially available linker molecules, e.g., from Pierce Chemical Company, Rockford Ill.).

In some embodiments, a bifunctional linker having one functional group reactive with a group on the toxin and another functional group reactive with a group on an antibody/ligand, can be used. Derivatization can also involve chemical treatment of the toxin or antibody/ligand (e.g., by glycol cleavage of the sugar moiety of a glycoprotein antibody with periodate to generate free aldehyde groups). In some embodiments, the free aldehyde groups on an antibody can be reacted with free amine or hydrazine groups on a toxin to form the chimeric protein (see, e.g., U.S. Pat. No. 4,671,958. the entire contents of which are incorporated by reference herein). Procedures for generation of free sulfhydryl groups on an antibody or antibody fragment, are also known (see, e.g., U.S. Pat. No. 4,659,839).

Many procedures and linker molecules for attachment of various compounds including toxins are known. See, for example, European Patent Application No. 188,256; U.S. Pat. Nos. 4,671,958, 4,659,839, 4,414,148, 4,699,784; 4,680,338; 4,569,789; 4,589,071; and Borlinghaus et al. Cancer Res. 47: 4071-4075 (1987), which are incorporated herein by reference in their entireties. Production of various chimeric proteins for use as immunotoxins is also well-known (Thorpe et al. “Monoclonal Antibody-Toxin Conjugates: Aiming the Magic Bullet” Monoclonal Antibodies in Clinical Medicine Academic Press, pp. 168-190 (1982); Waldmann. Science 252:1657 (1991); U.S. Pat. Nos. 4,545,985 and 4,894,443, the entire contents of each of which are incorporated herein by reference. See also, e.g., Birch and Lennox. Monoclonal Antibodies: Principles and Applications Chapter 4, Wiley-Liss, New York, N.Y. (1995); U.S. Pat. Nos. 5,218,112, 5,090,914). In some embodiments, the linker molecule is m-Malimidobenzoyl-N-hydroxysuccinimideester (MBS) which can be used to prepare chimeric proteins as described, for example, in Youle and Nevelle. Proc. Natl. Acad. Sci. 77(9):5483-5486 (1980).

In some embodiments, it may be desirable to free the toxin from the antibody when the chimeric protein has reached its target site. Therefore, the present invention further provides a chimeric protein comprising a linkage that is cleavable in the vicinity of or within the target site and that can be used when the toxin is to be released at the target site. Cleavage of the linkage to release the toxin from the antibody/ligand can be facilitated by enzymatic activity and/or conditions to which the immunoconjugate is subjected either inside the target cell or in the vicinity of the target site. Several different cleavable linkers are known in the art (see, e.g., U.S. Pat. Nos. 4,618,492; 4,542,225, and 4,625,014, the entire contents of which are incorporated by reference herein). As one example, SPDP is a reversible NHS-ester, pyridyl disulfide cross-linker used to conjugate amine-containing molecules to sulfhydryls. Another chemical modification reagent is 2-iminothiolane, which reacts with amines and yields a sulfhydryl. Water soluble SPDP analogs, such as Sulfo-LC-SPDP (Pierce, Rockford, Ill.) can also be used. As another example, SMPT is a reversible NHS-ester, pyridyl disulfide cross-linker developed to avoid cleavage in vivo prior to reaching the target cell or site.

The chimeric protein of this invention can also be produced according to standard protocols for recombinant DNA technology. The chimeric proteins can be produced in any number of well-defined expression systems and purified according to act-known standards for in vivo administration. Alternatively, a nucleic acid encoding the chimeric protein can be administered to a subject and/or to a cell of a subject of this invention and the chimeric protein can be produced in the cell or in the subject. Thus, the chimeric proteins and nucleic acids encoding them can be administered in vivo or ex vivo.

The chimeric protein of this invention can be present in a composition, which can be a pharmaceutical composition comprising the chimeric protein and/or nucleic acid molecule encoding a chimeric protein in a pharmaceutically acceptable carrier. As described herein, “pharmaceutically acceptable” includes a material that is not biologically or otherwise undesirable, i.e., the material can be administered to an individual along with the chimeric polypeptide and/or nucleic acid without causing substantial deleterious biological effects or interacting in a deleterious manner in the subject and/or with any of the other components of the composition in which it is contained. Methods for preparing pharmaceutically acceptable compositions are well known in the art and are described in more detail in such publications as Remington's Pharmaceutical Science, 19th ed., Mack Publishing Company, Easton, Pa. (1995).

As will be readily apparent to any clinician of ordinary skill in the art, the dose of the chimeric protein and/or nucleic acid encoding the chimeric protein will depend upon the properties of the particular chimeric polypeptide employed, e.g., its activity and biological half-life, the concentration of chimeric polypeptide in the formulation, the site and rate of administration, the clinical tolerance and characteristics (e.g., sex, gender, species, age, size, weight, overall condition) of the patient involved, the nature and severity of the disease or disorder to be treated or altered, etc., as are well know considerations in the art.

It is well understood that the chimeric protein and/or nucleic acid encoding the chimeric protein of this invention can be administered to a subject in a variety of ways (e.g., orally, intravenously, subcutaneously, intramuscularly, intratumorally, intraperitoneally, intrarectally, intravaginally, intranasally, intragastrically, intratracheally and/or intrapulmonarily.

In some embodiments, the chimeric polypeptide of this invention can be administered to a subject by, e.g., injection, into a muscle or into a tumor, in an amount ranging from about 1 ng to about 500 mg. An effective amount can be determined by one of skill in the art, using art-known teachings such as those provided in MacDonald and Glover (“Effective tumor targeting: Strategies for delivery of armed antibodies” Current Opinion in Drug Discovery and Development 8:177-183 (2005) and Michl and Gress (“Bacteria and bacterial toxins as therapeutic agents for solid tumors” Current Cancer Drug Targets 4:689-702 (2004). When treating muscle spasms, the chimeric protein can be administered at the site of the neuromuscular junctions of the muscle to be treated. As noted above, in various embodiments, the chimeric proteins of the present invention can be administered alone, in combination, and/or in conjunction with a conjugated and/or an unconjugated form of a different toxin (e.g., botulinum toxin).

The chimeric protein and/or nucleic acid can be administered once or it can be administered periodically until either a therapeutic result is achieved or until side effects warrant discontinuation of therapy. Generally, the dose should be sufficient to treat or ameliorate known symptoms or signs (e.g., release of muscle spasm; decrease in tumor size or reduction in tumor cell count) without producing unacceptable toxicity to the subject. An effective amount is an amount that provides either subjective relief of at least one symptom and/or an objectively identifiable improvement as noted by the clinician or other qualified observer.

The chimeric protein and/or nucleic acid can also be administered via microspheres, liposomes or other microparticulate delivery systems placed in certain tissues, including blood.

Testing of any of the chimeric proteins and nucleic acids encoding chimeric proteins of this invention for cytotoxicity both in vitro and in vivo can be carried out according to protocols well described in the art. For example, methods of determining cytotoxicity of a chimeric protein of this invention used to treat muscle spasms are described in U.S. Pat. No. 6,780,413, the entire contents of which are incorporated by reference herein. Methods of determining the cytotoxicity of a chimeric protein of this invention used to treat cancer and/or to kill tumor cells are described in U.S. Pat. No. 6,846,484, the entire contents of which are incorporated by reference herein.

Mycoplasma penetrans Toxin

The present invention provides a protein of M. penetrans that is shown herein to have activities similar to the CARDS toxin. To identify this protein, sequence of M. penetrans proteins were examined for a motif found in ADP-ribosylating bacterial toxins, which is the presence of two essential amino acid residues, which are arginine at position 28 and glutamic acid at position 156. The protein MYPE9110 was identified as having this motif. In order to express a full length protein in E. coli BL21 (DE3) LpxM, all 7 UGAs encoding tryptophan in MYPE9110 were changed to UGG. The recombinant protein was then purified using nickel column chromatography.

Using the established ADP-ribosylation assay described herein for the CARDS TX, the recombinant MYPE9110 protein was evaluated for ADP-ribosylating activity. CHO cell lysates were incubated with recombinant MYPE9110 in 1 mM ATP, 1 mM GTP, 10 mM thymidine, 10 mM DTT, 50 mM Tris (pH 7.4) with 5% glycerol and 10 μM ³²P labeled NAD (10 Ci/mmol). The reaction volume was incubated at 37° C. for 30 minutes, followed by the addition of 20% TCA to precipitate proteins. This mixture was then spun at 13,000 g for 10 minutes. The supernatant was removed, and the cell pellet containing the ADP-ribosylated host cell target proteins was dissolved in SDS-PAGE sample buffer (Nu-Page) and heated for 70° C. for 10 minutes. These samples were loaded into 10% SDS PAGE gels (Nu-Page) and run for 60 minutes at 200V. These gels were transferred to nitrocellulose membranes for 1 hour at 15V. Recombinant M. penetrans toxin was shown to ADP ribosylate specific mammalian proteins (˜19 kDa, 33 kDa and 35 kDa) and demonstrated auto ADP ribosylation (i.e., ADP ribosylates ‘self’ at 68 kDa).

The effect of recombinant M. penetrans toxin on monolayer cultures of HeLa cells grown in 25 cm² flasks in MEM with 10% serum at 37C and 5% CO2 was also evaluated. Recombinant MYPE9110 (2 μg, 10 μg, and 30 μg/ml) was added to these cells and incubated at 37° C. for 3 hours in 5% CO₂. 5 mM ammonium chloride was added to the HeLa cells infected with recombinant M. penetrans toxin at 3 hours and again incubated at 37° C. in 5% CO₂. Pronounced vacuolization was observed at all concentrations in HeLa cells while the PBS control and heat inactivated MYPE9110 protein treated cells showed no cytotoxicity.

These studies identify MYPE9110 as another member of the ADP-ribosylating, vacuolating toxins unique to mycoplasmas.

### Role of Mycoplasma Toxins in Airway Disorders and Asthma

Certain aspects of this invention are based on the identification of the CARDS TX as an important mediator of asthma, thus allowing for the development of effective strategies to diagnose, treat and prevent asthma and related airway diseases in a substantial population of both children and adults.

Asthma is a complex disease that afflicts over 15 million Americans. Despite the apparent increase in prevalence of disease within our population, asthma is still a poorly understood disease. This is in part due to the complex mixture of genetic factors, environmental stimuli, and immune system status that impacts disease development and progression.

Asthma is a complex disease since it involves genetic predisposition, environmental factors, and an interaction with the immune status in the development and progression of the disease. Over 15 million Americans are afflicted with this disease and despite the use of potent medications, between 16-17% of patients experience continuous daily and frequent nocturnal symptoms. One underappreciated and controversial factor in the etiology of asthma is the role that atypical bacterial infections, such as those caused by Mycoplasma pneumoniae, play in initiating, exacerbating and prolonging airway-related symptoms and pathologies.

Multiple lines of evidence directly link M. pneumoniae to the pathogenesis of asthma beyond its role as a precipitating factor in acute exacerbation of asthma. In children, M pneumoniae infections have been shown to induce chronic lung damage for prolonged periods after the resolution of respiratory tract symptoms. Studies have demonstrated abnormal pulmonary function tests in up to 50% of children and abnormalities of the lung in 37% of children months to years after an episode of M. pneumoniae respiratory infection. Mycoplasma pneumoniae is also known to induce a number of inflammatory mediators implicated in the pathogenesis of asthma. IgE, IL-4, and IL-5 have been shown to be significantly elevated in children with M. pneumoniae infections, suggesting that M. pneumoniae can induce a Th2 like cytokine response. In adults, M. pneumoniae has been detected in a large percentage of patients with stable moderately severe chronic asthma. The significance of this finding was supported by a randomized, double-blind study that demonstrated only PCR positive asthmatics improved their pulmonary function test when treated with antibiotic therapy directed against mycoplasmas.

Although M. pneumoniae infection has been suspected to be associated with the onset, exacerbation, and chronicity of asthma, no single M. pneumoniae virulence determinant or other mycoplasma molecule has been previously identified as a mediator of asthma-related symptoms, associated pathologies, or acute and chronic airway inflammation. This lack of definable M. pneumoniae pathogenic factors has prevented the establishment of cause-and-effect linkages between M. pneumoniae, asthma and related airway diseases. It also has prevented the development of effective interventions to control or prevent asthma pathogenesis. The CARDS TX of this invention, which is the first ADP-ribosylating (and vacuolating) toxin associated with any pathogenic human or animal mycoplasma, represents the first bona fide virulence factor identified that provides an association between M. pneumoniae infection and acute and chronic airway disease and extra-pulmonary manifestations.

Project 1. Mycoplasma pneumoniae rCARDS TX as Mediator of Airway Dysfunction in Mice

The present invention provides evidence for a role of the CARDS TX in the pathogenesis associated with airway inflammation, airway obstruction and airway hyperreactivity associated with respiratory M. pneumoniae infection. Additional studies are being conducted to identify the role played by the CARDS toxin in the ability of M. pneumoniae to induce acute asthma exacerbations and in the deleterious long-term effects of mycoplasma respiratory tract infection. Therapeutic interventions directed against CARDS TX can then be developed to treat and/or prevent M. pneumoniae-associated reactive airway disease and asthma. The specific aims of these studies are to 1) understand the specific contribution of active CARDS TX to the airway obstruction, hyperreactivity, and inflammation observed in M. pneumoniae respiratory infection; 2) determine if the host immune response to CARDS TX is protective against the respiratory manifestations of M. pneumoniae infection; and 3) determine the effect of bacterial protein synthesis inhibitor therapy on CARDS TX protein production in M. pneumoniae respiratory infection. The goal of these investigations is to develop disease modifying strategies to treat children and adults with mycoplasma-associated reactive airway disease and asthma. This project focuses on testing the CARDS TX, in an established acute and chronic murine model of M. pneumoniae respiratory infection in which airway inflammation, airway obstruction, and airway hyperreactivity have been previously characterized. BALB/c mice will be exposed to M. pneumoniae (wild-type and CARDS TX null mutant) or recombinant CARDS TX to determine the contribution of CARDS TX to the airway manifestations of M. pneumoniae infection. In addition, therapeutic interventions directed against CARDS TX will be assessed in the murine model with the goal of translational applicability to the treatment of reactive airway disease and asthma associated with M. pneumoniae in children and adults.

Investigations of CARDS TX in a Murine Model of Acute and Chronic M. pneumoniae Infection

Active CARDS TX induces histologic pulmonary inflammation in mice (and baboons) consistent with M. pneumoniae infection, while heat-inactivated toxin does not. BALB/c mice were inoculated intranasally once with either active recombinant CARDS TX, heat-inactivated rCARDS TX, or the toxin carrier solution (controls). Mouse lungs were examined at 2, 4, and 6 days post-inoculation. Lungs were removed, fixed in 10% neutral buffered formalin, embedded in paraffin, sectioned, and H&E stained. The lungs from mice given CARDS TX demonstrated acute histologic inflammation similar to that seen with M. pneumoniae infection in BALB/c mice. Peribronchial and perivascular mononuclear infiltrates, predominately lymphocytic, were present in all CARDS TX treated mice. Lungs from mice inoculated with heat-inactivated toxin demonstrated minimal inflammation, and lungs from control mice inoculated with the toxin carrier solution had no histologic abnormalities. Based on the histologic scoring system developed by Cimolai et al. (1992) for experimental M. pneumoniae infection (the greater the score the greater the inflammation), the CARDS TX treated lungs had significantly greater inflammation than the control mice.

A similar experiment was performed in two adult baboons; the baboons were given either active rCARDS TX or heat-inactivated rCARDS TX by bronchoscope. The baboon lung findings paralleled the mouse lung findings, with the baboon lung inoculated with heat-inactivated rCARDS TX demonstrating minimal inflammatory infiltrate and the baboon lung inoculated with active rCARDS TX demonstrating peribronchiolar lymphocytic infiltrate and sloughed epithelium. These animal model findings identify CARDS TX as an important virulence factor in M. pneumoniae respiratory infection. In addition, the inflammatory activity of this protein appears to be due to its active toxin properties.

### Active CARDS TX Induces the Production of Cytokines/Chemokines in the Lower Respiratory Tract of Mice and Baboons.

The concentrations of cytokines and chemokines in bronchoalveolar lavage fluid (BAL) from BALB/c mice after intranasal inoculation with active rCARDS TX or control carrier solution (or heat-inactivated toxin) were evaluated. Two days after inoculation, BAL was collected and the concentrations of cytokines and chemokines were determined using multiplex bead immunoassays with Luminex LabMAP system. We examined the levels of IL-1β, IL-2, IL-4, IL-5, IL-6, IL-10, IL-12, TNF-α, IFN-α, GM-CSF, KC, IP-10, MIP-1α, MIG, and MCP-1. Statistically significant elevations in cytokine/chemokine concentrations for active CARDS TX compared with controls were observed for TNF-α, GM-CSF, IL-1β, IL-6, IL-12, KC and MIP-1α). The other cytokine/chemokines tested did not show significant differences. This observed BAL cytokine/chemokine profile is consistent with the profile observed in applicants' mouse model of M. pneumoniae infection. This profile is most compatible with a proinflammatory and Th1 response to CARDS TX.

In a parallel experiment, two adult baboons were given either active rCARDS TX or heat-inactivated rCARDS TX by bronchoscope. Significant elevations in BAL cytokines and chemokines were found in the active rCARDS TX treated baboon compared with the baboon that received heat-inactivated rCARDS TX as follows: G-CSF (40 fold), IL-1Ra (10 fold), IFN-α (75 fold), IL-6 (100 fold), IL-8 (600 fold), MIP-1α (5 fold), and RANTES (9 fold).

Active rCARDS TX Induces Airway Obstruction and Airway Hyperreactivity.

To assess the effect of active rCARDS TX on respiratory function, mice were inoculated with rCARDS TX as described herein and airway obstruction and airway hyperreactivity (AHR) were measured before and after aerosolized methacholine, respectively, by plethysmography (enhanced pause) in nonsedated, unrestrained mice. Enhanced Pause (Penh) is a dimensionless value that represents a function of the ratio of peak expiratory flow to peak inspiratory flow and a function of the timing of expiration. Penh does not directly measure airway resistance. Adler and colleagues (2004) have recently re-confirmed that in BALB/c mice Penh strongly and significantly correlates with lung resistance measured under anesthesia while animals were mechanically ventilated; this was demonstrated by comparing unrestrained plethysmography and invasive monitoring in the same mice. In addition, Penh as measured by unrestrained plethysmography has been validated in multiple animal models of airway obstruction and airway hyperresponsiveness (Ackerman et al, 2005; Gonzalo et al, 1998; Hamelmann et al, 1997; Krane et al, 2001; Schwarze et al, 1997; van Schaik, 1998).

The results of these studies demonstrate that active CARDS TX induced significant elevations in airway obstruction and airway hyperreactivity compared with the toxin carrier solution. These data indicate that active CARDS TX may mediate the ability of M. pneumoniae to induce acute asthma exacerbations and may be responsible for the deleterious long-term effects of mycoplasma respiratory tract infection.

The anti-M. pneumoniae Antibody Response in the Acute and Chronic Stages of Murine M. pneumoniae Respiratory Tract Infection Appears Protective.

Serum anti-M. pneumoniae IgM and IgG titers have been investigated in the acute and chronic stages of murine M. pneumoniae respiratory tract infection (Hardy et al, 2001; Hardy et al, 2002). Significant inverse correlations between serum anti-M. pneumoniae IgG titers and markers of disease severity have been documented that suggest a protective role of the antibody response in both the acute and chronic stages of infection. For example, a significant inverse correlation was found between serum anti-M. pneumoniae IgG titers and both M. pneumoniae BAL quantitative cultures (r=−0.68, p<0.001) and airway obstruction (r=−0.81, p=0.01) in the acute stage of disease.

In chronic disease in the applicants' model, when serum IgG titers against M. pneumoniae were waning and inflammatory lung histopathology was present, the concentrations of IgG antibodies significantly inversely correlated with the severity scoring of the inflammatory lung histopathology (r=−0.95, p=0.01) (the higher the anti-M. pneunoniae IgG titer, the lower the pulmonary cellular histologic inflammation, and vice versa). One possible explanation for this finding is that as anti-M. pneumoniae IgG titers fall in chronic disease, M. pneumoniae becomes able to exert a pathogenic effect that is otherwise inhibited by high anti-M. pneumoniae IgG titers. These findings indicate a protective role of the host response against mechanisms responsible for abnormal pulmonary function and histologic inflammation in the acute and chronic stages of mycoplasma respiratory disease.

Mice Infected with M. pneumoniae Develop a Specific Antibody Response to CARDS TX.

Mice inoculated with M. pneumoniae to produce respiratory tract infection have been shown to develop specific anti-CARDS TX serum IgG antibody. Studies will be carried out to determine if antibodies to the CARDS TX are protective, thus leading to the development of vaccines or to immune-based therapies specific for acute and/or chronic M. pneumoniae respiratory infection associated with reactive airway disease or asthma.

Patients who fully recover from naturally acquired or experimentally induced M. pneumoniae respiratory disease are generally resistant to reinfection or rechallenge. Previous immunization strategies against M. pneumoniae have only offered partial protection and are thought to have been hampered by a lack of knowledge of the basic virulence mechanism responsible for disease (Barile, 1984). The CARDS TX of the present invention presents new opportunities for immunization and treatment strategies against M. pneumoniae.

In studies with antimicrobials, regardless of class (macrolide, ketolide, or peptide deformylase inhibitor), varying degrees of disassociation have been observed between the antimicrobial and the anti-inflammatory effects of these bacterial protein synthesis inhibitors. The greatest disassociation in this regard has been observed for clarithromycin and LBM 415 (NVP PDF-713). When mice were studied approximately one week after antimicrobial therapy was stopped, there was no difference in the number of M. pneumoniae organisms recovered from BAL between the antimicrobial and placebo therapy groups. However, significant differences did exist in the inflammatory histopathologic score between the antimicrobial and placebo therapy groups; therefore, no microbiologic difference was noted, while tissue inflammation was significantly reduced after either therapy. Significant improvement in inflammatory histopathologic score and airway obstruction at days 1 and 3 of therapy was observed before a significant reduction in M. pneumoniae organisms was noted at day 6 of therapy; in other words, no microbiologic difference was noted at days 1 and 3 of therapy, while tissue inflammation and airway obstruction were significantly reduced at days 1 and 3. These data indicate that these therapies are having an effect on tissue inflammation and airway function to a much greater degree than on the number of viable M. pneumoniae recovered after treatment.

All classes of antimicrobial (macrolide, ketolide, and peptide deformylase inhibitor) bacterial protein synthesis inhibitors investigated in applicants' model have demonstrated the ability to reduce BAL cytokine/chemokines concentrations. The various antimicrobial agents studied, regardless of class, have all shown the ability to significantly reduce BAL concentrations of cytokine/chemokines compared with placebo (Hardy et al, 2003; Rios et al, 2004; Fonseca et al, 2005b; Rios et al, 2005). For the most part, all the agents have had a similar qualitative effect, while the quantitative effects have varied. This illustrates that the observed “immunomodulation” is not a “macrolide only” effect; this is important as mainly macrolide antimicrobials have been studied in this regard (Rubin et al, 2004). The noted cytokine/chemokine modulation seems to be present with bacterial protein synthesis inhibitors in general for therapy of M. pneumoniae infection. These agents may be dampening a virulence mechanism of m. pneumoniae, which then leads to an alteration in host response. Bacterial protein synthesis inhibitors may improve M. pneumoniae infection to a significant degree by the inhibition of mycoplasma CARDS TX protein production. While “direct” host-immunomodulation properties may exist for some of these agents, evidence has been provided that a “direct” host-immunomodulatory effect is unlikely with clarithromycin therapy in this specific model of disease (Hardy et al, 2003). Similarly, Kraft et al. (2002) have demonstrated that asthmatics infected with atypical bacteria benefit significantly more from clarithromycin therapy than asthmatics without evidence of atypical bacterial infection. The beneficial clinical effect of macrolide therapy on Pseudomonas aeruginosa respiratory infections is often postulated to be due to inhibition of protein synthesis with resultant suppression of pseudomonal virulence factors (Tateda et al, 1996; Tateda et al, 2000; Rubin and Henke, 2004; Wozniak and Keyser, 2004). Similarly, the relationship between these antimicrobials and M. pneumoniae may be analogous to that proposed between clindamycin and Streptococcus pyogenes virulence proteins (AAP, 1998).

The noted disassociation between the antimicrobial and anti-inflammatory response to bacterial protein synthesis inhibitors varies with agent. Azithromycin therapy had no significant effect on BAL concentrations of IL-6 and INF-γ in the applicants' M. pneumoniae model, while significant reductions in these cytokines were demonstrated with clarithromycin (a macrolide), cethromycin (a ketolide), and NVP-PDF713 (a peptide deformylase inhibitor) therapy (Hardy et al, 2003; Rios et al, 2004; Rios et al, 2005; Fonseca-Aten et al, 2005b). The mechanism for the difference in cytokine modulation by azithromycin compared with these other antimicrobials investigated in this model is unknown; however, azithromycin may interact with mycoplasma virulence properties (such as CARDS TX protein production) in a different manner than these other inhibitors of protein synthesis. Antimicrobial structural differences are known to produce differential inhibitory effects on protein synthesis and 50S ribosomal subunit assembly (Mabe et al, 2004). In addition, azithromycin therapy did not improve airway obstruction in the applicants' model, from an immunopathogenic perspective; this may have been a functional consequence of the lack of significant reduction in BAL concentrations of IL-6 and/or INF-γ with azithromycin therapy.

In the present invention, studies will be carried out to determine if the anti-inflammatory affect of bacterial protein synthesis inhibitors in M. pneumoniae respiratory infection varies with the inhibitors' ability (specific inhibitor, dosage of inhibitor) to decrease CARDS TX activity in the applicants' model of infection. This knowledge will facilitate the development of more effective therapies for reactive airway disease and asthma associated with M. pneumoniae. Additionally, this knowledge will assist in the development of therapies for other respiratory diseases for which macrolides and other bacterial protein synthesis inhibitors have been found useful independent of their traditional antimicrobial properties.

Project 2. Role of CARDS Toxin in M. pneumoniae Associated Asthma in Mice

Studies will be carried out to determine if the CARDS TX is responsible for acute, chronic and exacerbation of asthma. The BALB/c-ovalbumin model of allergic asthma will be used to test the following specific aims: 1) determine the contribution of the CARDS TX to the pathogenesis of M. pneumoniae associated allergic asthma using established murine models; 2) investigate the role of CARDS TX in the pathogenesis of asthma associated with M. pneumoniae infection. Mice will be infected with wild type M. pneumoniae or M. pneumoniae with a null mutation in the CARDS TX gene. Pathogenesis will be evaluated in the BALB/c mouse model with and without ovalbumin-induced airway hyper-responsiveness, to elucidate the role of CARDS TX in the context of the infectious model; and 3) investigate the activity of CARDS TX in vivo. Studies of CARDS TX-induced gene expression, localization/co-localization, and biochemical activity in vivo will also be carried out, using the BALB/c mouse model with and without ovalbumin-induced airway hyper-responsiveness.

### CARDS TX Elicits Cytopathology in Mammalian Cells.

PCR was used to amplify the cards tx open reading frame from M. pneumoniae clinical isolate S1. The amplified gene was cloned into a bacterial expression vector and recombinant protein expressed and purified.

CHO cells cultured in the presence of exogenous rCARDS TX displayed characteristic rounding and distinct vacuolization with disruption of monolayer integrity. Both low concentrations of rCARDS TX (10-50 ng/ml) and high concentrations of rCARDS TX (10-50 μg/ml) elicited overt cytopathological effect (CPE) but with different time requirements. Heat inactivation of rCARDS TX preparations (30 minutes at 60° C.) abolished CPE, which reinforced the cytotoxic properties of ‘heat labile’ CARDS toxin.

Seroconversion to CARDS TX in M. pneumoniae Infected Patients.

Human sera from individuals with M. pneumoniae-confirmed respiratory infections were screened for CARDS TX antibodies. ELISA results clearly confirm seroconversion to CARDS TX. It is apparent that high titers of anti-CARDS TX antibodies are generated in humans infected with M. pneumoniae, indicating that CARDS TX possesses important diagnostic and prognostic potential.

Anti-CARDS TX antibodies and PCR products are present in acute, convalescent, and chronic pediatric asthma patients. Using ELISA- and PCR-based methods for the detection of CARDS TX to examine clinical samples from pediatric asthma patients, forty three patients, ages 5-15 years who presented with acute asthma, were examined. Nasal washes (NW) were collected from 40 of the patients and serum from 42. Three to eight weeks later “convalescent” NW and serum samples were also obtained. Additionally, sera from 23 chronic asthmatics ages 6-40 years were included in the analysis. NW and sera were analyzed for antibodies to the CARDS TX or the P1 adhesin protein and by PCR for the CARDS TX or P1 gene. P1 is the major adhesin of M. pneumoniae and is considered the ‘gold standard’ for molecular diagnosis of M. pneumoniae. CARDS TX antibodies can be detected in the serum of 55% of acute asthmatics, 48% of the convalescent asthmatics, and 70% of the chronic asthmatics (Table 1). In contrast, P1 antibodies were detected by ELISA in only 26% of acute asthmatics, 4% of convalescent asthmatics and 26% of chronic asthmatics. Although there could be numerous explanations for the differences in the immune response to the CARDS TX or P1, what is clear is that correlation between M. pneumoniae infection and asthma is very impressive, and probably under appreciated due to the limitations of existing (i.e., non-CARDS TX based) diagnostics.

TABLE 1
CARDS TX antibodies and P1 antibodies in the serum of asthma patients

| | Acute asthma | Convalescent | Chronic |
|---|---|---|---|
| ELISA¹ | (42) | asthma (23)* | asthma (23) |
| CARDS | 23/42 (54.76%) | 11/23 (47.83%) | 16/23 (69.57%) |
| TX | | | |
| P1 | 11/42 (26.19%) | 1/23 (4.35%) | 6/23 (26.09%) |

¹Individual serum samples were analyzed by ELISA against equimolar concentrations of recombinant CARDS TX or recombinant P1 protein. Results represent the fraction of positives per total analyzed. Positive and negative results were determined based on the analysis of statistical significance of ELISA results from known positive and negative serum samples. Note that acute and convalescent categories represent the same group of patients, who presented with acute asthma and were re-studied 6-8 weeks later. The chronic patients represent a separate group who presented with clinically stable chronic asthma.

In contrast to antibody based assays, the presence of CARDS TX or P1 genes as determined by PCR of asthmatic patient samples more directly links M. pneumoniae to infectious and pathological processes. The CARDS TX gene was detectable at impressive levels in serum and nasal washes of acute and convalescent asthmatics and less so in chronic asthmatics. Altogether, these data suggest an involvement of the CARDS TX gene and M. pneumoniae with acute and convalescent asthmatics.

Inflammatory Responses in Mouse Lung to M. pneumoniae CARDS TX.

Concentrations of cytokines were evaluated in the bronchial alveolar lavage fluid (BALF) of BALB/c mice that were sedated and intranasally (IN) inoculated with 50 μg of rCARDS TX, or carrier solution as a negative control. After two days, BALF was collected and concentrations of cytokines and chemokines determined using the Luminex system or ELISA. We examined levels of IL-1β, IL-2, IL-4, IL-5, IL-6, IL-10, IL-12, IL-13, KC, TNF-α, IFN-γ, GM-CSF, IP10, TGF-β, MIP-1α, MIG and MCP-1. This immunologic profile represents inflammatory, TH1, TH2, growth factor, and chemotatic cytokines for which associations have been made with M. pneumoniae respiratory disease (7, 8, 18, 25, 38, 41, 45, 53). Statistically significant differences (p<0.05) in cytokine concentrations ranging from 4 fold to 800 fold were observed for IL-12 (6 fold), TNF-α_(600 fold, IL-6 (800 fold), GM-CSF (4 fold), IL-1β(5 fold), KC (4 fold), and Mip1a_(8 fold). No significant differences between carrier solution and CARDS TX in BALF were observed for IL-2, IL-4, IL-5, IL-10, IFN-γ, MCP-1, MIG, and IP-10. This cytokine profile, which is consistent with the profile observed in the mouse model of M. pneumoniae infection, is indicative of a Th1 type inflammatory response and clearly implicates CARDS TX as a mediator of proinflammatory responses in the mouse airway.

Based on what has been observed with M. pneumoniae infections, it was important to examine the histopathology that follows CARDS TX intoxication. Lungs of mice used for cytokine analysis were prepared for histopathology and scored using the system of Cimolai et al. (9). To determine the impact on lung pathology, lungs were removed and fixed in 10% neutral buffered formalin (NBF) prior to being embedded in paraffin, sectioned, and stained with H&E. No histologic abnormalities were present in control lungs, and heat inactivated toxin treated lungs showed minimal numbers of lymphocytes in peribronchiolar and perivascular sites).

Pathological findings in lungs of 2, 4, and 6 day CARDS TX-treated mice were diffuse and striking. In all TX-treated groups, peribronchiolar and perivascular infiltrates of mononuclear cells with a dominance of lymphocytes were identified. Lymphocytes varied in cell layer thickness in bronchiolar walls, but over 6 days, lymphocytes infiltrated adjoining alveolar walls, interlobular septae and subpleural sites. Using the system of Cimolai (9), the average score of intoxicated mice was 10, which is based on the degree of lymphocytic infiltration, including severe peribronchial, perivascular and parenchyma infiltration. These findings were reminiscent of pathology observed in CARDS TX intoxicated baboons and mice infected with M. pneumoniae as described herein. The carrier solution group (negative control) represented data from four animals (average score of 0) and the TX group was from five animals per time point.

Mycoplasma pneumoniae Produces CARDS TX in Infected Mouse Lungs.

To confirm that CARDS TX is produced in vivo, mice were infected IN with 10⁷ CFU of wild-type (WT) M. pneumoniae or cards tx null mutant. After 4 days and 18 days, lungs were harvested for immunohistochemistry and histopathology. Fresh frozen tissues (lungs) were prepared as described previously (16, 17) and 7 μM sections were cut and stained for the presence of M. pneumoniae and CARDS TX.

CARDS TX is produced in infected cells in vivo, and significant amounts of CARDS TX appear to accumulate in lungs during infection. Toxin can also be detected in areas of the lung where there is no apparent M. pneumoniae staining, suggesting that it is diffusible within infected tissues.

Intranasal Application of CARDS TX in Mice Leads to Pronounced Cellular Infiltration to BALF.

During a six-day experiment, mice treated with CARDS TX showed a robust infiltration of cells into BALF, whereas animals treated with heat-inactivated CARDS TX remained at baseline levels. Only slight differences were observed when either carrier solution or heat-inactivated TX was used as a control. Cellular levels of infiltration were determined by directly counting cells in BALF using a hemacytometer. FACS analysis of cells in BALF of intoxicated animals revealed a 3-5 fold increase in numbers of GR1+ granulocytes and a 3-10 fold increase in CD11b+, BM8+macrophages over the course of the experiment. Numbers of CD4+ and CD8+ T-cells, as well as CD19+ B-cells, did not change.

CARDS TX Induces Changes in Airway Resistance and Hyperresponsiveness to Methacholine Treatment

A common finding in patients with chronic M. pneumoniae infection or experimental animals with chronic M. pneumoniae infection is the development of obstructive airway disease. To test the impact of CARDS TX on pulmonary function, whole-body unrestrained plethysmography was used to monitor both airway resistance and airway hyperreactivity after methacholine treatment of intoxicated mice (18). BALB/c mice received IN application of 50 μg of CARDS TX or carrier solution, and two days later pulmonary function was assessed. Statistically significant (p<0.05) differences in both airway resistance and methacholine reactivity were observed, consistent with what is observed during M. pneunoniae infection. Altogether, these data suggest that CARDS TX may be responsible for many or all of the pathophysiological changes observed during both acute and chronic M. pneumoniae infections, including changes in cytokine expression, infiltration of the lung by immune cells, and airway dysfunction.

Project 3. Mycoplasma pneumoniae Infection in Patients with Chronic Asthma

The presence and titer of antibodies reactive against the CARDS toxin and the frequency of cards tx PCR positivity may be considered to be key indicators of disease status. This project will evaluate the prevalence of antibodies to CARDS toxin and detect cards tx DNA by PCR in nasal lavage, sputum and serum in various groups of patients with acute and chronic asthma. Chronic stable asthmatics, patients with acute exacerbation of asthma and a group of asthmatics with refractory asthma will be evaluated. The sensitivity of the CARDS toxin to the “gold” standard P1 assay for M. pneumoniae and the cellular and cytokine responses will be evaluated in these groups of asthmatic patients.

Inflammatory Responses in Baboon Lung to CARDS TX.

Two adult baboons (Papio) underwent bronchoscopy using an Olympus bronchoscope after sedation and oral intubation. Both animals were lavaged with 20 ml of saline with a return of 10 ml of BALF. Following acquisition of baseline BALF specimens from the left lower lobe, one animal received 50 μg of rCARDS TX in carrier fluid in the right lower lobe (RLL) bronchus, and the control baboon received the same amount of heat inactivated CARDS TX in the RLL. Thereafter, BALF specimens were obtained on days one and two from both lower lobes. All samples of BALF were analyzed for cytokine and chemokine expression utilizing a Luminex analysis of 21 cytokines/chemokines. Baboons were euthanized and necropsies performed. Sections of trachea, right and left lower lobe bronchi, and distal bronchi and bronchioles were obtained from both animals. Lung specimens were fixed in 10% formalin, paraffin processed for light microscopy, and stained with H&E. Clinically, the baboons remained afebrile with normal chest radiographs. Significant elevations in cytokine and chemokine concentrations were noted in the CARDS TX-treated animal at day 1 and 2, but not in the control: G-CSF (40 fold), IL1Ra (10 fold), IFN-α (75 fold), IL6 and IL8 (100 and 600 fold, respectively), MIP-1α (5 fold), and RANTES (9 fold). Histopathologically, trachea and bronchi of the heat-inactivated control showed focal epithelial loss and scattered eosinophils secondary to the lavage procedures. Distal bronchi and bronchioles showed intact epithelium and no mural inflammation, with only scattered small collections of bronchus-associated lymphoid tissue in some bronchiolar walls. In TX-treated baboon specimens, the trachea, right and left main bronchi and more distal bronchi showed extensive denudation of epithelium and increased numbers of lymphocytes and scattered eosinophils in the submucosa. A lymphocytic bronchiolitis was identified in numerous bronchioles, and foci of alveolar edema were present. These data indicate that the CARDS TX is capable of eliciting a response in the baboon that is consistent with what is observed in human M. pneumoniae infection and in mouse models of both infection and intoxication.

Seroconversion to CARDS TX in M. pneumoniae Infected Patients.

As mentioned above, M. pneumoniae infection induces an immune response similar to that observed with CARDS TX application to the lungs of the baboon. Since rCARDS TX also exhibited CPE and ADP-ribosylating activity in mammalian cells, M. pneunoniae-infected humans were screened for CARDS TX antibodies. This would provide direct evidence for both the synthesis of CARDS TX in vivo and its immunogenic potential. Acute and convalescent-phase sera obtained during an outbreak in 1993 (Wilford Hall USAF Hospital, San Antonio) from 9 patients with serodiagnosed and confirmed airway disease caused by M. pneumoniae were examined. Sequential serum samples, which originally showed seroconversion to total M. pneumoniae protein antigens by immunoblot, were tested. For performing ELISAs human sera were diluted in 1% BSA in PBS and established ELISA methodologies were used. Briefly, Dynex HBX4 96-well microtiter plates were coated with rCARDS TX in carbonate-bicarbonate buffer (pH 9.6). Serum samples were assayed in duplicate or triplicate at 1/100 to 1/1600 dilutions. Secondary antibody was diluted 1/1000-1/2000 in 1% BSA/PBS. Plates were incubated for one and a half hours at room temperature and washed, and pNPP [alkaline phosphatase substrate (Sigma Fast)] was added to each well. Plates were developed for thirty minutes at room temperature and then spectrophotometric readings were taken at 405 nm using a microplate reader (Dynatech HBX). The mean optical density of wells containing antigen incubated with conjugated antibody, but not human sera samples, was subtracted from optical densities of human sera. ELISA results clearly confirm seroconversion to CARDS TX. It is apparent that high titers of anti-CARDS TX antibodies are generated in humans infected with M. pneumoniae, which has direct diagnostic applications. All sera were tested against purified pertussis toxin (PTX) S1 subunit (List Biological Laboratory, Inc., Campbell, Calif.) as antigen and shown to exhibit no (minimal) reactivity.

CARDS TX-Based PCR Detection of M. pneumoniae in Simulated Clinical Sputum Samples and Chronic Asthma Patient-Derived Nasopharyngeal Washes (NWs).

M. pneumoniae strains and other Mycoplasma species were grown in SP4 broth, harvested at mid-to-late exponential growth phase and stored at −80° C. Sputum samples were collected from healthy individuals, and known amounts (in triplicate) of SP4-broth grown M. pneumoniae cells were added. These ‘spiked’ test preparations were subjected to DNA extraction utilizing a commercial kit (QiaAmp DNA Blood Mini Kit, Qiagen). Based on the cards tx sequence, unique regions were selected for generation of oligonucleotide primers. Distinct bands of expected size were observed with a detection limit of 10×10¹⁵ g, which is equivalent to ≈11 genome copies (816 kbp). Existing P1 and ribosomal RNA PCR primer-based assays are less sensitive (33). The PCR amplified cards tx gene product (368 bp) was then hybridized with internal probes to establish specificity. To further evaluate cards tx PCR specificity, four distinct clinical strains of M. pneumoniae, isolated between 1993 and 2001, gave rise to identical band products. When amplification of 10 pg of DNA from Mycoplasma genitalium (G37) and Mycoplasma penetrans (GTU54) were performed, no bands related to M. pneumoniae cards gene were observed. PCR was also employed to amplify cards tx sequences from NWs of pediatric patients with chronic wheezing, and in 3 of 8 specimens examined, unequivocal evidence was obtained for the presence of cards tc DNA. The current PCR assay for M. pneumoniae, which amplifies unique P1 adhesin gene sequences, was weakly positive in only one patient. These studies indicate that the cards tx gene sequences, under the conditions of the assays described herein, are superior target molecules than P1-targeted primer sets (or ribosomal RNA targets) in PCR assays for detecting and linking M. pneumoniae to both acute and chronic airway diseases, including asthma.

Anti-CARDS TX Antibodies and PCR Products are Present in Acute, Convalescent, and Chronic Pediatric Asthma Patients.

In contrast to antibody based assays, detection of unique CARDS TX or P1 gene sequences as determined by PCR indicates the presence of M. pneumoniae (or at least M. pneumoniae DNA) in specific tissues or body fluids. A CARDS TX PCR assay was used to detect the presence of CARDS TX gene sequences in this patient population, which was compared to the existing ‘gold standard’ published P1 PCR assay. The CARDS TX gene was amplified in serum of 17% of acute asthmatics, 13% of convalescent asthmatics and 4% of chronic asthmatics. P1 was present in serum of 5% of acute asthmatics and in 0% of convalescent or chronic asthmatics (Table 2). When NW material was examined by PCR, CARDS TX gene was present in 30% of acute asthmatics, and 13% of convalescent asthmatics. P1 was detectable in 5% of acute asthmatics and undetectable in convalescent asthmatics. NW samples from chronic asthmatics were unavailable. These data demonstrate that CARDS TX gene sequences can be detected in respiratory secretions of patients, for correlation with symptomatology and disease pathogenesis.

TABLE 2
Detection of CARDS TX and P1 by PCR in the serum and nasal washes of
asthmatics

| | Acute | | Chronic |
|---|---|---|---|
| PCR | asthmatics | Convalescent | Asthmatics |
| serum | (n = 42) | asthmatics (n = 23) | (n = 23) |
| CARDS | 7/42 (16.67%) | 3/23 (13.04%) | 1/23 (4.3%) |
| TX | | | |
| P1 | 2/42 (4.8%) | 0/23 (0%) | 0/23 (0%) |
| | Acute | | Chronic |
| PCR | asthmatics | Convalescent | Asthmatics |
| NW | (n = 40) | asthmatics (n = 23) | (n = 0) |
| CARDS | 13/43 (30.23%) | 3/23 (13.04%) | N/A |
| TX | | | |
| P1 | 2/43 (4.65%) | 0/23 (0%) | N/A |

Serum and nasal wash samples from each category were subjected to PCR for cards tx or p1 genes as described herein for studies with simulated clinical sputum samples and chronic asthma patient-derived nasopharyngeal washes (NWs). Acute and convalescent categories represent the same group of patients, who presented with acute asthma and were re-studied 6-8 weeks later. The chronic patients represent a separate group who presented with clinically stable chronic asthma.
N/A, not available.

Altogether these data suggest that the CARDS TX is expressed during human infection, and the presence of anti-CARDS TX antibodies and cards tx sequences can be detected in an unusually high number of asthmatics.

M. pneumoniae Produces CARDS TX in Lungs.

These clinical data suggest that detection of CARDS TX in asthmatics (based upon serological and PCR evidence) is significant and may be indicative of a greater correlation of M. pneumoniae infection in this patient population. However, these data do not prove that CARDS TX is expressed in the lungs of asthmatics. To confirm that CARDS TX is produced in vivo, mice were infected intranasally (IN) with 10⁷ CFU of the S1 clinical strain of M. pneumoniae or cards tx null mutant. After 4 days and 18 days, lungs were harvested for immunohistochemistry (using anti-CARDS TX polyclonal antibodies raised against recombinant protein) and histopathology. Fresh frozen lung tissues were prepared and stained for the presence of M. pneumoniae and CARDS TX. These studies clearly indicated that, in infected mice, there is significant CARDS TX expression in lungs that co-localizes with M. pneumoniae. Significantly, co-localization was not observed with cards tx null mutant. These studies also indicate that CARDS TX may be a diffusible virulence factor in vivo.

The identification of CARDS TX and card tx gene and their potentially superior role as a diagnostic target compared to existing reagents for M. pneumoniae should help improve the clinical management M. pneumoniae-associated asthma. More significantly, CARDS TX may be the etiologic factor in M. pneumoniae-associated asthma and other related airway diseases. The cards tx gene and anti-CARDS TX antibodies are present in serum and respiratory secretions of asthmatics, as well as people with M. pneumoniae associated pneumonia. Using a primate animal model, applicants have shown that CARDS TX elicits cytokine and chemokine profiles and histopathological changes observed with M. pneumoniae infection. Similar results were observed in murine models. Finally, these studies demonstrate that, while very little CARDS TX is produced during in vitro broth culture, mice infected with M. pneumoniae produce detectable amounts of CARDS TX, and rCARDS TX ADP-ribosylates and induces CPE in mammalian cells and pathology in animal models.

Project 4. Biochemical, Molecular and Immunological Characterization of the M. pneumonia CARDS Toxin

These studies will be carried out to characterize CARDS TX-mediated ADP-ribosyl transferase (ART) activity through detecting CARDS TX minimal domain(s) and amino acids essential for enzymatic activity; and identifying the mammalian proteins that are ADP-ribosylated by CARDS TX. Transcriptional and proteomic analysis will be carried out of CARDS TX in wild type strains and in M. pneumoniae strains having their CARDS TX promoter fused to GFP and luciferase under different environmental conditions. CARDS TX epitopes that serve as antigenic and diagnostic determinants in humans and mice will be mapped and those epitopes capable of inducing neutralizing antibodies will be identified. The goal is to develop effective strategies to diagnose, treat and prevent asthma and related airway diseases in a substantial population of both children and adults.

Characterization of CARDS TX as a Bona Fide Virulence Determinant.

The amino-terminal region of the CARDS TX exhibits amino acid identity (27% of amino acids 1 to 226) with Bordetella pertussis toxin S1 subunit. However, the remaining amino acid residues of MPN372 (amino acids 227 to 591) shared no homologies with other subunits of pertussis toxin, any known toxins, or reported protein sequences.

Cloning and Site-Directed Mutagenesis of the CARDS TX Gene.

Since the quantity of CARDS TX protein is very limited in SP4 grown cultures of M. pneumoniae, PCR was used to amplify the mpn372 coding region from M. pneumoniae clinical isolate S1 as described herein. The sequence of mpn372 has eight UGA codons at amino acid positions 148, 195, 233, 364, 392, 450, 462 and 508. Therefore, single strand overlap extension polymerase chain reaction (SOE-PCR) was used for site-directed mutagenesis to change each UGA codon into UGG in order to express full-length rCARDS TX protein in E. coli.

Expression and Purification of CARDS TX.

Utilizing NdeI and BamHI restriction sites incorporated in oligonucleotide primers, the entire fragment containing cards tx (i.e., mpn372) coding region was cloned into E. coli His₁₀-tagged expression vector, pET19b. Since the NdeI site (CATATG) in the vector overlaps an ATG start codon, cloning mpn372 into the NdeI site places mpn372 in perfect register with the vector-derived ribosome binding site. Therefore, recombinant proteins are expressed full-length with an amino terminal His₁₀-tag. After cloning the mpn372 PCR fragment into pET19b and confirming the identity of the cloned fragment by DNA sequencing, recombinant plasmid designated pET-MPN372 was used to transform E. coli strain BL21 (λDE3). Transformants (5000 ml culture) were grown to mid-log phase before inducing CARDS TX expression by addition of IPTG to a final concentration of 1 mM. After four hours, cells were harvested by centrifugation at 8000 g for 15 min at 4° C., and the pellet was resuspended in 50 mM phosphate buffer pH 8.0, containing 300 mM NaCl, 10 mM imidazole and complete, EDTA-free protease inhibitor (Sigma). Cells were disrupted by sonication; cellular debris and membranes were pelleted and discarded; supernatant was mixed with Ni-NTA agarose slurry and left on a rocker at room temperature for one hour; and then the slurry was loaded onto a Ni-NTA agarose (Qiagen) packed column, which was extensively washed with imidazole. Finally, rCARDS TX was purified in a single step elution with 250 mM imidazole, desalted and concentrated. Protein concentrations were estimated using a BCA protein assay kit (Pierce), aliquoted and stored at −80° C.

Expression of Recombinant Proteins in Lipid-A Deficient E. coli Strain BL21 (λDE3).

Wide-scale screening of E. coli-expressed recombinant bacterial proteins is complicated by the inherent contamination of recombinant proteins with endotoxin. This is especially serious when recombinant proteins are being evaluated in endotoxin-sensitive mammalian cell lines or animal models. Studies examining the role of LPS in the induction of inflammation have demonstrated that the lipid A portion of LPS is responsible for the majority of the pro-inflammatory properties of LPS⁵². Inactivation of LpxM in E. coli⁵² and Salmonella⁵³ results in strains which grow normally but produce a non-myristylated LPS (nmLPS); the latter exhibits markedly diminished ability to induce cytokine production in vitro and in vivo³. In order to express recombinant proteins without lipid A contamination, a lpxM mutant E. coli strain BL21 (λDE3) was constructed⁵⁴. All mycoplasma recombinant proteins in applicants' laboratory, such as CARDS TX and elongation factor-Tu (EF-Tu), are currently expressed and purified from lpxM-inactivated E. coli BL21 (DE3)⁵⁴(provided by Dr. Jean-François Gauchat). Further, all purified recombinant proteins are routinely passed through sequential polymixin columns to reduce remaining ‘endotoxin’ contamination before use on cell lines and in animal experiments described herein. Also, Limulus assays are performed to determine endotoxin concentrations in each recombinant preparation. Endotoxin is measured with the Limulus amebocyte lysate system (Associates of Cape Cod, East Falmouth, Mass.) according to the manufacturer's directions. All preparations of rCARDS TX and other recombinant mycoplasma proteins/peptides are analyzed for endotoxin levels. These recombinant preparations, which are measured at concentrations of 50 μg recombinant protein/50 μl contain endotoxin levels at or below the minimal detection levels of the assay (0.1 EU/ml). The assay is linear over a range of 0.1-1.0 EU/ml.

Generation of Mouse Anti-CARDS TX Polyclonal Antibodies Against Full Length rCARDS TX.

Six week old Balb/c female mice (n=10) were bled and screened by immunoblot using rCARDS TX to determine pre-existing antibodies. No immune reactivity was observed, and mice were immunized with 47.5 μg of rCARDS TX mixed with complete Freund's adjuvant intraperitoneally (IP). Three weeks post-immunization, animals were boosted IP with 50 μg rCARDS TX mixed with incomplete Freund's adjuvant. Three weeks later, animals were screened by immunoblot using rCARDS TX or whole M. pneumoniae cell lysates. In the latter case, highly concentrated mycoplasma preparations were required to detect CARDS TX because of the very low quantities of CARDS TX protein synthesized by M. pneunoniae during growth in SP4 broth culture. All immunized mice exhibited strong serological reactivity to rCARDS TX, and to CARDS TX from whole mycoplasma concentrated lysates. Mice were again immunized IP with 50 μg rCARDS TX mixed with Freund's incomplete adjuvant. Sera were collected 2 weeks later, re-screened by immunoblot and ELISA, and pooled. Hybridomas can be generated using rCARDS TX-immunized mice.

Identification of CARDS TX Shared Motifs and Topology Relative to ADP-Ribosylating Toxins.

Primary amino acid sequence alignment shows homologies between PTX S1 subunit (>27% identity over >239 residues) and CARDS TX. Although bacterial ADP-ribosylating enzymes do not share extended amino acid conservation, they exhibit a conserved active site scaffold (Table 3). For example, alignment of the primary amino acid sequence of CARDS TX with PTX S1 and others indicates that CARDS TX contains three conserved motifs common to bacterial ARTs, which are necessary for catalysis⁵⁵⁻⁵⁷. These three motifs are: i) a potential catalytic glutamate observed at position 132. (ii) a β/α region with a serine-threonine-serine (STS) motif (residing at positions 49 and 51) needed for structural integrity of the NAD binding site and (iii) a conserved arginine residue at position 10 necessary for NAD binding in many ADP-ribosyltransferases (ARTs). Further, another amino acid which is conserved in CARDS TX is histidine in position 34, which corresponds to His 35 in PTX and His 44 in LTX and CTX. Conservation of these motifs in CARDS TX, plus other similarities with PTX S1, reinforce the link between CARDS TX and ART activity. Mono-ADP-ribosylation is a post-translational modification of proteins, shared by eukcaryotes and prokaryotes, which modulates protein function. Mono-ADP-ribosyltransferases catalyze the transfer of ADP-ribose group of β-nicotinamide adenine dinucleotide (NAD⁺) to a specific amino acid in target protein acceptors with the simultaneous release of nicotinamide⁵⁸.

rCARDS TX Elicits Cytopathology (CPE) in Chinese Hamster Ovary (CHO) Cells.

To monitor CPE associated with CARDS TX, CHO cells were used as the indicator system since PTX is known to elicit a rounded and clustered appearance in these cells⁵⁹. Endotoxin-deficient E. coli⁵⁴ were used to generate high yields of rCARDS TX with little to no detectable endotoxin. CHO-K1 cell (ATCC) monolayers were grown to 60-75% confluence in 25-ml flasks using F12-K medium supplemented with 5% fetal bovine serum. This medium was replaced by fresh F12-K medium w/o serum but containing filter-sterilized (0.22 μm) endotoxin free rCARDS TX, or heat-inactivated rCARDS TX (30 minutes at 100° C.) or rEF-Tu_{Mp} as negative controls for 2 to 4 hours in a CO₂ incubator at 37° C. Then, serum was added to each culture and incubation continued for 16-48 hours. CHO cells were observed every 6-12 hours for morphological changes. CHO cells cultured in the presence of exogenous rCARDS TX displayed characteristic rounding and distinct vacuolization with disruption of monolayer integrity.

Cytopathology was slow to develop at low concentrations of rCARDS TX (10-50 ng/ml), requiring approximately 16-28 hours, while higher concentrations of rCARDS TX (10-50 μg/ml) elicited overt CPE in 4-12 hours. Heat inactivation of rCARDS TX preparations abolished CPE, which reinforced the cytotoxic properties of ‘heat labile’ CARDS TX (i.e., endotoxin is heat stable under these conditions). Also, rEF-Tu preparations (additional negative control) were without cytopathologic effect.

rCARDS TX Possesses ADP-Ribosyltransferase Activity.

ART activity of rCARDS TX was assayed by determining incorporation of ³²P ADP-ribose moiety (from a [³²P] NAD) into indicator cell proteins as described for PTX⁶⁰. Briefly, CHO-K1 cell extracts were incubated with (5 to 40 μg) or without rCARDS TX for 10-30 min in 100 μl of reaction mixture containing 0.1M Tris-HCl (pH 7.5), 0.1mM ATP, 0.1 mM GTP, 10 mM thymidine, 20 mM dithiothreitol (DTT) and 10 μM α³²P-NAD (10 Ci/mmol). Radiolabelled proteins were TCA precipitated and dissolved in SDS-PAGE sample buffer and heated for 3 min at 100° C. Aliquots were subjected to SDS-PAGE through 4% to 12% NuPAGE gels (Invitrogen) using MES or MOPS as running buffers and standard protocols. After electrophoresis, gels were transferred to nitrocellulose membranes, dried and exposed to Kodak X-OMAT film for 24 h. CHO cell extracts from non-CARDS TX treated cells possessed very weakly radiolabelled bands with apparent molecular masses ranging from 20 to >90 kDa; i.e., mono-ADP ribosylation occurs in CHO cells via ‘inherent’ mono ARTs. CHO cell extracts treated with CARDS TX possessed intensely radiolabeled, ADP-ribosylated proteins with apparent molecular masses at 45, 28, 26 and 21 kDa. Studies were undertaken to determine whether sulphydryl agents might influence CARDS TX activity since many bacterial ADP-ribosylating toxins undergo enzymatic activation following reduction of a disulfide bridge⁶¹ and the primary structure of CARDS TX contains six cysteine residues. Indeed, the formation of ADP-ribosylation was markedly increased by the presence of DTT, which suggests that CARDS TX ADP-ribosyltransferase activity is DTT-dependent, similar to cholera⁶² and pertussis toxins⁶³. The absence of GTP and ATP revealed less noticeable effects on ADP-ribosylation of target proteins. In parallel studies, intact CHO and HEp-2 cells were grown in 25 ml tissue culture flasks, incubated with or without CARDS TX (5 to 40 μg) at 37° C. for 16 h, and cell extracts were prepared. To these cell extracts, additional rCARDS TX was added along with reaction mixture containing ³²PNAD. Cell extracts of non-intoxicated and intoxicated intact human HEp-2 cells were also examined for ADP-ribosylation. Intact ‘intoxicated’ and untreated control HEp-2 cells were recovered from plates, washed in PBS, and suspended in lysis buffer [25 mM Tris-HCl (pH 7.5)]. Cell-free lysates were prepared and supernatants were used as the source of CARDS TX-sensitive ADP-ribosylated target proteins using the in vitro ADP-ribosylation assay. Cell extracts prepared from non-CARDS TX treated intact cells contained three prominent radiolabeled proteins with apparent molecular masses of 45, 42 and 26 kDa that were specifically ADP-ribosylated by rCARDS TX. Analysis of host proteins from the intoxicated, intact cell preparation (i.e., HEp-2 cells pre-incubated with 5 to 40 μg rCARDS TX for 16 h) showed a marked decrease in radioactivity incorporated into 45, 42 and 26 kDa target proteins (because these proteins had already been modified by CARDS TX previously added to intact HEp-2 cells and were now inaccessible to further ADP-ribosylation). There was also decreased intensity of radiolabeling observed in several other high and low molecular weight bands. When rCARDS TX was heat-inactivated or not added exogenously to intact HEp-2 cells, target proteins in HEp-2 cell extracts were available for ADP-ribosylation during the in vitro reaction with rCARDS TX. These studies demonstrate that full length rCARDS TX elicits CPE (cell rounding, vacuolization and disruption of cell monolayers) and ADP ribosylates specific target proteins in CHO and HEp-2 cells.

Polymorphism in Coding Region of CARDS TX.

CARDS TX gene mpn372 (only one copy per genome) was amplified from four clinical M. pneumoniae isolates (designated S1, L2, J1 and RJL1) plus reference laboratory strain (B9/M129) by means of primers, which included 10 bp upstream of the start codon and 10 bp downstream of the stop codon. 10 ng of chromosomal DNA from each strain underwent 12 PCR cycles in the presence of high fidelity Taq DNA polymerase as follows: 94° C. for 30 s (denaturation), 56° C. for 1 min (annealing), 68° C. for 2 min (extension). Polymerase chain reaction products were analyzed on 0.8% agarose gel and compared with 1 kb-plus DNA ladder molecular weight markers (Invitrogen). Amplified fragments were eluted using a Qiagen elution kit, cloned into a TOPO TA cloning vector and transformed into TOP 10 cells. Plasmids with inserts were sequenced by automated DNA sequencing performed on an Applied Biosystems DNA Sequencer model 373A by means of plasmid specific primers (M13 forward and reverse). Sequences were analyzed and translated to amino acids and compared with reference strain M129 using the ‘blast two sequences program’. Nucleotide and translated amino acid sequences of CARDS TX from reference strain B9/M129 showed 100% perfect match to published sequences of M. pneumoniae (M129) genome. However, the translation of nucleotide sequences of clinical isolates revealed changes in amino acids at positions 38, 245, 308, 371, 391 and 392. All clinical isolates exhibited changes at amino acid position 371 (Ile→Ser). JL had only one change at amino acid position 371 (Ile→Ser). RJL1 had one additional change (compared to JL) at aa position 392 (Trp→Arg). L2 had one additional change (compared to JL) at aa position 245 (Asp→Gly). S1 had three additional changes (compared to JL) at aa positions 38 (Leu→Pro), 308 (Ser→Pro) and 391 (Phe→Ser). These differences are interesting and can serve to link sequence polymorphisms with epidemiological and pathogenic observations in the future.

CARDS TX Gene Locus and Promoter Mapping.

Up- and downstream, the CARDS TX locus is flanked by genes encoding hypothetical proteins of unknown function. In contrast to mpn372, adjacent proteins are encoded by the complementary strand, and the presence of all corresponding transcripts has been demonstrated by RT-PCR. This organization of genes suggested a monocistronic transcript for mpn372, and promoter mapping was performed. A transcriptional start of mpn372 message was detected by primer extension (PE). Based on M. pneumoniae genomic DNA sequences⁸, forward primer 5′-ACCACAGCAGCGAACCGAGAATGG-3′ (SEQ ID NO:113) and reverse primer, MPN372PE, 5′-GCTGTGGGTGTTTCTGAAGTGGAA-3′ (SEQ ID NO:114) were designed. The corresponding upstream region was PCR amplified and cloned, and purified plasmid DNA was used to generate the sequence ladder. The same ³²P-labeled reverse primer (MPN372PE) was used in PE to identify the transcriptional start (5′-TTTTGAAGTTTTTAATTTGTAAAATTTCATTTTTTAAAAATG-3′) (SEQ ID NO:115). All products were electrophoresed on 6% sequencing gels. A short 5′ untranslated region (portion of transcript between transcriptional start and ATG-codon) was located 5 nucleotides downstream of the presumed-10 element. The identical single transcriptional start was detected when RNA was isolated at different mycoplasma growth phases and used in PE.

CARDS TX Location in Mycoplasma Membrane.

To assess the cellular location of CARDS TX in M. pneumoniae, immunoblot analyses were performed on cell extracts of clinical isolate S1 and reference strain M129. Whole cell lysates, cytoplasmic, membrane and culture supernatant fractions obtained from each strain during mid-to-late exponential growth phase were probed using antiserum raised against rCARDS TX. Immunoreactive 68-kDa bands were detected in total extracts and cytoplasmic and membrane fractions, but not culture supernatants.

To briefly summarize, M. pneumoniae CARDS TX was expressed in E. coli and rCARDS TX was purified under endotoxin-free conditions. Endotoxin-free rCARDS TX was shown to elicit cytopathology in CHO cells and possesses ADP ribosyltransferase (ART) activity. The CARDS TX gene, mpn372 or cards tx, is not organized in an operon and has its own transcriptional start at 10 bp upstream of the start ATG codon. The mpn372 gene was sequenced in different clinical isolates, which indicated the occurrence of limited polymorphisms. Also, CARDS TX is localized in the cytosolic and membrane fractions and some of the CARDS TX is surface exposed but no toxin is detected in the supernatant of in vitro grown mycoplasmas in SP4 broth. Contact of M. pneumoniae with mammalian cells or the in vivo microenvironment provides a considerably different set of signals that up-regulate CARDS TX transcription and translation.

Differential Gene and Protein Expression in M. pneumoniae with Special Focus on CARDS TX.

Pulse-chase analysis was used to identify up- and down-regulated translated products of M. pneumoniae during heat shock conditions. Macroarrays were also used to examine mycoplasma genes that were differentially regulated during various stages of growth. In order to isolate CARDS TX mutants, mini transposon 4001 mutagenesis was employed.

Characterization of M. pneumoniae Protein Expression at Different Temperatures.

The general properties of heat shock response were examined in M. pneumoniae by using pulse-labeling of mycoplasma proteins and one-dimensional SDS-PAGE. When growing cultures of M. pneumoniae were shocked by transfer from 37° to 42° C., there was a marked suppression of synthesis of specific proteins and a striking enhancement of synthesis of a small number of heat shock proteins with mobilities ranging from 58 to 98 kDa. Although no M. pneumoniae heat shock protein has yet been extensively characterized, two of these proteins appear immunologically related to the heat shock proteins, Dnak and ClpB. Thus, two categories of heat shock response proteins could be distinguished in these experiments; synthesis of one group of proteins declined markedly, and a second group of proteins continued to be synthesized and at elevated levels for about 6 h after heat shock.

Characterization of M. pneumoniae Gene Expression at Different Growth Intervals.

Differential expression at mRNA levels of M. pneumoniae genes of special relevance to this invention were measured: five genes (mpn372 [cards tx], tuf; gap, pdhA and pdhB) encoding proteins involved in M. pneumoniae binding to host targets, i.e., SP-A or fibronectin, and two housekeeping genes (enolase [eno] and NADH oxidase [nox] which serve as normalizers. Transcriptional levels of these genes were compared by DNA array at different mycoplasma growth phases. Gene-specific primers were designed based on the published M. pneumoniae genome. Gene fragments (404-607 bp depending upon gene size) were amplified using Platinum Taq DNA polymerase as suggested by the manufacturer (Invitrogen), and 250 ng of each product were blotted onto Zeta-Probe membranes (Bio-Rad Laboratories). Total RNA was isolated from M. pneumoniae cultures grown at 37° C. at early, mid-log, and late-log phases using Tri reagent (Sigma). Purified RNAs were quantified and treated with DNaseI (Gibco BRL), and cDNAs were synthesized using gene-specific reverse primers and simultaneous incorporation of [³²P]dCTP. After termination of reverse transcription, samples were treated with RNase H, and unincorporated nucleotides were removed by gel filtration using G-25 Sephadex column chromatography (Roch). Eluted cDNAs were used as probes in hybridization reactions. Hybridization was performed in solution with 50% formamide for 18 hours at 42° C. Each probe was hybridized to three identical membranes in corresponding volumes of hybridization solution (9 ml/membrane). After post-hybridization washes, all membranes were sealed in plastic bags, and images obtained. Different patterns were observed with increasingly strong levels of transcriptional activities from early to late log phase in all test genes, except for mpn372. In the latter case, levels of mpn372 transcript were highest (although very low levels of transcript were detected) during early to mid-log phases of culture growth, which decreased dramatically during late-log phase. The surprising demise of mpn372 expression during late log growth in SP-4 medium is consistent with the very low levels of CARDS TX detected in broth cultures.

Inactivation of CARDS TX Gene mpn372 using Mini-Tn4001.

Although transposons are used to generate insertional mutations for mapping and/or functional studies, transposons or their insertional elements may continue to transpose around the genome, resulting in multiple insertions and preventing clear interpretation of experimental data⁷⁰. This is particularly relevant when transposons are used in heterologous genetic systems (such as mycoplasmas) where control of the transposase may be compromised. To circumvent this problem, minitransposons have been constructed with an antibiotic resistance marker flanked by inner and outer inverted repeat transposon sequences. In this case, the transposase gene is located outside the inverted repeats to trap the transposon at the initial site of integrations. To avoid multiple insertions, transposon, pMT85-miniTn4001, was used (provided by Dr. Herrmann). pMT85-miniTn4001 has a colE1 plasmid origin of replication and a selectable gentamicin/kanamycin marker within Tn4001 to allow for direct rescue of chromosomal sequences adjacent to the insertion site. Specific properties of pMT85-miniTn400 were described in a poster presentation by the Herrmann lab (International Organization for Mycoplasmology Meeting, July 2005, Athens, Ga.). pMT85-miniTn400 was transformed into competent mycoplasmas by electroporation as described⁶⁹. After growth in selective medium (2 passages), the complete mixture of amplified transposon mutants was collected and M. pneumoniae cell suspensions were passed through 25-gauge needles several times and filtered through membrane filter units with 0.45-μm-pore size (Millipore) to remove mycoplasma aggregates. Then, mycoplasma cells were diluted in SP4 medium and plated on SP4 agar containing 80 μg/ml gentamicin. Chromosomal DNAs from individual Tn-integrated colonies were HindIII digested, self-ligated and transformed in E. coli Top10. Plasmid DNAs were isolated and subjected to DNA sequencing in order to identify the place of integration of the transposon in the chromosomal DNA of M. pneumoniae. Recently, among hundreds of colonies screened, a M. pneunoniae transposon mutant was isolated that had integration in the cards tx gene. By using primers specific for cards tx, mini-Tn-4001 integration was confirmed, and SDS-PAGE/immunoblot analysis confirmed that CARDS TX synthesis was absent in the mutant strain.

To briefly summarize, these studies demonstrate the existence of transcriptional and translational regulation in M. pneumoniae under different experimental conditions, which in turn set in motion an investigation of differential regulation of CARDS TX. CARDS TX is believed to be up-regulated when M. pneumoniae contacts host cells or in vivo microenvironments; based upon observations from the studies described herein. By using mini-Tn-4001 a CARDS TX null mutant of M. pneumoniae was isolated.

Collection of M. pneumoniae Infected Patient-Sera.

Acute and convalescent-phase sera obtained during an outbreak in 1993 (Wilford Hall USAF Hospital, San Antonio) from 9 patients with serodiagnosed and confirmed airway disease caused by M. pneumoniae were examined. Sequential serum samples, which originally showed strong seroconversion to M. pneumoniae antigens by immunoblot, were tested.

Immune Response of M. pneumoniae Infected Patients to CARDS TX.

For performing ELISAs test sera were diluted in 1% BSA in PBS. Individual wells of Immulon 4 HBX Immunoplates (Dynox) were coated at 4° C. overnight with 50 μl of rCARDS TX (or rPDH-B_{Mp} or rEF-Tu_{Mp} for comparative purposes), and diluted (50 ng to 3 μg/well) in carbonate-bicarbonate buffer (32 mM Na₂CO₃, 64 mM NaHCO₃). Plates were washed, 100 μl of 1% (wt/vol) BSA in PBS added to each well, and plates further incubated for 2 h at room temperature. After washing, 50 μl of diluted human sera were added to each well. Then, plates were incubated for 2 h at room temperature, washed, and 50 μl of goat antibodies reactive against human chain conjugated to AP (Zymed; 1:1000 dilution) were added to each well. Plates were incubated for 1.5 h at room temperature, washed, 50 μl of substrate solution [p-nitrophenyl phosphate (PNPP)/0.1M Tris pH 9.6] added, and plates incubated at room temperature for 30-60 min prior to A₄₀₅ determinations. M. pneumoniae-positive sequential sera and negative serum controls were assayed in duplicate or triplicate at 1/100 to 1/1600 dilutions to obtain a standard curve. The mean optical density of wells containing antigen incubated with conjugated antibody, but not human serum samples, was subtracted from optical densities of human sera values. ELISA results clearly confirm seroconversion to CARDS TX. Immunoblots were also performed on test sera, which reinforced these observations. Acute-phase sera exhibited mild reactivity to rCARDS TX, while sequential ‘convalescent’ sera obtained at 14 and 28 days after the initial serum draw demonstrated marked seroconversion to CARDS TX. Pooled sera from 20 healthy individuals possessed very low reactivity to CARDS TX.

Immune Response of Patients During Acute and Convalescent Stages of Asthma to CARDS TX and Adhesin P1 of M. pneumoniae.

To establish a link between M. pneumoniae, CARDS TX and asthma pathogenesis, acute and convalescent phase sera from individuals diagnosed with asthma were analyzed, which were part of a long-term study in Dallas. Recombinant CARDS TX and P1 immunodominant carboxy domain were used in an ELISA format as described herein. For negative controls, pooled human sera were used. In the acute phase, sera from 42 patients were tested. 23 out of 42 sera (54%) were positive by ELISA for CARDS TX, and 9 out of 42 patients (21%) were positive for P1. Among the 9 P1-positive patients, 7 were positive for CARDS TX. Thus, by pooling the CARDS TX and P1 positive patients, 25 (59%) were shown to be positive for M. pneunoniae infection during the acute phase. In the convalescent phase, era from 23 patients (only 23 of the 42 patients had ‘paired’ sera; i.e., both acute and convalescent sera) were tested, and 10 out of 23 patients (43%) were positive for CARDS TX while only 1 out of 23 (4.3%) was positive for P1. Among the 10 CARDS TX positive convalescent patients, 8 were previously positive during the acute phase (i.e., among the CARDS TX positive convalescent patients, 80% were previously positive during the acute phase).

The extent of CARDS TX positivity among patients categorized as asthmatics is truly intriguing, and the improved diagnostic capability that CARDS TX appears to provide was unexpected. Further studies and correlations are expected to provide additional insights concerning asthma diagnosis, patho-physiology and susceptibility, prognosis, treatment, etc. Studies are underway to develop a next generation diagnostic for M. pneumoniae based upon CARDS TX as target.

In a separate preliminary study, PCR of unique cards tc sequences in respiratory tract samples, including nasal washes and bronchoalveolar lavage fluid, indicated that 45% of adults with asthma were positive (only 2% of the control group were PCR-positive for cards tx).

Although the present process has been described with reference to specific details of certain embodiments thereof, it is not intended that such details should be regarded as limitations upon the scope of the invention except as and to the extent that they are included in the accompanying claims.

Throughout this application, various patents and non-patent publications are referenced. The disclosures of these patents and publications in their entireties are hereby incorporated by reference into this application in order to more fully describe the state of the art to which this invention pertains.

### REFERENCES

American Academy of Pediatrics. Committee on Infectious Diseases. 1998. Severe invasive group A streptococcal infections: a subject review. Pediatrics 101:136-40.
Ackerman, K. G., H. Huang, H. Grasemann, C. Puma, J. B. Singer, A. E. Hill, E. Lander, J. H. Nadeau, G. A. Churchill, J. M. Drazen, and D. R. Beier. 2005. Interacting genetic loci cause airway hyperresponsiveness. Physiol Genomics 21:105-11.
Adler, A., G. Cieslewicz, and C. G. Irvin. 2004. Unrestrained plethysmography is an unreliable measure of airway responsiveness in BALB/c and C57BL/6 mice. J Appl Physiol 97:286-92.
Barile, M. F. 1984. Immunization against Mycoplasma pneumoniae disease: a review. Isr J Med Sci 20:912-5.
Cimolai, N., G. P. Taylor, D. Mah, and B. J. Morrison. 1992. Definition and application of a histopathological scoring scheme for an animal model of acute Mycoplasma pneumoniae pulmonary infection. Microbiol. Immunol 36:465-78.
Fonseca Aten M, Rios A M, Mejias A, Chavez-Bueno S, Katz K, Gomez A M, McCracken G H, Ramilo O, Hardy R D. The Effect of Telithromycin in a Murine Model of Chronic Pulmonary Mycoplasmosis (abstr G-015). 104th ASM General Meeting, New Orleans, 2004.
Gonzalo, J. A., C. M. Lloyd, D. Wen, J. P. Albar, T. N. Wells, A. Proudfoot, A. C. Martinez, M. Dorf, T. Bjerke, A. J. Coyle, and J. C. Gutierrez-Ramos. 1998. The coordinated action of CC chemokines in the lung orchestrates allergic inflammation and airway hyperresponsiveness. J Exp Med 188:157-67.
Hamelmann, E., J. Schwarze, K. Takeda, A. Oshiba, G. L. Larsen, C. G. Irvin, and E. W. Gelfand. 1997. Noninvasive measurement of airway responsiveness in allergic mice using barometric plethysmography. Am J Respir Crit. Care Med 156:766-75.
Hardy, R. D., Jafri, H. S., Olsen, K., Hatfield, J., Iglehart, J., Rogers, B. B., Patel, P., Cassell, G., McCracken, G. H. and Ramilo, 0.2002. Mycoplasma pneumoniae induces chronic respiratory infection, airway hyperreactivity, and pulmonary inflammation: a murine model of infection-associated chronic reactive airway disease. Infect Immun. 70: 649-654.
Hardy, R. D., Jafri, H. S., Olsen, K., Wordemann, M., Hatfield, J., Rogers, B. B., Patel, P., Duffy, L., Cassell, G., McCracken, G. H. and Ramilo, 0.2001. Elevated cytokine and chemokine levels and prolonged pulmonary airflow resistance in a murine Mycoplasma pneumoniae pneumonia model: a microbiologic, histologic, immunologic, and respiratory plethysmographic profile. Infect Immun. 69: 3869-3876.
Hardy, R. D., Rios, A. M., Chavez-Bueno, S., Jafri, H. S., Hatfield, J., Rogers, B. B., McCracken, G. H. and Ramilo, 0.2003. Antimicrobial and immunologic activities of clarithromycin in a murine model of Mycoplasma pneumoniae-induced pneumonia. Antimicrob Agents Chemother. 47: 1614-1620.
Kraft, M., Cassell, G. H., Pak, J. and Martin, R. J. 2002. Mycoplasma pneumoniae and Chlamydiapneumoniae in asthma: effect of clarithromycin. Chest. 121: 1782-1788.
Krane, C. M., C. N. Fortner, A. R. Hand, D. W. McGraw, J. N. Lorenz, S. E. Wert, J. E. Towne, R. J. Paul, J. A. Whitsett, and A. G. Menon. 2001. Aquaporin 5-deficient mouse lungs are hyperresponsive to cholinergic stimulation. Proc Natl Acad Sci U S A 98:14114-9.
Mabe, S., J. Eller, and W. S. Champney. 2004. Structure-activity relationships for three macrolide antibiotics in Haemophilus influenzae. Curr Microbiol 49:248-54.
Rios, A. M., M. Fonseca-Aten, A. Mejias, S. Chavez-Bueno, K. Katz, A. M. Gomez, G. H. McCracken, Jr., O. Ramilo, and R. D. Hardy. 2005. Microbiologic and immunologic evaluation of a single high dose of azithromycin for treatment of experimental Mycoplasma pneumoniae pneumonia. Antimicrob Agents Chemother 49:3970-3.
Rios, A. M., A. Mejias, S. Chavez-Bueno, M. Fonseca-Aten, K. Katz, J. Hatfield, A. M. Gomez, H. S. Jafri, G. H. McCracken, Jr., O. Ramilo, and R. D. Hardy. 2004. Impact of cethromycin (ABT-773) therapy on microbiological, histologic, immunologic, and respiratory indices in a murine model of Mycoplasma pneumoniae lower respiratory infection. Antimicrob Agents Chemother 48:2897-904.
Rubin, B. K. 2004. Immunomodulatory properties of macrolides: overview and historical perspective. Am J Med 117 Suppl 9A:2S-4S.
Schwarze, J., E. Hamelmann, K. L. Bradley, K. Takeda, and E. W. Gelfand. 1997. Respiratory syncytial virus infection results in airway hyperresponsiveness and enhanced airway sensitization to allergen. J Clin Invest 100:226-33.
Tateda, K., Y. Ishii, T. Matsumoto, N. Furuya, M. Nagashima, T. Matsunaga, A. Ohno, S. Miyazaki, and K. Yamaguchi. 1996. Direct evidence for antipseudomonal activity of macrolides: exposure-dependent bactericidal activity and inhibition of protein synthesis by erythromycin, clarithromycin, and azithromycin. Antimicrob Agents Chemother 40:2271-5.
Tateda, K., Y. Ishii, T. Matsumoto, T. Kobayashi, S. Miyazaki, and K. Yamaguchi. 2000. Potential of macrolide antibiotics to inhibit protein synthesis of Pseudomonas aeruginosa: suppression of virulence factors and stress response. J Infect Chemother 6:1-7.
van Schaik, S. M., G. Enhorning, I. Vargas, and R. C. Welliver. 1998. Respiratory syncytial virus affects pulmonary function in BALB/c mice. J Infect Dis 177:269-76.
Wozniak, D. J., and R. Keyser. 2004. Effects of subinhibitory concentrations of macrolide antibiotics on Pseudomonas aeruginosa. Chest 125:62 S-69S; quiz 69S.
Fonseca-Aten, M., A. M. Rios, A. Mejias, S. Chavez-Bueno, K. Katz, A. M. Gomez, G. H. McCracken, Jr., and R. D. Hardy. 2005. Mycoplasma pneumoniae induces host-dependent pulmonary inflammation and airway obstruction in mice. Am J Respir Cell Mol Biol 32:201-10.
Himmelreich, R., H. Hilbert, H. Plagens, E. Pirkl, B. C. L1, and R. Herrmann. 1996.

Complete sequence analysis of the genome of the bacterium Mycoplasma pneumoniae. Nucleic Acids Res 24:4420-49.
52. Somerville, J. E., Jr., Cassiano, L., Bainbridge, B., Cunningham, M. D. & Darveau, R. P. A novel Escherichia coli lipid A mutant that produces an antiinflammatory lipopolysaccharide. J Clin Invest 97, 359-65 (1996).
53. Low, K. B. et al. Lipid A mutant Salmonella with suppressed virulence and TNFalpha induction retain tumor-targeting in vivo. Nat Biotechnol 17, 37-41 (1999).
54. Cognet, I. et al. Expression of recombinant proteins in a lipid A mutant of Escherichia coli BL21 with a strongly reduced capacity to induce dendritic cell activation and maturation. J Immunol Methods 272, 199-210 (2003).
55. Domenighini, M., Magagnoli, C., Pizza, M. & Rappuoli, R. Common features of the NAD-binding and catalytic site of ADP-ribosylating toxins. Mol Microbiol 14, 41-50 (1994).
56. Domenighini, M., Montecucco, C., Ripka, W. C. & Rappuoli, R. Computer modeling of the NAD binding site of ADP-ribosylating toxins: active-site structure and mechanism of NAD binding. Mol Microbiol 5, 23-31 (1991).
57. Domenighini, M. & Rappuoli, R. Three conserved consensus sequences identify the NAD-binding site of ADP-ribosylating enzymes, expressed by eukaryotes, bacteria and T-even bacteriophages. Mol Microbiol 21, 667-74 (1996).
58. Althaus, F. R. & Richter, C. ADP-ribosylation of proteins. Enzymology and biological significance. Mol Biol Biochem Biophys 37, 1-237 (1987).
59. Hewlett, E. L., Sauer, K. T., Myers, G. A., Cowell, J. L. & Guerrant, R. L.

Induction of a novel morphological response in Chinese hamster ovary cells by pertussis toxin. Infect Immun 40, 1198-203 (1983).
60. Xu, Y. & Barbieri, J. T. Pertussis toxin-mediated ADP-ribosylation of target proteins in Chinese hamster ovary cells involves a vesicle trafficking mechanism. Infect Immun 63, 825-32 (1995).
61. Mekalanos, J. J., Collier, R. J. & Romig, W. R. Enzymic activity of cholera toxin. II. Relationships to proteolytic processing, disulfide bond reduction, and subunit composition. J Biol Chem 254, 5855-61 (1979).
62. Moss, J., Stanley, S. J., Morin, J. E. & Dixon, J. E. Activation of choleragen by thiol: protein disulfide oxidoreductase. J Biol Chem 255, 11085-7 (1980). 63.
64. Moss, J. et al. Activation by thiol of the latent NAD glycohydrolase and ADP-ribosyltransferase activities of Bordetella pertussis toxin (islet-activating protein). J Biol Chem 258, 11879-82 (1983).
69. Reddy, S. P., Rasmussen, W. G. & Baseman, J. B. Isolation and characterization of transposon Tn4001-generated, cytadherence-deficient transformants of Mycoplasa pneumoniae and Mycoplasma genitalium. FEMS Immunol Med Microbiol 15, 199-211 (1996).
70. Pour-El, I., Adams, C. & Minion, F. C. Construction of mini-Tn4001 tet and its use in Mycoplasma gallisepticum. Plasmid 47, 129-37 (2002).
Dube, P. H., S. A. Handley, P. A. Revell, and V. L. Miller. 2003. The rovA mutant of Yersinia enterocolitica displays differential degrees of virulence depending on the route of infection. Infect Immun 71:3512-20.
17. Dube, P. H., P. A. Revell, D. D. Chaplin, R. G. Lorenz, and V. L. Miller. 2001.

A role for IL-1 alpha in inducing pathologic inflammation during bacterial infection. Proc Natl Acad Sci USA 98:10880-5.
Kraft, M., G. H. Cassell, J. E. Henson, H. Watson, J. Williamson, B. P. Marmion, C. A. Gaydos, and R. J. Martin. 1998. Detection of Mycoplasma pneumoniae in the airways of adults with chronic asthma. Am J Respir Crit. Care Med 158:998-1001.

TABLE 3

| | | | | [STS - related or absent] | | |
|---|---|---|---|---|---|---|
| Toxin | AA | [R] (SEQ ID NO:) | AA | (SEQ ID NO:) | AA | [E] (SEQ ID NO:) |
| MYPE | 24 | KFLYRVDYK (80) | 66 | EDCFINAFETVN (81) | 150 | KFTNKFEWFTT (82) |
| MPN | 6 | VREVYRVDLR (83) | 43 | RSYFISTSETPT (84) | 135 | SFAYQREWFTD (85) |
| PT | 4 | ATVYRDDLR (86) | 36 | AFVSTSSSRR (87) | 125 | AAYQREWFAD (88) |
| SpyA | 96 | IVWYRYWYE (89) | 142 | KHSFMSTTALKN (90) | 181 | AVPSEVELLFP (91) |
| EDIN | 81 | VYWYRLLNL (92) | 13 | REDGYSSTQLVS (93) | 165 | AYYGQCEVLLP (94) |
| CT | 3 | DKLYRADSR (95) | 56 | DDGYWSTSISLR (96) | 106 | DHDDEQEVSAL (97) |
| C3bot | 91 | IMLFRCDDP (98) | 136 | EYGYISTSLMNV (99) | 175 | AFAGQLENLLP (100) |
| ToxA_SALTY | 1 | VDFVYRVDS (101) | 37 | YIATTSVNQT (102) | 51 | RLQREYVSTL (103) |
| Tox2_SALTY | 1 | VDFVYRVDS (105) | 37 | YIATISDINEA (106) | 51 | RLQSEYVSVN (106) |
| ART1_BURCE | 380 | EGALYRPSA (107) | 41 | GYLGTYRSAF (108) | 32 | PAQGEVAAM (109) |
| ART4_BURCE | 438 | PFALFRPSQ (110) | 41 | GYVGTFTGRA (111) | 36 | PRDGEVAAM (112) |

1. A chimeric protein comprising a CARDS toxin or biologically active fragment thereof and a ligand for targeting the CARDS toxin or biologically active fragment thereof to a cell that is to be killed by the CARDS toxin or biologically active fragment thereof.

2. The chimeric protein of claim 1, wherein the ligand is an antibody or ligand that specifically reacts with a cancer antigen.

3. The chimeric protein of claim 1, wherein the ligand is an antibody or ligand that specifically reacts with a receptor on a muscle cell.

4. A method for killing a tumor cell in a subject, comprising contacting the tumor cell in the subject with the chimeric protein of claim 1.

5. A method for treating a muscle spasm in a subject, comprising contacting a muscle of the muscle spasm with the chimeric protein of claim 1.

6. An isolated polypeptide comprising the amino acid sequence of SEQ ID NO:79 (M. penetrans toxin) or a biologically active fragment thereof.

7. An isolated nucleic acid comprising the nucleotide sequence of SEQ ID NO:77 (M. penetrans WT sequence).

8. An isolated nucleic acid comprising the nucleotide sequence of SEQ ID NO:78 (UGG modified coding sequence of M. penetrans toxin).

9. (canceled)

10. An antibody that specifically binds a polypeptide comprising the amino acid sequence of SEQ ID NO:79 or an antigenic fragment thereof.

11. A method of diagnosing infection by Mycoplasma penetrans in a subject, comprising contacting a biological sample from the subject with a the polypeptide of claim 6 or an antigenic fragment thereof under conditions whereby an antigen/antibody complex can form and detecting formation of an antigen/antibody complex, thereby diagnosing infection by Mycoplasma penetrans in the subject.

12. A method of diagnosing infection by Mycoplasma penetrans in a subject comprising contacting a biological sample from the subject with an antibody of claim 10 under conditions whereby an antigen/antibody complex can form and detecting formation of an antigen/antibody complex, thereby diagnosing infection by Mycoplasma penetrans in the subject.

13. A method of diagnosing infection by Mycoplasma penetrans in a subject, comprising contacting a biological sample from the subject with the nucleic acid of claim 7 and/or a nucleic acid comprising the nucleotide sequence of SEQ ID NO:78 (M. penetrans coding sequence with UGG modifications) under conditions whereby hybridization of nucleic acid molecules can occur to form a hybridization complex and detecting the hybridization complex, thereby diagnosing infection by Mycoplasma penetrans in the subject.

14. A kit for diagnosing an infection by Mycoplasma penetrans in a subject, comprising the polypeptide of claim 6 or an antigenic fragment thereof, an antibody that specifically binds a polypeptide comprising the amino acid sequence of SEQ ID NO:79, a nucleic acid comprising the nucleotide sequence of SEQ ID NO:77 (WT), a nucleic acid comprising the nucleotide sequence of SEQ ID NO:78 (UGG modified) and any combination thereof.

15. A method of detecting an M. penetrans toxin or an antigenic fragment thereof in a sample, comprising:
a) contacting the sample with the antibody of claim 10 under conditions whereby an antigen/antibody complex can form; and
b) detecting formation of the antigen/antibody complex, thereby detecting M. penetrans toxin or an antigenic fragment in the sample.

16. A method of detecting an antibody to M. penetrans toxin in a sample, comprising:
a) contacting the sample with the polypeptide of claim 6 or an antigenic fragment thereof under conditions whereby an antigen/antibody complex can form; and
b) detecting formation of the antigen/antibody complex, thereby detecting an antibody to M. penetrans toxin in the sample.

17. A method of detecting a nucleic acid comprising a nucleotide sequence encoding M. penetrans toxin in a sample, comprising:
a) contacting the sample with an oligonucleotide comprising a nucleotide sequence that is complementary to the nucleotide sequence of claim 7, under conditions whereby nucleic acid hybridization can occur; and
b) detecting nucleic acid hybridization, thereby detecting nucleic acid comprising the nucleotide sequence encoding M. penetrans toxin in the sample.

18. A method of eliciting an immune response in a subject, comprising administering to the subject an effective amount of the polypeptide of claim 6 or an immunogenic fragment thereof.

19. A method of eliciting an immune response in a subject, comprising administering to the subject an effective amount of the nucleic acid of claim 7 and/or a nucleic acid comprising the nucleotide sequence of SEQ ID NO:78 (UGG modified).

20. A method of providing passive immunity to a subject, comprising administering to the subject an effective amount of the antibody of claim 10.

21. A method of treating or preventing infection by Mycoplasma penetrans in a subject, comprising administering to the subject an effective amount of the polypeptide of claim 6 or an immunogenic fragment thereof.

22. A method of treating or preventing infection by Mycoplasma penetrans in a subject, comprising administering to the subject an effective amount of the nucleic acid of claim 7 and/or a nucleic acid comprising the nucleotide sequence of SEQ ID NO:78 (UGG modified).

23. A method of treating or preventing infection by Mycoplasma penetrans in a subject, comprising administering to the subject an effective amount of the antibody of claim 10.

24. A method of identifying a substance having the ability to inhibit the binding activity of M. penetrans toxin, comprising contacting the substance with the polypeptide of claim 6 or a biologically active fragment thereof under conditions whereby binding can occur and detecting a decrease in the amount of binding in the presence of the substance as compared to a control amount of binding in the absence of the substance, thereby identifying a substance having the ability to inhibit the binding activity of the M. penetrans toxin.

25. A method of identifying a substance having the ability to enhance the immunogenic activity of M. penetrans toxin, comprising contacting the substance with the polypeptide of claim 6 or an immunogenic fragment thereof under conditions whereby a measurable immune response can be elicited and detecting in increase in the amount of immune response in the presence of the substance, as compared to a control amount of immune response in the absence of the substance, thereby identifying a substance having the ability to enhance immunogenic activity of the M. penetrans toxin.

26. A method of identifying a substance having the ability to inhibit the ADP-ribosylating activity of M. penetrans toxin, comprising contacting the substance with the polypeptide of claim 6 or biologically active fragment thereof under conditions whereby ADP ribosylation can occur and detecting a decrease in the amount of ADP ribosylation in the presence of the substance as compared to a control amount of ADP ribosylation in the absence of the substance, thereby identifying a substance having the ability to inhibit the ADP ribosylating activity of the M. penetrans toxin.

27. A method of identifying a substance having the ability to inhibit the cytopathology-inducing activity of M. penetrans toxin, comprising contacting the substance with the polypeptide of claim 6 or a biologically active fragment thereof under conditions whereby cytopathology of target cells can be induced and detecting a decrease in the amount of cytopathology in the presence of the substance, as compared to a control amount of cytopathology in the absence of the substance, thereby identifying a substance having the ability to inhibit the cytopathology-inducing activity of the M. penetrans toxin or biologically active fragment thereof.

28. A method of treating or preventing a detrimental immune response in a subject, comprising administering to the subject an effective amount of CARDS toxin or an active fragment thereof and/or an effective amount of a nucleic acid encoding CARDS toxin or an active fragment thereof.

29. A method of enhancing an immunomodulating effect of a substance, comprising combining the substance with CARDS toxin or an active fragment thereof and/or with a nucleic acid encoding CARDS toxin or an active fragment thereof.

30-31. (canceled)

32. A method of treating or preventing an autoimmune disorder in a subject, comprising administering to the subject an effective amount of CARDS toxin or an active fragment thereof and/or a nucleic acid encoding a CARDS toxin or active fragment thereof.

33. A method of reducing the likelihood of transplant rejection in a transplant recipient, comprising administering to the subject an effective amount of CARDS toxin or an active fragment thereof and/or a nucleic acid encoding a CARDS toxin or active fragment thereof.

34. The method of 33, wherein the CARDS toxin or active fragment thereof is administered to the transplant recipient, before, concurrently and/or after transplantation.

35. A method of modulating an immune response in a subject, comprising administering to the subject an effective amount of CARDS toxin or an active fragment thereof and/or a nucleic acid encoding CARDS toxin or an active fragment thereof.

36. A method treating or preventing an obstructive airway disorder in a subject, comprising administering to the subject an effective amount of an inhibitor of CARDS toxin activity.

37. The method of claim 36, wherein the obstructive airway disease is asthma, allergy, pneumonia, tracheobronchitis, pharyngitis, croup and/or bronchiolitis in any combination.

38. The method of claim 36, wherein the obstructive airway disorder is asthma.

39. The method of claim 35, wherein the inhibitor of CARDS toxin activity is an antibody that specifically binds CARDS toxin.
